(19) United States
(12) Patent Application Publication
(10) Pub. No.: US 2010/0015070 A1
(43) Pub. Date: Jan. 21, 2010

(54) USE OF NATURAL, RECOMBINANT AND SYNTHETIC RESILINS IN COSMETICS

(75) Inventors: Claus Bollschweiler, Heidelberg (DE); Burghard Liebmann, Bensheim (DE); Marcus Fehr, Speyer (DE); Daniel Hümmerich, Mannheim (DE); Arne Ptock, Nuess (DE); Evelyn Raunft, Bohl-Iggelheim (DE)

Correspondence Address: CONNOLLY BOVE LODGE & HUTZ, LLP, P O BOX 2207, WILMINGTON, DE, 19899, US

(73) Assignee: BASF SE, Ludwigshafen (DE)

(21) Appl. No.: 12/513,709

(22) PCT Filed: Nov. 7, 2007
(86) PCT No.: PCT/EP2007/062005

§ 371 Date: May 6, 2009

(30) Foreign Application Priority Data
Nov. 8, 2006 (EP) 06123659.2

(51) Int. Cl. A61Q 17/04; C07K 5/00; C07K 7/06; C07K 7/08; C07K 14/00; C12P 21/02

(52) U.S. Cl. 424/59; 530/330; 530/329; 530/328; 530/327; 530/326; 530/325; 530/324; 530/350; 435/69.1

(57) ABSTRACT

Use of resilin proteins in cosmetics, wherein the resilin proteins comprise between one and 100 repeat units with the consensus sequence SXXYGXP, where S is a serine, X is any desired amino acid, Y is a tyrosine, G is a glycine and P is a proline.

The present invention relates to the use of natural, recombinant and synthetic resilin proteins in cosmetics, in particular in skin cosmetics.

### PRIOR ART

WO 2004/104042 describes the production of a natural proresilin fragment as well as the further processing through crosslinking to give a bioelastomer and its use.

WO 2004/104043 describes protein sequences based on repetitive sequences of natural resilin proteins, as well as the further processing through crosslinking to give bioelastomers and their use.

### DESCRIPTION OF THE INVENTION

The present invention relates to novel cosmetic or dermatological compositions which comprise, as active ingredient, at least one natural, recombinant or synthetic resilin from insects or an analogue of such a protein.

The present invention relates in particular to compositions which are present as products for hair treatment, skincare products, make-up products or sun protection products based on a resilin protein from insects.

The use of proteins is well known; in cosmetics, they are often used, in particular silk proteins of the caterpillar Bombyx mori, this use having been described in detail in Kaplan et al.: Silks; Biomaterials. Novel Materials from Biological Sources 1-53 Stockton press; New York (1991). However, the silk proteins do not have good elastic properties when they are present as threads or films, and are therefore unable to satisfactorily serve as film formers.

The properties of resilin are described in Gosline et al.; Philosophical Transactions of the Royal Society of London—Series B: Biological Sciences. 357(1418):121-32, (2002). They are known for their good elastic properties and the high fraction of elastically stored energy (resilience).

It has now been found that resilin proteins, in addition to their known properties, impart unexpected properties to cosmetic or dermatological compositions and in particular are able to improve the hydrating or softening effect. Moreover, the resilins behave as good film formers and have in particular a low surface density.

The present invention therefore provides, as a novel technical product, a cosmetic or dermatological composition which comprises an effective amount of at least one natural recombinant or synthetic resilin or an analogue of such a protein.

According to one embodiment of the cosmetic compositions according to the invention, the resilin is preferably natural resilin obtained from insects.

According to a specific embodiment of the cosmetic compositions according to the invention, the resilins are preferably recombinant resilins from insects. For this purpose, a natural DNA sequence coding for resilin or resilin fragments is isolated from insects, heterologously expressed in an expression organism and the recombinant resilin protein is isolated therefrom. The nucleotide sequence of the isolated DNA can additionally be altered using biomolecular methods.

According to a preferred embodiment of the cosmetic compositions according to the invention, the resilins are preferably synthetic resilins. Here, gene sequences which code for protein sequences which are based on repeat units of natural resilin proteins are synthesized using chemical and biomolecular methods. The synthetic genes are heterologously expressed in an expression organism and the synthetic resilin protein is isolated therefrom.

The recombinant and synthetic resilins have the advantage that they have a peptide sequence whose physicochemical properties can be adapted depending on the desired cosmetic or dermatological applications.

According to the invention, the compositions can comprise at least one natural or recombinant or synthetic resilin protein in an effective amount.

Of suitability for producing cosmetic or dermatological compositions are resilin proteins which comprise a large number of repeat units, preferably 1-100, particularly preferably 10-40, and advantageously 15-33 repeat units with the consensus sequence SXXYGXP, where S is a serine, X is any desired amino acid, Y is a tyrosine, G is a glycine and P is a proline. Preferably, X is selected from the group of charged and polar amino acids.

In a preferred embodiment of the invention, the repeat unit comprises the consensus sequence;

X₁X₂X₃X₄SX₅X₆YGX₇PX₈X₉X₁₀X₁₁

where
X₁ is any desired amino acid or not an amino acid
X₂ is any desired amino acid or not an amino acid
X₃ is any desired amino acid or not an amino acid
X₄ is a proline, alanine, threonine or serine
S is a serine
X₅ is a charged or polar amino acid
X₆ is a charged or polar amino acid
Y is a tyrosine
G is a glycine
X₇ is a proline, alanine, threonine or serine
P is a proline
X₈ is a glycine, alanine, threonine or serine
X₉ is a glycine, a polar amino acid or not an amino acid
X₁₀ is a glycine, a polar amino acid or not an amino acid

X₁₁ is any desired amino acid or not an amino acid.

In one embodiment, X₁, if present, is a G, Y, A or N, preferably a G.

In one embodiment, X₂, if present, is a basic amino acid, G, L, Q, preferably G.

In one embodiment, X₃, if present, is an R, K, T or P;

X₄ is preferably a P;
X₅ is preferably a D, T or S;
X₆ is preferably an S, Q or T;
X₇ is preferably an A;
X₈ is preferably a G;
X₉, if present, is preferably a G, Q or S, most preferably a G;
X₁₀, if present, is preferably a G, S or N, most preferably a G;
X₁₁, if present, is preferably a G, Q, P, S or N.

In one embodiment, at least one of the amino acids X₈, X₉ and X₁₀ is a G, preferably X₈.

The term consensus sequence—as used here—refers to an amino acid (or nucleotide) sequence which clearly stipulates amino acids often occurring at a certain position with regard to radical and position, whereas other amino acids are not specified more precisely but are replaced by the spacer X instead of the customarily used single-letter code for amino acids. The single-letter code for amino acids which is used here is known to the person skilled in the art.

The repeat units of the resilin proteins may be of the same type or different.

The repeat units can be joined by linkers which preferably comprise 1 to 30 amino acids, particularly preferably 1 to 20 amino acids. The amino acid sequence of a linker can be derived from other proteins, preferably structural proteins, or not have a natural pattern or be entirely absent.

The distance of an amino acid within one repeat unit to the equivalent amino acid within the adjacent repeat unit can be 8 to 50 amino acids, preferably 8 to 34, and particularly preferably 10 to 25 and most preferably 10 to 16 amino acids.

It is possible for any desired number of repeat units, preferably 1 to 100, particularly preferably 10-65 and most preferably 15 to 35, to be joined together.

In one embodiment, the protein comprises the repeat unit GAPGGGNGGRPSDTY or GAPGGGNGGRPSSSY.

In a further embodiment, the sequence of the protein or parts thereof correspond(s) to SEQ ID NO 1 or parts of this sequence. Amino acid 1-15 of SEQ ID NO: 1 constitutes a T7 tag which has been fused with the actual resilin protein. The resilin proteins according to the invention can be produced with or without such tags. It is also possible to replace the T7 tag by other similarly effective tags which can facilitate the identification and isolation of the resilin proteins.

In a further embodiment, the sequence of the protein or parts thereof correspond(s) to SEQ ID NO 3 or parts of this sequence.

In a further embodiment, the sequence of the protein or parts thereof correspond(s) to the peptide sequence or parts thereof of the resilin protein from other insects, such as, for example, Anopheles gambiae, Apis mellifera, Ctenocephalides felis, Haematobia irritans exigua. The peptide sequences of these proteins are published in WO04/104042, to which reference is hereby expressly made.

In a further embodiment, the resilin protein is part of a hybrid protein. The hybrid protein consists here of one or more resilin sequences or sequences derived therefrom, or fragments of these sequences, as well as one or more further protein sequences or fragments of these sequences. These further proteins can preferably be structural proteins, such as, for example, silk protein, spider silk protein, keratin, elastin, collagen, gluten, abductin, mussel byssus protein.

Recombinant and synthetic resilin proteins are produced by expression of natural or synthetic gene sequences. Methods for the isolation and modification of natural gene sequences are known to the person skilled in the art.

One possibility for producing synthetic gene sequences is described in Huemmerich et al.; Biochemistry. 43(42):13604-12, (2004).

The expression systems for proteins are well known and have been described in Sambrook et al.: Molecular cloning: A Laboratory Manual; 3rd Ed. Cold Spring Harbour Laboratory Press; Cold Spring Harbour (2001).

Nonlimiting examples of prokaryotic expression organisms are Escherichia coli, Bacillus subtilis, Bacillus megaterium, Corynebacterium glutamicum etc. Nonlimiting examples of eukaryotic expression organisms are yeasts, such as Saccharomyces cerevisiae, Pichia pastoris etc., filamentous fungi, such as Aspergillus niger, Aspergillus oryzae, Aspergillus nidulans, Trichoderma reesei, Acremonium chrysogenum etc., mammal cells, such as Hela cells, COS cells, CHO cells etc., insect cells, such as Sf9 cells, MEL cells etc., plants or plant cells such as Solanum tuberosum, Nicotiana etc.

In the compositions according to the invention, the expression “effective amount” of at least one natural, recombinant or synthetic resilin protein of insects or an analogous compound corresponds to a quantitative fraction of from about 0.001 to 30% by weight, but preferably from 0.01 to 15% by weight, based on the total weight of the composition, where the quantitative fraction can vary depending on the type of cosmetic or dermatological composition.

Besides the polypeptide sequences shown above, functional equivalents, functional derivatives and salts of this sequence in particular are also preferred.

According to the invention, “functional equivalents” are understood in particular as meaning also mutants which have an amino acid other than that specifically given in at least one sequence position of the abovementioned amino acid sequences but nevertheless have one of the abovementioned properties. “Functional equivalents” thus comprise the mutants obtainable by one or more amino acid additions, substitutions, deletions and/or inversions, where the specified changes can arise in any sequence position provided they lead to a mutant with the profile of properties according to the invention. Functional equivalence is in particular also present if the reactivity patterns between mutant and unchanged polypeptide are in qualitative agreement. “Functional equivalents” in the above sense are also “precursors” of the described polypeptides, and “functional derivatives” and “salts” of the polypeptides.

Here, “precursors” are natural or synthetic precursors of the polypeptides with or without the desired biological activity.

Examples of suitable amino acid substitutions are given in the table below:

| Original radical | Examples of substitution |
|---|---|
| Ala | Ser |
| Arg | Lys |
| Asn | Gln; His |
| Asp | Glu |
| Cys | Ser |
| Gln | Asn |
| Glu | Asp |
| Gly | Pro |
| His | Asn; Gln |
| Ile | Leu; Val |
| Leu | Ile; Val |
| Lys | Arg; Gln; Glu |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

The expression “salts” is understood as meaning either salts of carboxyl groups or acid addition salts of amino groups of the protein molecules according to the invention. Salts of carboxyl groups can be produced in a manner known per se and comprise inorganic salts, such as, for example, sodium, calcium, ammonium, iron and zinc salts, and also salts with organic bases, such as, for example amines, such as triethanolamine, arginine, lysine, piperidine and the like. Acid addition salts, such as, for example, salts with mineral acids, such as hydrochloric acid or sulfuric acid and salts with organic acids, such as acetic acid and oxalic acid, are likewise provided by the invention.

“Functional derivatives” of polypeptides according to the invention can likewise be produced on functional amino acid side groups or on their N- or C-terminal end using known techniques. Such derivatives comprise, for example, allphatic esters of carboxylic acid groups, amides of carboxylic acid groups, obtainable by reaction with ammonia or with a primary or secondary amine; N-acyl derivatives of free amino groups, prepared by reaction with acyl groups; or O-acyl derivatives of free hydroxy groups, prepared by reaction with acyl groups.

The invention further provides molecules which consist of couplings of a resilin protein and an effector molecule. All of the proteins already specified above are suitable as protein.

Effector molecules are understood below as meaning molecules which have a certain predictable effect. These may either be protein-like molecules, such as enzymes, or non-proteinogenic molecules, such as dyes, photoprotective agents, vitamins, provitamins, antioxidants and fatty acids, conditioners or compounds comprising metal ions.

Among the protein-like effector molecules, enzymes and proteins having specific binding properties are preferred. Among the enzymes, the following are preferred as effector molecules: oxidases, peroxidases, proteases, glucanases, mutanase, tyrosinases, laccases, metal-binding enzymes, lactoperoxidase, lysozyme, amyloglycosidase, glucose oxidase, super oxide dismutase, photolyase, T4 endonuclease, katalase, thioredoxin, thioredoxin reductase.

For the protein-like, but not enzymatic effector molecules, the following are preferred as effector molecules: hydrophobins, collagen, proteins binding carotenoid, proteins binding heavy metals, proteins binding odorants, proteins binding cellulose, proteins binding starch, proteins binding keratin.

Highly suitable protein-like effector molecules are also hydrolyzates of proteins of vegetable and animal sources, for example hydrolyzates of proteins of marine origin.

Among the non-protein-like effector molecules, carotenoids are preferred. According to the invention, carotenoids are understood as meaning the following compounds and their esterified or glycosylated derivatives: β-carotene, lycopene, lutein, astaxanthin, zeaxanthin, cryptoxanthin, citranaxanthin, canthaxanthin, bixin, β-Apo-4-carotenal, β-Apo-8-carotenal, β-Apo-8-carotenoic esters, neurosporene, echinenone, adonirubin, violaxanthin, torulene, torularhodin, individually or as a mixture. Preferably used carotenoids are β-carotene, lycopene, lutein, astaxanthin, zeaxanthin, citranaxanthin and canthaxanthin.

Further preferred effector molecules are UV photoprotective filters. These are understood as meaning organic substances which are able to absorb ultraviolet rays and release the absorbed energy again in the form of long-wave radiation, e.g. heat. The organic substances may be oil-soluble or water-soluble.

Oil-soluble UV-B filters which may be used are, for example, the following substances:
3-benzylidenecamphor and derivatives thereof, e.g. 3-(4-methylbenzylidene)camphor;
4-aminobenzoic acid derivatives, preferably 2-ethylhexyl 4-(dimethylamino)benzoate, 2-octyl 4-(dimethylamino)benzoate and amyl 4-(dimethylamino)benzoate;
esters of cinnamic acid, preferably 2-ethylhexyl 4-methoxycinnamate, propyl 4-methoxycinnamate, isoamyl 4-methoxycinnamate, isopentyl 4-methoxycinnamate, 2-ethylhexyl 2-cyano-3-phenylcinnamate (octocrylene);
esters of salicylic acid, preferably 2-ethylhexyl salicylate, 4-isopropylbenzyl salicylate, homomethyl salicylate;
derivatives of benzophenone, preferably 2-hydroxy-4-methoxybenzophenone, 2-hydroxy-4-methoxy-4′-methylbenzophenone, 2,2′-dihydroxy-4-methoxy-benzophenone;
esters of benzalmalonic acid, preferably di-2-ethylhexyl 4-methoxybenzmalonate;
triazine derivatives, such as, for example, 2,4,6-trianilino-(p-carbo-2′-ethyl-1′-hexyloxy)-1,3,5-triazine (octyltriazone) and dioctylbutamidotriazone (Uvasorb® HEB);
propane-1,3-diones, such as, for example, 1-(4-tert-butylphenyl)-3-(4′-methoxyphenyl)-propane-1,3-dione.

Suitable water-soluble substances are:

2-phenylbenzimidazole-5-sulfonic acid and the alkali metal, alkaline earth metal, ammonium, alkylammonium, alkanolammonium and glucammonium salts thereof;
sulfonic acid derivatives of benzophenones, preferably 2-hydroxy-4-methoxybenzo-phenone-5-sulfonic acid and its salts;
sulfonic acid derivatives of 3-benzylidenecamphor, such as, for example, 4-(2-oxo-3-bornylidenemethyl)benzenesulfonic acid and 2-methyl-5-(2-oxo-3-bornylidene)-sulfonic acid and salts thereof.

Particular preference is given to the use of esters of cinnamic acid, preferably 2-ethylhexyl 4-methoxycinnamate, isopentyl 4-methoxycinnamate, 2-ethylhexyl 2-cyano-3-phenylcinnamate (octocrylene).

Furthermore, the use of derivatives of benzophenone, in particular 2-hydroxy-4-methoxybenzophenone, 2-hydroxy-4-methoxy-4′-methylbenzophenone, 2,2′-dihydroxy-4-methoxybenzophenone, and the use of propane-1,3-diones, such as, for example, 1-(4-tert-butylphenyl)-3-(4′-methoxyphenyl)propane-1,3-dione is preferred.

Suitable typical UV-A filters are:

derivatives of benzoylmethane, such as, for example, 1-(4′-tert-butylphenyl)-3-(4′-methoxyphenyl)propane-1,3-dione, 4-tert-butyl-4′-methoxydibenzoylmethane or 1-phenyl-3-(4′-isopropylphenyl)propane-1,3-dione;
aminohydroxy-substituted derivatives of benzophenones, such as, for example, N,N-diethylaminohydroxybenzoyl n-hexylbenzoate.

The UV-A and UV-B filters can of course also be used in mixtures.

Suitable UV filter substances are given in the table below.

| | | CAS No. |
|---|---|---|
| No. | Substance | (=acid) |
| 1 | 4-Aminobenzoic acid | 150-13-0 |
| 2 | 3-(4′-Trimethylammonium)benzylidenebornan-2-one methyl | 52793-97-2 |
| | sulfate | |
| 3 | 3,3,5-Trimethylcyclohexyl salicylate | 118-56-9 |
| | (homosalate) | |
| 4 | 2-Hydroxy-4-methoxybenzophenone | 131-57-7 |
| | (oxybenzone) | |
| 5 | 2-Phenylbenzimidazole-5-sulfonic acid and its potassium, | 27503-81-7 |
| | sodium and triethanolamine salts | |
| 6 | 3,3′-(1,4-Phenylenedimethine)bis(7,7-dimethyl- | 90457-82-2 |
| | 2-oxobicyclo[2.2.1]heptane-1-methanesulfonic acid) and its | |
| | salts | |
| 7 | Polyethoxyethyl 4-bis(polyethoxy)aminobenzoate | 113010-52-9 |
| 8 | 2-Ethylhexyl 4-dimethylaminobenzoate | 21245-02-3 |
| 9 | 2-Ethylhexyl salicylate | 118-60-5 |
| 10 | 2-Isoamyl 4-methoxycinnamate | 71617-10-2 |
| 11 | 2-Ethylhexyl 4-methoxycinnamate | 5466-77-3 |
| 12 | 2-Hydroxy-4-methoxybenzophenone-5-sulfonic acid | 4065-45-6 |
| | (sulisobenzone) and the sodium salt | |
| 13 | 3-(4′-Sulfobenzylidene)bornan-2-one and salts | 58030-58-6 |
| 14 | 3-Benzylidenebornan-2-one | 16087-24-8 |
| 15 | 1-(4′-Isopropylphenyl)-3-phenylpropane-1,3-dione | 63260-25-9 |
| 16 | 4-Isopropylbenzyl salicylate | 94134-93-7 |
| 17 | 3-Imidazol-4-ylacrylic acid and its ethyl ester | 104-98-3 |
| 18 | Ethyl 2-cyano-3,3-diphenylacrylate | 5232-99-5 |
| 19 | 2′-Ethylhexyl 2-cyano-3,3-diphenylacrylate | 6197-30-4 |
| 20 | Menthyl o-aminobenzoate or: | 134-09-8 |
| | 5-methyl-2-(1-methylethyl)-2-aminobenzoate | |
| 21 | Glyceryl p-aminobenzoate or: | 136-44-7 |
| | 1-glyceryl 4-aminobenzoate | |
| 22 | 2,2′-Dihydroxy-4-methoxybenzophenone (dioxybenzone) | 131-53-3 |
| 23 | 2-Hydroxy-4-methoxy-4-methyl-benzophenone | 1641-17-4 |
| | (mexenone) | |
| 24 | Triethanolamine salicylate | 2174-16-5 |
| 25 | Dimethoxyphenylglyoxalic acid or: | 4732-70-1 |
| | 3,4-dimethoxyphenylglyoxal acidic sodium | |
| 26 | 3-(4′-Sulfobenzylidene)bornan-2-one and its salts | 56039-58-8 |
| 27 | 4-tert-Butyl-4′-methoxydibenzoylmethane | 70356-09-1 |
| 28 | 2,2′,4,4′-Tetrahydroxybenzophenone | 131-55-5 |
| 29 | 2,2′-Methylenebis[6-(2H-benzotriazol-2-yl)-4-(1,1,3,3,- | 103597-45-1 |
| | tetramethylbutyl)phenol] | |
| 30 | 2,2′-(1,4-Phenylene)bis-1H-benzimidazole-4,6- | 180898-37-7 |
| | disulfonic acid, Na salt | |
| 31 | 2,4-bis[4-(2-Ethylhexyloxy)-2-hydroxy]phenyl- | 187393-00-6 |
| | 6-(4-methoxyphenyl)-(1,3,5)-triazine | |
| 32 | 3-(4-Methylbenzylidene)camphor | 36861-47-9 |
| 33 | Polyethoxyethyl 4-bis(polyethoxy)paraaminobenzoate | 113010-52-9 |
| 34 | 2,4-Dihydroxybenzophenone | 131-56-6 |
| 35 | 2,2′-Dihydroxy-4,4′-dimethoxybenzophenone-5,5′- | 3121-60-6 |
| | disodium sulfonate | |
| 36 | Benzoic acid, 2-[4-(diethylamino)-2-hydroxybenzoyl]-, hexyl ester | 302776-68-7 |
| 37 | 2-(2H-Benzotriazol-2-yl)-4-methyl-6-[2-methyl-3-[1,3,3,3- | 155633-54-8 |
| | tetramethyl-1-[(trimethylsilyl)oxy]disiloxanyl]propyl]phenol | |
| 38 | 1,1-[(2,2′-Dimethylpropoxy)carbonyl]-4,4-diphenyl-1,3-butadiene | 363602-15-7 |

Besides the two abovementioned groups of primary photoprotective substances, it is also possible to use secondary photoprotective agents of the antioxidant type which interrupt the photochemical reaction chain which is triggered when UV radiation penetrates into the skin. Typical examples thereof are superoxide dismutase, catalase, tocopherols (vitamin E), coenzyme Q10, ubiquinanes, quinones and ascorbic acid (vitamin C).

A further group are antiirritants which have an antiinflammatory effect on skin damaged by UV light. Such substances are, for example, bisabolol, phytol and phytantriol.

Effector molecules according to the invention are also inorganic pigments which stop UV rays. Preference is given to pigments based on metal oxides and/or other metal compounds which are insoluble or sparingly soluble in water and chosen from the group of oxides of zinc (ZnO), titanium (TiO₂), iron (e.g. Fe₂O₃), zirconiu (ZrO₂), silicon (SiO₂), manganese (e.g. MnO), aluminum (Al₂O₃), cerium (e.g. Ce₂O₃), mixed oxides of the corresponding metals and mixtures of such oxides.

The inorganic pigments can be present here in coated form, i.e. are treated superficially. This surface treatment can consist, for example, in providing the pigments with a thin hydrophobic layer by a method known per se, as described in DE-A-33 14 742.

Further preferred effector molecules are vitamins, in particular vitamin A and esters thereof.

For the purposes of the present invention, retinoids are understood as meaning vitamin A alcohol (retinol) and its derivatives, such as vitamin A aldehyde (retinal), vitamin A acid (retinoic acid) and vitamin A ester (e.g. retinyl acetate, retinyl propinate and retinyl palmitate). The term retinoic acid here comprises both all-trans retinoic acid and also 13-cis-retinoic acid. The terms retinol and retinal preferably comprise the all-trans compounds. A preferred retinoid used for the suspensions according to the invention is all-trans-retinol, referred to below as retinol.

Further preferred effector molecules are vitamins, provitamins and vitamin precursors from the groups A, C, E and F, in particular 3,4-didehydroretinol, β-carotene (provitamin of vitamin A), ascorbic acid (vitamin C), and the palmitic esters, glucosides or phosphates of ascorbic acid, tocopherols, in particular α-tocopherol, and its esters, e.g. the acetate, the nicotinate, the phosphate and the succinate; also vitamin F, which is understood as meaning essential fatty acids, particularly linoleic acid, linolenic acid and arachidonic acid.

The vitamins, provitamins or vitamin precursors of the vitamin B group or derivatives thereof and the derivatives of 2-furanone to be used with preference according to the invention include, inter alia:

Vitamin B₁, trivial name thiamine, chemical name 3-[(4′-amino-2′-methyl-5′-pyrimidinyl)-methyl]-5-(2-hydroxyethyl)-4-methylthiazolium chloride.

Vitamin B₂, trivial name riboflavin, chemical name 7,8-dimethyl-10-(1-D-ribityl)-benzo[g]pteridine-2,4(3H,10H)-dione. In free form, riboflavin occurs, for example, in whey, other riboflavin derivatives can be isolated from bacteria and yeasts. A stereoisomer of riboflavin which is likewise suitable according to the invention is lyxoflavin, which can be isolated from fish meal or liver and bears a D-arabityl radical instead of the D-ribityl radical.

Vitamin B₃. The compounds nicotinic acid and nicotinamide (niacinamide) often bear this name. According to the invention, preference is given to nicotinamide.

Vitamin Br₅ (pantothenic acid and panthenol). Preference is given to using panthenol. Derivatives of panthenol which can be used according to the invention are, in particular, the esters and ethers of panthenol, and cationically derivatized panthenols. In a further preferred embodiment of the invention, derivatives of 2-furanone can also be used in addition to pantothenic acid or panthenol. Particularly preferred derivatives are the also commercially available substances dihydro-3-hydroxy-4,4-dimethyl-2(3H)-furanone with the trivial name pantolactone (Merck), 4-hydroxymethyl-γ-butyrolactone (Merck), 3,3-dimethyl-2-hydroxy-γ-butyrolactone (Aldrich) and 2,5-dihydro-5-methoxy-2-furanone (Merck), with all stereoisomers being expressly included.

These effector molecule compounds advantageously impart moisturizing and skin-calming properties to the resilin proteins according to the invention.

Vitamin B₆, which is not understood here as meaning a uniform substance, but the derivatives of 5-hydroxymethyl-2-methylpyridin-3-ol known under the trivial names pyridoxin, pyridoxamine and pyridoxal.

Vitamin B₇ (biotin), also referred to as vitamin H or “skin vitamin”. Biotin is (3aS,4S,6aR)-2-oxohexahydrothienol[3,4-d]imidazole-4-valeric acid.

Panthenol, pantolactone, nicotinamide and biotin are very particularly preferred according to the invention.

According to the invention, suitable derivatives (salts, esters, sugars, nucleotides, nucleosides, peptides and lipids) of the specified compounds can be used as effector molecules. As lipophilic, oil-soluble antioxidants from this group, preference is given to tocopherol and derivatives thereof, gallic esters, flavonoids and carotenoids and also butylhydroxytoluene/anisole. Preferred water-soluble antioxidants are amino acids, e.g. tyrosine and cysteine and derivatives thereof, and tannins, in particular those of vegetable origin.

Furthermore, preference is given to so-called peroxide decomposers, i.e. compounds which are able to decompose peroxides, particularly preferably lipid peroxides. These are understood as meaning organic substances, such as, for example, pyridine-2-thiol-3-carboxylic acid, 2-methoxypyrimidinolcarboxylic acids, 2-methoxypyridinecarboxylic acids, 2-dimethylaminopyrimidinolcarboxylic acids, 2-dimethylaminopyridinecarboxylic acids.

Triterpenes, in particular triterpenoic acids, such as ursolic acid, rosmaric acid, betulinic acid, boswellic acid and bryonolic acid.

A further preferred effector molecule is lipoic acid and suitable derivatives (salts, esters, sugars, nucleotides, nucleosides, peptides and lipids).

Further preferred effector molecules are fatty acids, in particular saturated fatty acids which carry an alkyl branch, particularly preferably branched eicosanoic acids, such as 18-methyleicosanoic acid.

Further preferred effector molecules are dyes, for example food dyes, semipermanent dyes, reactive or oxidation dyes. In the case of the oxidation dyes, it is preferred to link a component as effector molecule with the resilin proteins and then to couple oxidatively with the second dye component at the site of action, i.e. following application to skin. In the case of oxidation dyes it is also preferred to carry out the coupling of the dye components before the linkage with the resilin proteins.

The reactive dyes can also preferably be linked as a component as effector molecule with the resilin proteins and then be applied to the skin. In addition, those dyes which are linked as effector molecule with the resilin proteins can be used in decorative cosmetics by application to skin.

Suitable dyes are all customary hair dyes for the molecules according to the invention. Suitable dyes are known to the person skilled in the art from cosmetics and books, for example Schrader, Grundlagen und Rezepturen der Kosmetika [Fundamentals and formulations of cosmetics], Hüthig Verlag, Heidelberg, 1989, ISBN 3-7785-1491-1.

Particularly advantageous dyes are those specified in the list below. The Colour Index Numbers (CIN) are given in the Rowe Colour Index, 3rd edition, Society of Dyers and Colourists, Bradford, England, 1971.

| Chemical or other name | CIN | Color |
|---|---|---|
| Pigment Green | 10006 | green |
| Acid Green 1 | 10020 | green |
| 2,4-Dinitrohydroxynaphthalene-7-sulfonic acid | 10316 | yellow |
| Pigment Yellow 1 | 11680 | yellow |
| Pigment Yellow 3 | 11710 | yellow |
| Pigment Orange 1 | 11725 | orange |
| 2,4-Dihydroxyazobenzene | 11920 | orange |
| Solvent Red 3 | 12010 | red |
| 1-(2′-Chloro-4′-nitro-1′-phenylazo)-2-hydroxynaphthalene | 12085 | red |
| Pigment Red 3 | 12120 | red |
| Ceres Red; Sudan Red; Fett Red G | 12150 | red |
| Pigment Red 112 | 12370 | red |
| Pigment Red 7 | 12420 | red |
| Pigment Brown 1 | 12480 | brown |
| 4-(2′-Methoxy-5′-sulfodiethylamido-1′-phenylazo)-3-hydroxy-5″-chloro-2″,4″- | 12490 | red |
| dimethoxy-2-naphthanilide | | |
| Disperse Yellow 16 | 12700 | yellow |
| 1-(4-Sulfo-1-phenylazo)-4-aminobenzene-5-sulfonic acid | 13015 | yellow |
| 2,4-Dihydroxyazobenzene-4′-sulfonic acid | 14270 | orange |
| 2-(2,4-Dimethylphenylazo-5-sulfonic acid)-1-hydroxy- | 14700 | red |
| naphthalene-4-sulfonic acid | | |
| 2-(4-Sulfo-1-naphthylazo)-1-naphthol-4-sulfonic acid | 14720 | red |
| 2-(6-Sulfo-2,4-xylylazo)-1-naphthol-5-sulfonic acid | 14815 | red |
| 1-(4′-Sulfophenylazo)-2-hydroxynaphthalene | 15510 | orange |
| 1-(2-Sulfo-4-chloro-5-carboxy-1-phenylazo)-2-hydroxy- | 15525 | red |
| naphthalene | | |
| 1-(3-Methylphenylazo-4-sulfo)-2-hydroxynaphthalene | 15580 | red |
| 1-(4′,(8′)-Sulfonaphthylazo)-2-hydroxynaphthalene | 15620 | red |
| 2-Hydroxy-1,2′-azonaphthalene-1′-sulfonic acid | 15630 | red |
| 3-Hydroxy-4-phenylazo-2-naphthylcarboxylic acid | 15800 | red |
| 1-(2-Sulfo-4-methyl-1-phenylazo)-2-naphthylcarboxylic acid | 15850 | red |
| 1-(2-Sulfo-4-methyl-5-chloro-1-phenylazo)-2-hydroxy-naphthalene- | 15865 | red |
| 3-carboxylic acid | | |
| 1-(2-Sulfo-1-naphthylazo)-2-hydroxynaphthalene-3-carboxylic | 15880 | red |
| acid | | |
| 1-(3-Sulfo-1-phenylazo)-2-naphthol-6-sulfonic acid | 15980 | orange |
| 1-(4-Sulfo-1-phenylazo)-2-naphthol-6-sulfonic acid | 15985 | yellow |
| Allura Red | 16035 | red |
| 1-(4-Sulfo-1-naphthylazo)-2-naphthol-3,6-disulfonic acid | 16185 | red |
| Acid Orange 10 | 16230 | orange |
| 1-(4-Sulfo-1-naphthylazo)-2-naphthol-6,8-disulfonic acid | 16255 | red |
| 1-(4-Sulfo-1-naphthylazo)-2-naphthol-3,6,8-trisulfonic acid | 16290 | red |
| 8-Amino-2-phenylazo-1-naphthol-3,6-disulfonic acid | 17200 | red |
| Acid Red 1 | 18050 | red |
| Acid Red 155 | 18130 | red |
| Acid Yellow 121 | 18690 | yellow |
| Acid Red 180 | 18736 | red |
| Acid Yellow 11 | 18820 | yellow |
| Acid Yellow 17 | 18965 | yellow |
| 4-(4-Sulfo-1-phenylazo)-1-(4-sulfophenyl)-5-hydroxypyrazolone- | 19140 | yellow |
| 3-carboxylic acid | | |
| Pigment Yellow 16 | 20040 | yellow |
| 2,6-(4′-Sulfo-2″,4″-dimethyl)bis(phenylazo)-1,3-dihydroxy- | 20170 | orange |
| benzene | | |
| Acid Black 1 | 20470 | black |
| Pigment Yellow 13 | 21100 | yellow |
| Pigment Yellow 83 | 21108 | yellow |
| Solvent Yellow | 21230 | yellow |
| Acid Red 163 | 24790 | red |
| Acid Red 73 | 27290 | red |
| 2-[4′-(4″-Sulfo-1″-phenylazo)-7′-sulfo-1′-naphthylazo]-1-hydroxy- | 27755 | black |
| 7-aminonaphthalene-3,6-di-sulfonic acid | | |
| 4′-[(4″-Sulfo-1″-phenylazo)-7′-sulfo-1′-naphthylazo]-1-hydroxy- | 28440 | black |
| 8-acetylaminonaphthalene-3,5-disulfonic acid | | |
| Direct Orange 34, 39, 44, 46, 60 | 40215 | orange |
| Food Yellow | 40800 | orange |
| trans-β-Apo-8′-Carotenaldehyde (C₃₀) | 40820 | orange |
| trans-Apo-8′-Carotenic acid(C₃₀)-ethyl ester | 40825 | orange |
| Canthaxanthin | 40850 | orange |
| Acid Blue 1 | 42045 | blue |
| 2,4-Disulfo-5-hydroxy-4′-4″-bis(dimethylamino)triphenylcarbinol | 42051 | blue |
| 4-[(-4-N-Ethyl-p-sulfobenzylamino)phenyl-(4-hydroxy- | 42053 | green |
| 2-sulfophenyl)(methylene)-1-(N-ethyl-N-p-sulfobenzyl)- | | |
| 2,5-cyclohexadienimine] | | |
| Acid Blue 7 | 42080 | blue |
| (N-Ethyl-p-sulfobenzylamino)phenyl-(2-sulfophenyl)methylene- | 42090 | blue |
| (N-ethyl-N-p-sulfobenzyl)^{□2,5}-cyclohexadienimine | | |
| Acid Green 9 | 42100 | green |
| Diethyldisulfobenzyldi-4-amino-2-chloro-di-2-methyl- | 42170 | green |
| fuchsonimmonium | | |
| Basic Violet 14 | 42510 | violet |
| Basic Violet 2 | 42520 | violet |
| 2′-Methyl-4′-(N-ethyl-N-m-sulfobenzyl)amino- | 42735 | blue |
| 4″-(N-diethyl)amino-2-methyl-N-ethyl-N-m-sulfobenzyl- | | |
| fuchsonimmonium | | |
| 4″-(N-Dimethyl)amino-4″-(N-phenyl)aminonaphtho-N-dimethyl- | 44045 | blue |
| fuchsonimmonium | | |
| 2-Hydroxy-3,6-disulfo-4,4′-bis-dimethylaminonaphtho- | 44090 | green |
| fuchsonimmonium | | |
| Acid Red 52 | 45100 | red |
| 3-(2′-Methylphenylamino)-6-(2′-methyl-4′-sulfophenylamino)- | 45190 | violet |
| 9-(2″-carboxyphenyl)xanthenium salt | | |
| Acid Red 50 | 45220 | red |
| Phenyl-2-oxyfluorone-2-carboxylic acid | 45350 | yellow |
| 4,5-Dibromofluorescein | 45370 | orange |
| 2,4,5,7-Tetrabromofluorescein | 45380 | red |
| Solvent Dye | 45396 | orange |
| Acid Red 98 | 45405 | red |
| 3′,4′,5′,6′-Tetrachloro-2,4,5,7-tetrabromofluorescein | 45410 | red |
| 4,5-Diiodofluorescein | 45425 | red |
| 2,4,5,7-Tetraiodofluorescein | 45430 | red |
| Quinophthalone | 47000 | yellow |
| Quinophthalonedisulfonic acid | 47005 | yellow |
| Acid Violet 50 | 50325 | violet |
| Acid Black 2 | 50420 | black |
| Pigment Violet 23 | 51319 | violet |
| 1,2-Dioxyanthraquinone, calcium-aluminum complex | 58000 | red |
| 3-Oxypyrene-5,8,10-sulfonic acid | 59040 | green |
| 1-Hydroxy-4-N-phenylaminoanthraquinone | 60724 | violet |
| 1-Hydroxy-4-(4′-methylphenylamino)anthraquinone | 60725 | violet |
| Acid Violet 23 | 60730 | violet |
| 1,4-Di(4′-methylphenylamino)anthraquinone | 61565 | green |
| 1,4-Bis(o-sulfo-p-toluidino)anthraquinone | 61570 | green |
| Acid Blue 80 | 61585 | blue |
| Acid Blue 62 | 62045 | blue |
| N,N′-Dihydro-1,2,1′,2′-anthraquinonazine | 69825 | blue |
| Vat Blue 6; Pigment Blue 64 | 69825 | blue |
| Vat Orange 7 | 71105 | orange |
| Indigo | 73000 | blue |
| Indigo-disulfonic acid | 73015 | blue |
| 4,4′-Dimethyl-6,6′-dichlorothioindigo | 73360 | red |
| 5,5′-Dichloro-7,7′-dimethylthioindigo | 73385 | violet |
| Quinacridone Violet 19 | 73900 | violet |
| Pigment Red 122 | 73915 | red |
| Pigment Blue 16 | 74100 | blue |
| Phthalocyanine | 74160 | blue |
| Direct Blue 86 | 74180 | blue |
| Chlorinated phthalocyanines | 74260 | green |
| Natural Yellow 6,19; Natural Red 1 | 75100 | yellow |
| Bixin, Norbixin | 75120 | orange |
| Lycopene | 75125 | yellow |
| trans-alpha-, beta- or gamma-Carotene | 75130 | orange |
| Keto and/or hydroxyl derivatives of carotene | 75135 | yellow |
| Guanine or pearlizing agent | 75170 | white |
| 1,7-Bis(4-hydroxy-3-methoxyphenyl)-1,6-heptadiene-3,5-dione | 75300 | yellow |
| Complex salt (Na, Al, Ca) of carminic acid | 75470 | red |
| Chlorophyll a and b; copper compounds of the chlorophylls and | 75810 | green |
| chlorophyllins | | |
| Aluminum | 77000 | white |
| Clay earth hydrate | 77002 | white |
| Hydrous aluminum silicates | 77002 | white |
| Ultramarine | 77007 | blue |
| Pigment Red 101 and 102 | 77015 | red |
| Barium sulfate | 77120 | white |
| Bismuth oxychloride and its mixtures with mica | 77163 | white |
| Calcium carbonate | 77220 | white |
| Calcium sulfate | 77231 | white |
| Carbon | 77266 | black |
| Pigment Black 9 | 77267 | black |
| Carbo medicinalis vegetabilis | 77268 | black |
| Chromium oxide | 77288 | green |
| Chromium oxide, hydrous | 77289 | green |
| Pigment Blue 28, Pigment Green 14 | 77346 | green |
| Pigment Metal 2 | 77400 | brown |
| Gold | 77480 | brown |
| Iron oxides and hydroxides | 77489 | orange |
| Iron oxide | 77491 | red |
| Iron oxide hydrate | 77492 | yellow |
| Iron oxide | 77499 | black |
| Mixtures of iron(II) and iron(III) hexacyanoferrate | 77510 | blue |
| Pigment White 18 | 77713 | white |
| Manganese ammonium diphosphate | 77742 | violet |
| Manganese phosphate; Mn₃(PO₄)₂ 7H2O | 77745 | red |
| Silver | 77820 | white |
| Titanium dioxide and its mixtures with mica | 77891 | white |
| Zinc oxide | 77947 | white |
| 6,7-Dimethyl-9-(1′-D-ribityl)isoalloxazine, lactoflavin | | yellow |
| Sugar coloring | | brown |
| Capsanthin, capsorubin | | orange |
| Betanin | | red |
| Benzopyrylium salts, anthocyans | | red |
| Aluminum, zinc, magnesium and calcium stearate | | white |
| Bromothymol blue | | blue |
| Bromocresol green | | green |
| Acid Red 195 | | red |

Food dyes can also be highly suitable as dyes.

The effector molecules are joined to the resilin protein. The bond between effector molecule and protein can either be a covalent bond, or be based on ionic or van der Waals' interactions or hydrophobic interactions or hydrogen bridge bonds or adsorption.

The covalent linking of the effector molecule can take place via the side chains of the polypeptide sequence of the resilin protein, in particular via amino functions or hydroxy functions or carboxylate functions or thiol functions. Preferably, the linkage is via the amino functions of one or more lysine radicals, via the carboxylate functions of one or more glutamate or aspartate radicals, via the thiol function of one or more cysteine radicals or via the N-terminal or C-terminal function of the resilin protein. Apart from the amino acid functions occurring in the resilin protein sequence, it is also possible for amino acid with suitable functions (e.g. cysteines, lysines, aspartates, glutamates) to be attached to the sequence or to be introduced into the sequence, or for amino acids of the resilin protein to be substituted by such amino acid functions.

The linking of the effector molecules with the resilin protein can take place directly, i.e. as covalent linking of two chemical functions already present. Here, the above-described chemical functions of the resilin proteins are linked with reactive groups which are present in the effector molecules. Examples of such reactive groups are sulfhydryl-reactive groups, e.g. maleimides, pydridyldisulfides, α-haloacetyls, vinylsulfones, sulfatoalkylsulfones (preferably sulfatoethylsulfones), amine-reactive groups (e.g. succinimidyl esters, carbodiimides, hydroxymethylphosphine, imido esters, PFP esters, aldehyde, isothiocyanate etc.), carboxy-reactive groups (e.g. amines etc.), hydroxyl-reactive groups (e.g. isocyanates etc.), unselective groups (e.g. arylazides etc.) and photoactivatable groups (e.g. perfluorophenylazide etc.).

If the functions of resilin protein and effector to be coupled have too low a reactivity for a direct coupling, then these functions can be activated using methods known to the person skilled in the art (e.g. activation of carboxyl functions with carbodiimides).

The linking can, however, also take place via a so-called linker, i.e. an at least bifunctional molecule which enters into a bond with the effector molecule with one or more functions and is linked to the resilin protein with one or more other functions. The use of such tailored linkers permits the precise matching of the linking to the desired effector molecule. Furthermore, it is thereby possible to link a plurality of effector molecules with a resilin protein in a defined way.

The linker used is governed by the functionality to be coupled. Of suitability are, for example, molecules which couple to resilin proteins by means of sulfhydryl-reactive groups, e.g. maleimides, pydridyldisulfides, α-haloacetyls, vinylsulfones, sulfatoalkylsulfones (preferably sulfatoethylsulfones), amine-reactive groups (e.g. succinimidyl esters, carbodiimides, hydroxymethylphosphine, imido esters, PFP esters, aldehyde, isothiocyanate etc.), carboxy-reactive groups (e.g. amines etc.), hydroxyl-reactive groups (e.g. isocyanates etc.), unselective groups (e.g. arylazides etc.) and photoactivatable groups (e.g. perfluorophenylazide etc.) and to effector molecules by means of
sulfhydryl-reactive groups (e.g. maleimides, pydridyldisulfides, α-haloacetyls, vinylsulfones, sulfatoalkylsulfones (preferably sulfatoethylsulfones)
amine-reactive groups (e.g. succinimidyl esters, carbodiimides, hydroxy-methylphosphine, imido esters, PFP esters, aldehyde, isothiocyanate etc.)
sugars and oxidized sugar-reactive groups (e.g. hydrazides etc.)
carboxy-reactive groups (e.g. amines etc.)
hydroxyl-reactive groups (e.g. isocyanates etc.)
thymine-reactive groups (e.g. psoralene etc.)
unselective groups (e.g. aryl azides etc.)
photoactivatable groups (e.g. perfluorophenyl azide etc.)
metal-complexing groups (e.g. EDTA, hexahis, ferritin)
antibodies and fragments (e.g. single-chain antibodies, F(ab) fragments of antibodies, catalytic antibodies).

The chemical functions of a linker can be joined by spacer elements. Spacer elements can be composed, for example, of alkyl chains, ethylene glycol and polyethylene glycols.

Particular preference is given to linker and/or spacer elements which have a potential cleavage site for a protease, lipase, esterase, phosphatase, hydrolase, i.e. are enzymatically cleavable.

Examples of enzymatically cleavable linkers which can be used in the case of the molecules according to the invention are specified, for example, in WO 98/01406, to the entire content of which reference is hereby expressly made.

Particular preference is given to linkers and spacers which are thermocleavable, photocleavable. Corresponding chemical structures are known to the person skilled in the art and are integrated between the molecule parts effector molecule and resilin protein.

If the effector molecule consists of a polypeptide sequence, the linking of effector and resilin protein can take place as a so-called fusion protein, i.e. a general polypeptide sequence is used which is composed of effector and resilin protein part sequences. It is also possible for so-called spacer elements to be incorporated between effector and resilin protein, for example polypeptide sequences which have a potential cleavage site for a protease, lipase, esterase, phosphatase, hydrolase, or oligo- or polypeptide sequences which permit simple purification of the fusion protein, for example so-called His tags, i.e. oligohistidine radicals.

The effector molecules coupled to resilin proteins covalently or noncovalently may be active in their bonded form. Alternatively, the effector molecules couple to resilin proteins can, however, also be released from the resilin proteins.

The release of covalently coupled effector molecules from the resilin proteins can take place through cleavage of specifically introduced cleavable spacers or coupling linkers, which may, for example, be thermocleavable, photocleavable or enzymatically cleavable, but also through proteolytic degradation (e.g. by proteases).

For the use according to the invention in cosmetics, the resilin proteins are formulated with customary further active ingredients and auxiliaries used in cosmetics.

Preferably, the resilin proteins according to the invention are used for skin cosmetics.

Suitable auxiliaries and additives for producing hair cosmetic, nail cosmetic or skin cosmetic preparations are known to the person skilled in the art and can be found in cosmetics handbooks, for example Schrader, Grundlagen und Rezepturen der Kosmetika [Fundamentals and formulations of cosmetics], Hüthig Verlag, Heidelberg, 1989, ISBN 3-7785-1491-1.

The cosmetic compositions according to the invention may be skin cosmetic, nail cosmetic, hair cosmetic, dermatological, hygiene or pharmaceutical compositions.

Preferably, the compositions according to the invention are in the form of a gel, foam, spray, ointment, cream, emulsion, suspension, lotion, milk or paste. If desired, liposomes or microspheres can also be used.

The cosmetically or pharmaceutically active compositions according to the invention can additionally comprise cosmetically and/or dermatologically active ingredients, and auxiliaries.

Preferably, the cosmetic compositions according to the invention comprise at least one resilin protein, and at least one constituent different therefrom which is chosen from cosmetically active ingredients, emulsifiers, surfactants, preservatives, perfume oils, thickeners, hair polymers, hair and skin conditioners, graft polymers, water-soluble or dispersible silicone-containing polymers, photoprotective agents, bleaches, gel formers, care agents, colorants, tinting agents, tanning agents, dyes, pigments, consistency regulators, humectants, refatting agents, collagen, protein hydrolyzates, lipids, antioxidants, antifoams, antistats, emollients and softeners. The resilin proteins can also be present in the cosmetic preparations in encapsulated form.

Advantageously, the antioxidants are chosen from the group consisting of amino acids (e.g. glycine, histidine, tyrosine, tryptophan) and derivatives thereof, imidazoles (e.g. urocanic acid) and derivatives thereof, peptides such as D,L-carnosine, D-carnosine, L-carnosine and derivatives thereof (e.g. anserine), carotenoids, carotenes (e.g. β-carotene, lycopene) and derivatives thereof, chlorogenic acid and derivatives thereof, lipoic acid and derivatives thereof (e.g. dihydrolipoic acid), aurothioglucose, propyl-thiouracil and other thiols (e.g. thioredoxin, glutathione, cysteine, cystine, cystamine and the glycosyl, N-acetyl, methyl, ethyl, propyl, amyl, butyl and lauryl, palmitoyl, oleyl, γ-linoleyl, cholesteryl and glyceryl esters thereof and salts thereof, dilauryl thio-dipropionate, distearyl thiodipropionate, thiodipropionic acid and derivatives thereof (esters, ethers, peptides, lipids, nucleotides, nucleosides and salts), and sulfoximine compounds (e.g. buthionine sulfoximines, homocysteine sulfoximines, buthionine sulfones, penta-, hexa-, heptathionine sulfoximine) in very low tolerated doses (e.g. pmol to μmol/kg), also (metal) chelating agents (e.g. α-hydroxy fatty acids, palmitic acid, phytic acid, lactoferrin), α-hydroxy acids (e.g. citric acid, lactic acid, malic acid), humic acid, bile acid, bile extracts, bilirubin, biliverdin, EDTA and derivatives thereof, unsaturated fatty acids and derivatives thereof (e.g. γ-linolenic acid, linoleic acid, oleic acid), folic acid and derivatives thereof, ubiquinone and ubiquinol and derivatives thereof, vitamin C and derivatives thereof (e.g. sodium ascorbate, ascorbyl palmitate, Mg ascorbyl phosphate, ascorbyl acetate), tocopherol and derivatives (e.g. vitamin E acetate, tocotrienol), vitamin A and derivatives (vitamin A palmitate), an coniferyl benzoate of benzoin resin, rutinic acid and derivatives thereof, α-glycosylrutin, ferulic acid, furfurylideneglucitol, carnosine, butylhydroxytoluene, butylhydroxyanisole, nordihydroguaiacic acid, nordihydroguaiaretic acid, trihydroxybutyrophenone, uric acid and derivatives thereof, mannose and derivatives thereof, zinc and derivatives thereof (e.g. ZnO, ZnSO₄), selenium and derivatives thereof (e.g. selenomethionine), stilbenes and derivatives thereof (e.g. stilbene oxide, trans-stilbene oxide).

Also advantageous are so-called peroxide decomposers, i.e. compound which are able to decompose peroxides, particularly preferably lipid peroxides. These are understood as meaning organic substances, such as, for example, pyridine-2-thiol-3-carboxylic acid, 2-methoxypyrimidinolcarboxylic acids, 2-methoxypyridinecarboxylic acids, 2-dimethylaminopyrimidinolcarboxylic acids, 2-dimethylaminopyridinecarboxylic acids.

Customary thickeners in such formulations are crosslinked polyacrylic acids and derivatives thereof, polysaccharides and derivatives thereof, such as xanthan gum, agar-agar, alginates or tyloses, cellulose derivatives, e.g. carboxymethylcellulose or hydroxycarboxymethylcellulose, fatty alcohols, monoglycerides and fatty acids, polyvinyl alcohol and polyvinylpyrrolidone. Preference is given to using nonionic thickeners.

Suitable cosmetically and/or dermocosmetically active ingredients are, for example, coloring active ingredients, skin and hair pigmentation agents, tinting agents, tanning agents, bleaches, keratin-hardening substances, antimicrobial active ingredients, photofilter active ingredients, repellent active ingredients, hyperemic substances, keratolytically and keratoplastically effective substances, antidandruff active ingredients, antiphlogistics, keratinizing substances, antioxidative active ingredients and/or active ingredients which act as free-radical scavengers, skin moisturizing or humectant substances, refatting active ingredients, antierythematous or antiallergic active ingredients, branched fatty acids, such as 18-methyleicosanoic acid, and mixtures thereof.

Artificially skin-tanning active ingredients which are suitable for tanning the skin without natural or artificial irradiation with UV rays are, for example, dihydroxyacetone, alloxan and walnut shell extract. Suitable keratin-hardening substances are usually active ingredients, as are also used in antiperspirants, such as, for example, potassium aluminum sulfate, aluminum hydroxychloride, aluminum lactate, etc.

Antimicrobial active ingredients are used to destroy microorganisms or to inhibit their growth and thus serve both as preservative and as deodorizing substance which reduces the formation or the intensity of body odor. These include, for example, customary preservatives known to the person skilled in the art, such as p-hydroxy-benzoic esters, imidazolidinylurea, formaldehyde, sorbic acid, benzoic acid, salicylic acid, etc. Such deodorizing substances are, for example, zinc ricinoleate, triclosan, undecylenic acid alkylolamides, triethyl citrate, chlorhexidine etc.

Suitable preservatives, which are listed below with their E number, are to be used advantageously according to the invention.

| | |
|---|---|
| E 200 | Sorbic acid |
| E 201 | Sodium sorbate |
| E 202 | Potassium sorbate |
| E 203 | Calcium sorbate |
| E 210 | Benzoic acid |
| E 211 | Sodium benzoate |
| E 212 | Potassium benzoate |
| E 213 | Calcium benzoate |
| E 214 | Ethyl p-hydroxybenzoate |
| E 215 | Ethyl p-hydroxybenzoate Na salt |
| E 216 | n-Propyl p-hydroxybenzoate |
| E 217 | n-Propyl p-hydroxybenzoate Na salt |
| E 218 | Methyl p-hydroxybenzoate |
| E 219 | Methyl p-hydroxybenzoate Na salt |
| E 220 | Sulfur dioxide |
| E 221 | Sodium sulfite |
| E 222 | Sodium hydrogensulfite |
| E 223 | Sodium disulfite |
| E 224 | Potassium disulfite |
| E 226 | Calcium sulfite |
| E 227 | Calcium hydrogensulfite |
| E 228 | Potassium hydrogensulfite |
| E 230 | Biphenyl (diphenyl) |
| E 231 | Orthophenylphenol |
| E 232 | Sodium orthophenylphenoxide |
| E 233 | Thiabendazole |
| E 235 | Natamycin |
| E 236 | Formic acid |
| E 237 | Sodium formate |
| E 238 | Calcium formate |
| E 239 | Hexamethylenetetramine |
| E 249 | Potassium nitrite |
| E 250 | Sodium nitrite |
| E 251 | Sodium nitrate |
| E 252 | Potassium nitrate |
| E 280 | Propionic acid |
| E 281 | Sodium propionate |
| E 282 | Calcium propionate |
| E 283 | Potassium propionate |
| E 290 | Carbon dioxide |

Also suitable according to the invention are preservatives or preservative auxiliaries customary in cosmetics dibromodicyanobutane (2-bromo-2-bromomethyl-glutarodinitrile), 3-iodo-2-propynyl butylcarbamate, 2-bromo-2-nitropropane-1,3-diol, imidazolidinylurea, 5-chloro-2-methyl-4-isothiazolin-3-one, 2-chloroacetamide, benzalkonium chloride, benzyl alcohol, formaldehyde cleavers.

Also suitable as preservatives are phenyl hydroxyalkyl ethers, in particular the compound known under the name phenoxyethanol on account of its bactericidal and fungicidal effects on a number of microorganisms.

Other antimicrobial agents are likewise suitable for being incorporated into the preparations according to the invention. Advantageous substances are, for example, 2,4,4′-trichloro-2′-hydroxydiphenyl ether (irgasan), 1,6-di(4-chlorophenylbiguanido)-hexane (chlorhexidine), 3,4,4′-trichlorocarbanilide, quaternary ammonium compounds, oil of cloves, mint oil, thyme oil, triethyl citrate, farnesol (3,7,11-trimethyl-2,6,10-dodecatrien-1-ol), and the active ingredients or active ingredient combinations described in the patent laid-open specifications DE-37 40 186, DE-39 38 140, DE-42 04 321, DE-42 29 707, DE-43 09 372, DE-44 11 664, DE-195 41 967, DE-195 43695, DE-195 43696, DE-195 47160, DE-196 02 108, DE-196 02 110, DE-196 02 111, DE-1 96 31 003, DE-1 96 31 004 and DE-1 96 34 019 and the patent specifications DE-42 29 737, DE-42 37 081, DE-43 24 219, DE-44 29 467, DE-44 23 410 and DE-195 16 705. Sodium hydrogencarbonate is also to be used advantageously.

Suitable photofilter active ingredients are substances which absorb UV rays in the UV-B and/or UV-A region. Suitable UV filters are, for example, 2,4,6-triaryl-1,3,5-triazines in which the aryl groups can each carry at least one substituent which is preferably chosen from hydroxy, alkoxy, specifically methoxy, alkoxycarbonyl, specifically methoxycarbonyl and ethoxycarbonyl and mixtures thereof. Also suitable are p-aminobenzoic esters, cinnamic esters, benzophenones, camphor derivatives, and pigments which stop UV rays, such as titanium dioxide, talc and zinc oxide.

Suitable UV filter substances are any UV-A and UV-B filter substances. Examples to be mentioned are:

| | | CAS No. |
|---|---|---|
| No. | Substance | (=acid) |
| 1 | 4-Aminobenzoic acid | 150-13-0 |
| 2 | 3-(4′-Trimethylammonium)benzylidenebornan-2-one methyl | 52793-97-2 |
| | sulfate | |
| 3 | 3,3,5-Trimethylcyclohexyl salicylate | 118-56-9 |
| | (homosalate) | |
| 4 | 2-Hydroxy-4-methoxybenzophenone | 131-57-7 |
| | (oxybenzone) | |
| 5 | 2-Phenylbenzimidazole-5-sulfonic acid and its potassium, | 27503-81-7 |
| | sodium and triethanolamine salts | |
| 6 | 3,3′-(1,4-Phenylenedimethine)bis(7,7-dimethyl- | 90457-82-2 |
| | 2-oxobicyclo[2.2.1]heptane-1-methanesulfonic acid) and its | |
| | salts | |
| 7 | Polyethoxyethyl 4-bis(polyethoxy)aminobenzoate | 113010-52-9 |
| 8 | 2-Ethylhexyl 4-dimethylaminobenzoate | 21245-02-3 |
| 9 | 2-Ethylhexyl salicylate | 118-60-5 |
| 10 | 2-Isoamyl 4-methoxycinnamate | 71617-10-2 |
| 11 | 2-Ethylhexyl 4-methoxycinnamate | 5466-77-3 |
| 12 | 2-Hydroxy-4-methoxybenzophenone-5-sulfonic acid | 4065-45-6 |
| | (sulisobenzone) and the sodium salt | |
| 13 | 3-(4′-Sulfobenzylidene)bornan-2-one and salts | 58030-58-6 |
| 14 | 3-Benzylidenebornan-2-one | 16087-24-8 |
| 15 | 1-(4′-Isopropylphenyl)-3-phenylpropane-1,3-dione | 63260-25-9 |
| 16 | 4-Isopropylbenzyl salicylate | 94134-93-7 |
| 17 | 3-Imidazol-4-ylacrylic acid and its ethyl ester | 104-98-3 |
| 18 | Ethyl 2-cyano-3,3-diphenylacrylate | 5232-99-5 |
| 19 | 2′-Ethylhexyl 2-cyano-3,3-diphenylacrylate | 6197-30-4 |
| 20 | Menthyl o-aminobenzoate or: | 134-09-8 |
| | 5-methyl-2-(1-methylethyl)-2-aminobenzoate | |
| 21 | Glyceryl p-aminobenzoate or: 1-glyceryl 4-aminobenzoate | 136-44-7 |
| 22 | 2,2′-Dihydroxy-4-methoxybenzophenone (dioxybenzone) | 131-53-3 |
| 23 | 2-Hydroxy-4-methoxy-4-methylbenzophenone | 1641-17-4 |
| | (mexenone) | |
| 24 | Triethanolamine salicylate | 2174-16-5 |
| 25 | Dimethoxyphenylglyoxalic acid or: | 4732-70-1 |
| | 3,4-dimethoxyphenylglyoxal acidic sodium | |
| 26 | 3-(4′-Sulfobenzylidene)bornan-2-one and its salts | 56039-58-8 |
| 27 | 4-tert-Butyl-4′-methoxydibenzoylmethane | 70356-09-1 |
| 28 | 2,2′,4,4′-Tetrahydroxybenzophenone | 131-55-5 |
| 29 | 2,2′-Methylenebis[6-(2H-benzotriazol-2-yl)-4-(1,1,3,3,-tetra- | 103597-45-1 |
| | methylbutyl)phenol] | |
| 30 | 2,2′-(1,4-Phenylene)bis-1H-benzimidazole- | 180898-37-7 |
| | 4,6-disulfonic acid, Na salt | |
| 31 | 2,4-bis[4-(2-Ethylhexyloxy)-2-hydroxy]phenyl- | 187393-00-6 |
| | 6-(4-methoxyphenyl)-(1,3,5)-triazine | |
| 32 | 3-(4-Methylbenzylidene)camphor | 36861-47-9 |
| 33 | Polyethoxyethyl 4-bis(polyethoxy)paraaminobenzoate | 113010-52-9 |
| 34 | 2,4-Dihydroxybenzophenone | 131-56-6 |
| 35 | 2,2′-Dihydroxy-4,4′-dimethoxybenzophenone- | 3121-60-6 |
| | 5,5′-disodium sulfonate | |
| 36 | Benzoic acid, 2-[4-(diethylamino)-2-hydroxybenzoyl], hexyl ester | 302776-68-7 |
| 37 | 2-(2H-Benzotriazol-2-yl)-4-methyl-6-[2-methyl-3-[1,3,3,3-tetra- | 155633-54-8 |
| | methyl-1-[(trimethylsilyl)oxy]disiloxanyl]propyl]phenol | |
| 38 | 1,1-[(2,2′-Dimethylpropxy)carbonyl]-4,4-diphenyl-1,3-butadiene | 363602-15-7 |

The cosmetic and dermatological preparations according to the invention can advantageously also comprise inorganic pigments which stop UV rays and are based on metal oxides and/or other metal compounds which are insoluble or sparingly soluble in water, chosen from the group of oxides of zinc (ZnO), titanium (TiO₂), iron (e.g. Fe₂O₃), zirconium (ZrO₂), silicon (SiO₂), manganese (e.g. MnO), aluminum (Al₂O₃), cerium (e.g. Ce₂O₃), mixed oxides of the corresponding metals and mixtures of such oxides.

The inorganic pigments can be present here in coated form, i.e. are treated superficially. This surface treatment can consist, for example, in providing the pigments with a thin hydrophobic layer by a method known per se, as described in DE-A-33 14 742.

Suitable repellent active ingredients are compounds which are able to repel or drive away certain animals, in particular insects, from humans. These include, for example, 2-ethyl-1,3-hexanediol, N,N-diethyl-m-toluamide etc. Suitable hyperemic substances, which stimulate the flow of blood through the skin, are e.g. essential oils, such as dwarf pine extract, lavender extract, rosemary extract, juniperberry extract, horse chestnut extract, birch leaf extract, hayflower extract, ethyl acetate, camphor, menthol, peppermint oil, rosemary extract, eucalyptus oil, etc. Suitable keratolytic and thioglycolic acid and its salts, sulfur, etc. Suitable antidandruff active ingredients are, for example, sulfur, sulfur polyethylene glycol sorbitan monooleate, sulfur ricinol polyethoxylate, zinc pyrithione, aluminum pyrithione, etc. Suitable antiphlogistics, which counteract skin irritations, are, for example, allantoin, bisabolol, dragosantol, camomile extract, panthenot, etc.

The cosmetic compositions according to the invention can comprise, as cosmetic and/or pharmaceutical active ingredient (also, if appropriate, as auxiliary), at least one cosmetically or pharmaceutically acceptable polymer. These include, quite generally, cationic, amphoteric and neutral polymers.

Suitable polymers are, for example, cationic polymers with the INCI name Polyquaternium, e.g. copolymers of vinylpyrrolidone/N-vinylimidazolium salts (Luviquat FC, Luviquat HM, Luviquat MS, Luviquat&commat, Care), copolymers of N-vinyl-pyrrolidone/dimethylaminoethyl methacrylate, quaternized with diethyl sulfate (Luviquat PQ 11), copolymers of N-vinylcaprolactam/N-vinylpyrrolidone/N-vinylimidazolium salts (Luviquat E Hold), cationic cellulose derivatives (Polyquaternium-4 and -10), acrylamide copolymers (Polyquaternium-7) and chitosan.

Suitable cationic (quaternized) polymers are also Merquat (polymer based on dimethyldiallylammonium chloride), Gafquat (quaternary polymers which are formed by reacting polyvinylpyrrolidone with quaternary ammonium compounds), polymer JR (hydroxyethylcellulose with cationic groups) and plant-based cationic polymers, e.g. guar polymers, such as the Jaguar grades from Rhodia.

Further suitable polymers are also neutral polymers, such as polyvinylpyrrolidones, copolymers of N-vinylpyrrolidone and vinyl acetate and/or vinyl propionate, poly-siloxanes, polyvinylcaprolactam and other copolymers with N-vinylpyrrolidone, polyethyleneimines and salts thereof, polyvinylamines and salts thereof, cellulose derivatives, polyaspartic acid salts and derivatives. These include, for example, Luviflex 0 Swing (partially hydrolyzed copolymer of polyvinyl acetate and polyethylene glycol, BASF).

Suitable polymers are also nonionic, water-soluble or water-dispersible polymers or oligomers, such as polyvinylcaprolactam, e.g. Luviskol 0 Plus (BASF), or polyvinyl-pyrrolidone and copolymers thereof, in particular with vinyl esters, such as vinyl acetate, e.g. Luviskol 0 VA 37 (BASF), polyamides, e.g. based on itaconic acid and aliphatic diamines, as are described, for example, in DE-A-43 33 238,

Suitable polymers are also amphoteric or zwitterionic polymers, such as the octylacrylamide/methyl methacrylate/tert-butylaminoethyl methacrylate-hydroxypropyl methacrylate copolymers obtainable under the names Amphomer (National Starch), and zwitterionic polymers, as are disclosed, for example, in the German patent applications DE39 29 973, DE 21 50 557, DE28 17 369 and DE 3708 45 Acrylamido-propyltrimethylammonium chloride/acrylic acid or methacrylic acid copolymers and alkali metal and ammonium salts thereof are preferred zwitterionic polymers. Further suitable zwitterionic polymers are methacroylethylbetaine/methacrylate polymers, which are commercially available under the name Amersette (AMERCH L), and copolymers of hydroxyethyl methacrylate, methyl methacrylate, N,N-dimethylamino-ethyl methacrylate and acrylic acid (Jordapon (D)).

Suitable polymers are also nonionic, siloxane-containing, water-soluble or dispersible polymers, e.g. polyether siloxanes, such as Tegopren 0 (Goldschmidt) or Besi&commat (Wacker).

The formulation base of cosmetic compositions according to the invention preferably comprises cosmetically and/or pharmaceutically acceptable auxiliaries.

Pharmaceutically acceptable auxiliaries are the auxiliaries which are known for use in the field of pharmacy, food technology and related fields, in particular the auxiliaries listed in the relevant pharmacopeia (e.g. DAB Ph. Eur. BP NF), and other auxiliaries whose properties do not preclude a physiological application.

Suitable auxiliaries may be: glidants, wetting agents, emulsifying and su pending agents, preservatives, antioxidants, antiirritatives, chelating agents, emulsion stabilizers, film formers, gel formers, odor masking agents, resins, hydrocolloids, solvents, solubility promoters, neutralizing agents, permeation accelerators, pigments, quaternary ammonium compounds, refatting and superfatting agents, ointment, cream or oil base substances, silicone derivatives, stabilizers, sterilizing agents, propellants, drying agents, opacifiers, thickeners, waxes, softeners, white oil. An embodiment in this regard is based on specialist knowledge, as shown, for example, in Fiedler, H. P. Lexikon der Hilfsstoffe für Pharmazie, Kosmetik und angrenzende Gebiete [Lexicon of auxiliaries for pharmacy, cosmetics and related fields], 4th edition, Aulendorf: ECV-Editio-Kantor-Verlag, 1996.

To produce the dermocosmetic compositions according to the invention, the active ingredients can be mixed or diluted with a suitable auxiliary (excipient). Excipients may be solid, semisolid or liquid materials which can serve as vehicles, carriers or medium for the active ingredient. The admixing of further auxiliaries takes place, if desired, in the manner known to the person skilled in the art. In addition, the polymers and dispersions are suitable as auxiliaries in pharmacy, preferably as or in (a) coating composition(s) or binder(s) for solid drug forms. They can also be used in creams and as tablet coatings and tablet binders.

According to a preferred embodiment, the compositions according to the invention are a skin cleansing composition.

Preferred skin cleansing compositions are soaps of liquid to gel-like consistency, such as transparent soaps, luxury soaps, deodorant soaps, cream soaps, baby soaps, skin protection soaps, abrasive soaps and syndets, pasty soaps, soft soaps and washing pastes, exfoliating soaps, moisture wipes, liquid washing, showering and bathing preparations, such as washing lotions, shower baths and gels, foam baths, oil baths and scrub preparations, shaving foams, lotions and creams.

According to a further preferred embodiment, the compositions according to the invention are cosmetic compositions for the care and protection of the skin and hair, nailcare compositions or preparations for decorative cosmetics.

Suitable skin cosmetic compositions are, for example, face tonics, face asks, deodorants and other cosmetic lotions. Compositions for use in decorative cosmetics include, for example, concealing sticks, stage make-up, mascara and eye shadows, lipsticks, kohl pencils, eyeliners, blushers, powders and eyebrow pencils.

Furthermore, the dermatological compositions according to the invention can be used in nose strips for pore cleansing, in antiacne compositions, repellents, shaving compositions, aftershave and preshave care compositions, aftersun care compositions, hair removal compositions, hair colorants, intimate care compositions, footcare compositions, and in baby care.

The skincare compositions according to the invention are, in particular, W/O or O/W skin creams, day creams and night creams, eye creams, face creams, antiwrinkle creams, sunscreen creams, moisturizing creams, bleaching creams, self-tanning creams, vitamin creams, skin lotions, care lotions and moisturizing lotions.

Skin cosmetic and dermatological compositions based on the resilin proteins described above display advantageous effects. The resilin proteins can, inter alia, contribute to the moisturization and conditioning of the skin and to an improvement in the feel of the skin. The resilin proteins can also act as thickeners in the formulations. By adding the resilin proteins according to the invention it is possible, in certain formulations, to achieve a considerable improvement in skin compatibility.

Skin cosmetic and dermatological compositions preferably comprise at least one resilin protein in an amount of from about 0.001 to 30% by weight, preferably 0.1 to 20% by weight, very particularly preferably 0.1 to 12% by weight, based on the to a weight of the composition.

Particularly photoprotective compositions based on the resilin proteins have the property of increasing the residence time of the UV-absorbing ingredients compared to customary auxiliaries such as polyvinylpyrrolidone.

Depending on the field of use, the compositions according to the invention can be applied in a form suitable for skincare, such as, for example, as cream, foam, gel, stick, mousse, milk, spray (pump spray or propellant-containing spray) or lotion.

Besides the resilin proteins and suitable carriers, the skin cosmetic preparations can also comprise further active ingredients and auxiliaries customary in skin cosmetics, as described above. These include, preferably, emulsifiers, preservatives, perfume oils, cosmetic active ingredients, such as phytantriol, vitamin A, E and C, retinol, bisabolol, panthenol, photoprotective agents, bleaches, colorants, tinting agents, tanning agents, collagen, enzymes, protein hydrolyzates, stabilizers, pH regulators, dyes, salts, thickeners, gel formers, consistency regulators, silicones, humectants, refatting agents and/or further customary additives.

Preferred oil and fat components of the skin cosmetic and dermatological compositions are the abovementioned mineral and synthetic oils, such as, for example, paraffins, silicone oils and aliphatic hydrocarbons having more than 8 carbon atoms, animal and vegetable oils, such as, for example, sunflower oil, coconut oil, avocado oil, olive oil, lanolin, or waxes, fatty acids, fatty acid esters, such as, for example, triglycerides of C6-C30 fatty acids, wax esters, such as, for example, jojoba oil, fatty alcohols, vaseline, hydrogenated lanolin and acetylated lanolin, and mixtures thereof.

The resilin proteins according to the invention can also be mixed with conventional polymers if specific properties are to be established.

To establish certain properties, such as, for example, improving the feel to the touch, the spreading behavior, the water resistance and/or the binding of active ingredients and auxiliaries such as pigments, the skin cosmetic and dermatological preparations can additionally also comprise conditioning substances based on silicone compounds.

Suitable silicone compounds are, for example, polyalkylsiloxanes, polyarylsiloxanes, polyarylalkylsiloxanes, polyether siloxanes or silicone resins.

The cosmetic or dermocosmetic preparations are produced by customary methods known to the person skilled in the art.

Preferably, the cosmetic and dermocosmetic compositions are present in the form of emulsions, in particular as water-in-oil (W/O) or oil-in-water (O/W) emulsions.

However, it is also possible to choose other types of formulation, for example gels, oils, oleogels, multiple emulsions, for example in the form of W/O/W or O/W/O emulsions, anhydrous ointments or ointment bases, etc. Emulsifier-free formulations, such as hydrodispersions, hydrogels or a Pickering emulsion are also advantageous embodiments.

Emulsions are produced by known methods. Besides at least one resilin protein, the emulsions usually comprise customary constituents, such as fatty alcohols, fatty acid esters and, in particular, fatty acid triglycerides, fatty acids, lanolin and derivatives thereof, natural or synthetic oils or waxes and emulsifiers in the presence of water. The choice of additives specific to the type of emulsion and the production of suitable emulsions is described, for example, in Schrader, Grundlagen und Rezepturen der Kosmetika [Fundamentals and formulations of cosmetics], Hüthig Buch Verlag, Heidelberg, 2nd edition, 1989, third part, to which reference is hereby expressly made.

A suitable emulsion in the form of a W/O emulsion, e.g. for a skin cream etc., generally comprises an aqueous phase which is emulsified in an oil or fatty phase using a suitable emulsifier system. A polyelectrolyte complex can be used for the provision of the aqueous phase.

Preferred fatty components which may be present in the fatty phase of the emulsions are: hydrocarbon oils, such as paraffin oil, purcellin oil, perhydrosqualene and solutions of microcrystalline waxes in these oils; animal or vegetable oils, such as sweet almond oil, avocado oil, calophyllum oil, lanolin and derivatives thereof, castor oil, sesame oil, olive oil, jojoba oil, karite oil, hoplostethus oil, mineral oils whose distillation start-point under atmospheric pressure is at about 250° C. and whose distillation end-point is at 410° C., such as, for example, Vaseline oil, esters of saturated or unsaturated fatty acids, such as alkyl myristates, e.g. isopropyl myristate, butyl myristate or cetyl myristate, hexadecyl stearate, ethyl or isopropyl palmitate, octanoic or decanoic acid triglycerides and cetyl ricinoleate.

The fatty phase can also comprise silicone oils which are soluble in other oils, such as dimethylpolysiloxane, methylphenylpolysiloxane and the silicone glycol copolymer, fatty acids and fatty alcohols.

Besides the resilin proteins, waxes can also be used, such as, for example, carnauba wax, candelilla wax, beeswax, microcrystalline wax, ozokerite wax and Ca, Mg and Al oleates, myristates, linoleates and stearates.

In addition, an emulsion according to the invention may be in the form of an O/W emulsion. Such an emulsion usually comprises an oil phase, emulsifiers which stabilize the oil phase in the water phase, and an aqueous phase, which is usually present in thickened form. Suitable emulsifiers are preferably O/W emulsifiers, such as polyglycerol esters, sorbitan esters or partially esterified glycerides.

According to a further preferred embodiment, the compositions according to the invention are a shower gel, a shampoo formulation or a bathing preparation.

Such formulations comprise at least one resilin protein and usually anionic surfactants as base surfactants and amphoteric and/or nonionic surfactants as cosurfactants. Further suitable active ingredients and/or auxiliaries are generally chosen from lipids, perfume oils, dyes, organic acids, preservatives and antioxidants, and thickeners/gel formers, skin conditioning agents and humectants.

These formulations advantageously comprise 2 to 50% by weight, preferably 5 to 40% by weight, particularly preferably 8 to 30% by weight, of surfactants, based on the total weight of the formulation.

In the washing, shower and bath preparations, all of the anionic, neutral, amphoteric or cationic surfactants customarily used in body-cleansing compositions can be used.

Suitable anionic surfactants are, for example, alkyl sulfates, alkyl ether sulfates, alkylsulfonates, alkylarylsulfonates, alkyl succinates, alkyl sulfosuccinates, N-alkoyl sarcosinates, acyl taurates, acyl isethionates, alkyl phosphates, alkyl ether phosphates, alkyl ether carboxylates, alpha-olefinsulfonates, in particular the alkali metal and alkaline earth metal salts, e.g. sodium, potassium, magnesium, calcium, and ammonium and triethanolamine salts. The alkyl ether sulfates, alkyl ether phosphates and alkyl ether carboxylates can have between 1 and 10 ethylene oxide r propylene oxide units, preferably 1 to 3 ethylene oxide units, in the molecule.

These include, for example, sodium lauryl sulfate, ammonium lauryl sulfate, sodium lauryl ether sulfate, ammonium lauryl ether sulfate, sodium lauryl sarcosinate, sodium oleyl succinate, ammonium lauryl sulfosuccinate, sodium dodecylbenzenesulfonate, triethanolamine dodecylbenzenesulfonate.

Suitable amphoteric surfactants are, for example, alkylbetaines, alkylamidopropylbetaines, alkylsulfobetaines, alkyl glycinates, alkyl carboxyglycinates, alkyl amphoacetates or -propionates, alkyl amphodiacetates or -dipropionate

For example, cocodimethylsulfopropylbetaine, laurylbetaine, cocamidopr opylbetaine or sodium cocamphopropionate can be used.

Suitable nonionic surfactants are, for example, the reaction products of aliphatic alcohols or alkylphenols having 6 to 20 carbon atoms in the alkyl chain, which may be linear or branched, with ethylene oxide and/or propylene oxide. The amount of alkylene oxide is about 6 to 60 mol per mole of alcohol. In addition, alkylamine oxides, mono or dialkylalkanolamides, fatty acid esters of polyethylene glycols, ethoxylated fatty acid amides, alkyl polyglycosides or sorbitan ether esters are suitable.

Furthermore, the washing, shower and bath preparations can comprise customary cationic surfactants, such as, for example, quaternary ammonium compounds, for example cetyltrimethylammonium chloride.

In addition, the shower gel/shampoo formulations can comprise thickeners, such as, for example, sodium chloride, PEG-55, propylene glycol oleate, PEG-120 methylglucose dioleate and others, and also preservatives, further active ingredients and auxiliaries and water.

According to a further preferred embodiment, the compositions according to the invention are a hair-treatment composition.

Hair-treatment compositions according to the invention preferably comprise at least one resilin protein in an amount in the range from about 0.01 to 30% by weight, preferably 0.5 to 20% by weight, based on the total weight of the composition.

Preferably, the hair treatment compositions according to the invention are in the form of a setting foam, hair mousse, hair gel, shampoo, hair spray, hair foam, end fluid, neutralizer for permanent waves, hair colorant and bleach or hot-oil treatment. Depending on the field of use, the hair cosmetic preparations can be applied as (aerosol) spray, (aerosol) foam, gel, gel spray, cream, lotion or wax. Hair sprays include here both aerosol sprays and also pump sprays without propellant gas. Hair foams include both aerosol foams and also pump foams without propellant gas. Hair sprays and hair foams preferably include predominantly or exclusively ater-soluble or water-dispersible components. If the compounds used in the hair spray and hair foams according to the invention are dispersible in water, they can be a plied in the form of aqueous microdispersions with particle diameters of usually 1 to 350 nm, preferably 1 to 250 nm. The solids contents of these preparations are here usually in a range from about 0.5 to 20% by weight. These microdispersions do not usually require emulsifiers or surfactants for their stabilization.

The hair cosmetic formulations according to the invention comprise, in a preferred embodiment, a) 0.01 to 30% by weight of at least one resilin protein, b) 20 to 99.95% by weight of water and/or alcohol, c) 0 to 50% by weight of at least one propellant gas, d) 0 to 5% by weight of at least one emulsifier, e) 0 to 3% by weight of at least one thickener, and up to 25% by weight of further constituents.

Alcohol is understood as meaning all alcohols customary in cosmetics, e.g. ethanol, isopropanol, n-propanol.

Further constituents are to be understood as meaning the additives customary in cosmetics, for example propellants, antifoams, interface-active compounds, i.e. surfactants, emulsifiers, foam formers and solubilizers. The interface-active compounds used may be anionic, cationic, amphoteric or neutral. Further customary constituents, may also be, for example, preservatives, perfume oils, opacifiers, active ingredients, UV filters, care substances, such as panthenol, collagen, vitamins, protein hydrolyzates, alpha- and beta-hydroxycarboxylic acids, stabilizers, pH regulators, dyes, viscosity regulators, gel formers, salts, humectants, refatting agents, complexing agents and further customary additives.

Also included here are all styling and conditioner polymers known in cosmetics which can be used in combination with the resilin proteins according to the invention if quite specific properties are to be established.

Suitable conventional hair cosmetics polymers are, for example, the abovementioned cationic, anionic, neutral, nonionic and amphoteric polymers, to which reference is made here.

To establish certain properties, the preparations can additionally also comprise conditioning substances based on silicone compounds. Suitable silicon compounds are, for example, polyalkylsiloxanes, polyarylsiloxanes, polyarylalkylsiloxanes, polyether siloxanes, silicone resins or dimethicone copolyols (CTFA) and amino-functional silicone compounds, such as amodimethicones (CTFA).

The polymers according to the invention are particularly suitable as setting compositions in hair styling preparations, in particular hair sprays (aerosol sprays and pump sprays without propellant gas) and hair foams (as aerosol foams and pump foams without propellant gas).

In a preferred embodiment, spray preparations comprise a) 0.01 to 30% by weight of at least one resilin protein, b) 20 to 99.9% by weight of water and/or alcohol, c) 0 to 70% by weight of at least one propellant, d) 0 to 20% by weight of further constituents.

Propellants are the propellants customarily used for hair sprays or aerosol foams. Preference is given to mixtures of propane/butane, pentane, dimethyl ether, 1,1-difluoroethane (HFC-152 a), carbon dioxide, nitrogen or compressed air.

A formulation preferred according to the invention for aerosol hair foams comprises a) 0.01 to 30% by weight of at least one resilin protein, b) 55 to 99.8% by weight of water and/or alcohol, c) 5 to 20% by weight of a propellant, d) 0.1 to 5% by weight of an emulsifier, e) 0 to 10% by weight of further constituents.

Emulsifiers which can be used are all emulsifiers customarily used in hair foams. Suitable emulsifiers may be nonionic, cationic or anionic or amphoteric.

Examples of nonionic emulsifiers (INCI nomenclature) are laureths, e.g. laureth-4; ceteths, e.g. ceteth-1, polyethylene glycol cetyl ether, ceteareths, e.g. ceteareth-25, polyglycol fatty acid glycerides, hydroxylated lecithin, lactyl esters of fatty acids, alkyl polyglycosides.

Examples of cationic emulsifiers are cetyldimethyl-2-hydroxyethylammonium dihydrogenphosphate, cetyltrimonium chloride, cetyltrimonium bromide, cocotrimonium methyl sulfate, Quaternium-1 to x (INCI).

Anionic emulsifiers can be chosen, for example, from the group of alkyl sulfates, alkyl ether sulfates, alkylsulfonates, alkylarylsulfonates, alkyl succinates, alkyl sulfosuccinates, N-alkoyl sarcosinates, acyl taurates, acyl isethionates, alkyl phosphates, alkyl ether phosphates, alkyl ether carboxylates, alpha-olefinsulfonates, in particular the alkali metal and alkaline earth metal salts, e.g. sodium, potassium, magnesium, calcium, and ammonium and triethanolamine salts. The alkyl ether sulfates, alkyl ether phosphates and alkyl ether carboxylates can have between 1 and 10 ethylene oxide or propylene oxide units, preferably 1 to 3 ethylene oxide units, in the molecule.

A preparation suitable according to the invention for styling gels can, for example, have the following composition: a) 0.01 to 30% by weight of at least one resilin protein, b) 80 to 99.85% by weight of water and/or alcohol, c) 0 to 3% by weight, preferably 0.05 to 2% by weight, of a gel former, d) 0 to 20% by weight of further constituents.

The use of gel formers may be advantageous in order to establish specific rheological or other application-related properties of the gels. Gel formers which can be used are all gel formers customary in cosmetics. These include slightly crosslinked polyacrylic acid, for example Carbomer (INCI), cellulose derivatives, e.g. hydroxypropylcellulose, hydroxyethylcellulose, cationically modified celluloses, polysaccharides, e.g. xanthan gum, caprylic/capric triglyceride, sodium acrylate copolymers, polyquaternium-32 (and) Paraffinum Liquidum (INCI), sodium acrylate copolymers (and) paraffinum liquidum (and) PPG-1 trideceth-6, acrylamidopropyltrimonium chloride/acrylamide copolymers steareth-10 allyl ether, acrylate copolymers, polyquaternium-37 (and) paraffinum liquidum (and) PPG-1 trideceth-6, polyquaternium 37 (and) propylene glycol dicaprate dicaprylate (and) PPG-1 trideceth-6, polyquaternium-7, polyquaternium-44.

The resilin proteins according to the invention can be used as conditioners in cosmetic preparations.

A preparation comprising the resilin proteins according to the invention can preferably be used in shampoo formulations as setting and/or conditioning compositions. Preferred shampoo formulations comprise a) 0.01 to 30% by weight of at least one resilin protein, b) 25 to 94.95% by weight of water, c) 5 to 50% by weight of surfactants, c) 0 to 5% by weight of a further conditioning agent, d) 0 to 10% by weight of further cosmetic constituents.

In the shampoo formulations, all of the anionic, neutral, amphoteric or cationic surfactants customarily used in shampoos can be used.

Suitable anionic surfactants are, for example, alkyl sulfates, alkyl ether sulfates, alkylsulfonates, alkylarylsulfonates, alkyl succinates, alkyl sulfosuccinates, N-alkoyl sarcosinates, acyl taurates, acyl isethionates, alkyl phosphates, alkyl ether phosphates, alkyl ether carboxylates, alpha-olefinsulfonates, in particular the alkali metal and alkaline earth metal salts, e.g. sodium, potassium, magnesium, calcium, and ammonium and triethanolamine salts. The alkyl ether sulfates, alkyl ether phosphates and alkyl ether carboxylates can have between 1 and 10 ethylene oxide or propylene oxide units, preferably 1 to 3 ethylene oxide units, in the molecule.

Of suitability are, for example, sodium lauryl sulfate, ammonium lauryl sulfate, sodium lauryl ether sulfate, ammonium lauryl ether sulfate, sodium lauroyl sarcosinate, sodium oleyl succinate, ammonium lauryl sulfosuccinate, sodium dodecylbenzenesulfonate, triethanolamine dodecylbenzenesulfonate.

Suitable amphoteric surfactants are, for example, alkylbetaines, alkylamidopropylbetaines, alkylsulfobetaines, alkyl glycinates, alkyl carboxyglycinates, alkyl amphoacetates or -propionates, alkyl amphodiacetates or -dipropionates.

For example, cocodimethylsulfopropylbetaine, laurylbetaine, cocamidopropylbetaine or sodium cocamphopropionate can be used.

Suitable nonionic surfactants are, for example, the reaction products of aliphatic alcohols or alkylphenols having 6 to 20 carbon atoms in the alkyl chain, which may be linear or branched, with ethylene oxide and/or propylene oxide. The amount of alkylene oxide is about 6 to 60 mol per mole of alcohol. In addition, alkylamine oxides, mono or dialkylalkanolamides, fatty acid esters of polyethylene glycols, alkyl polyglycosides or sorbitan ether esters are suitable.

Furthermore, the shampoo formulations can comprise customary cationic surfactants, such as, for example, quaternary ammonium compounds, for example cetyltrimethylammonium chloride.

In the shampoo formulations, in order to achieve certain effects, customary conditioning agents can be used in combination with the resilin proteins.

These include, for example, the abovementioned cationic polymers with the INCI name Polyquaternium, in particular copolymers of vinylpyrrolidone/N-vinylimidazolium salts (Luviquat FC, Luviquat&commat, HM, Luviquat MS, Luviquat Care), copolymers of N-vinylpyrrolidone/dimethylaminoethyl methacrylate, quaternized with diethyl sulfate (Luviquat D PQ 11), copolymers of N-vinylcaprolactam/N-vinylpyrrolidone/N-vinyl-imidazolium salts (Luviquat D Hold), cationic cellulose derivatives (Polyquaternium-4 and -10), acrylamide copolymers (Polyquaternium-7). In addition, protein hydrolyzates can be used, and also conditioning substances based on silicone compounds, for example polyalkylsiloxanes, polyarylsiloxanes, polyarylalkylsiloxanes, polyether siloxanes or silicone resins. Further suitable silicone compounds are dimethicone copolyols (CTFA) and amino-functional silicone compounds, such as amodimethicones (CTFA). In addition, cationic guar derivatives, such as Guar Hydroxypropyltrimonium Chloride (INCI) can be used.

The invention further provides the production and use of basic resilins. Basic resilins are ones in which the ratio of the basic amino acid radicals (K, R) present in the repeat units which form the resilin protein to the acidic amino acid radicals (D, E) is at least 1.5, preferably at least 2. Particularly advantageous basic resilins are those in which the ratio of basic amino acid radicals to acidic amino acid radicals at least 40% of the repeat units is 1:1 and at least 40% of the repeat units is 1:0.

Particular preference is given to the production and use of basic resilin proteins which comprise the repeat unit GAPGGGNGGRPSDTY or GAPGGGNGGRPSSSY or functional equivalents of this repeat unit. Particular preference is given to the production and use of the synthetic resilin protein U₁₆ (SEQ ID NO 1) an also of the nucleotide sequence coding therefor (SEQ ID NO 2).

Basic resilin proteins are particularly suitable for use in cosmetics, for the formulation of substances, for the finishing of paper, leather and textiles, for the coating of surfaces and for use in human and animal nutrition.

### Experimental Section

### Example 1

### Production of the Synthetic Resilin Protein U₁₆

### Gene Synthesis and Expression

A synthetic gene which codes for the resilin protein U₁₆, was multimerized by multimerization of a synthetic oligonucleotide U: 5′-ggtgcgccgggcggtggcaacggtggccgtccgtctgacacctacggtgcgccgggtggcggtaacggtggccgtcct tcttcctcttac-3′ in accordance with the method described in Huemmerich et al.; Biochemistry. 43(42):13604-12, (2004) to give a 16mer and cloned into the expression vector pET21a (Novagen). The expression was carried out in the strain E. coli BLR [DE3] (Novagen), The expressed protein U16 with the sequence SEQ ID NO: 1 comprises N-terminally a T7 tag (amino acids No. 1-15) and then beyond amino acid No. 16 the actual resilin protein.

The cultivation and protein synthesis was carried out at 37° C., pO₂>20% and pH=6.8 in the fed-batch method.

### Medium

| | | |
|---|---|---|
| 8 | liters | Water |
| 230 | g | Yeast extract |
| 150 | g | Bacto-Tryptone |
| 30 | g | Glycerol (99%) |
| 20 | g | Potassium dihydrogenphosphate (KH₂PO₄) |
| 50 | g | Ammonium sulfate ((NH₄)₂SO₄) |
| 10.5 | g | Magnesium sulfate heptahydrate (MgSO₄ * 7H₂O) |
| 1.0 | g | Calcium chloride dihydrate (CaCl₂ * 2H₂O) |
| 100 | ml | Trace salt solution |
| | | top up to 9.7 liters with water |
| | | adjust pH to 6.8 with 25% strength NaOH |
| 3 | ml | Tego KS 911 (Antifoam; Goldschmidt Produkte) |
| 1 | g | Ampicillin |

### Trace Salt Solution:

| | | |
|---|---|---|
| 5 | liters | Water |
| 200.00 | g | Citric acid monohydrate |
| 55.00 | g | ZnSO₄ * 7H₂O |
| 42.50 | g | (NH₄)₂Fe(SO₄)₂ * 6H₂O |
| 15.00 | g | MnSO₄ * H₂O |
| 4.00 | g | CuSO₄ * 5H₂O |
| 1.25 | g | CoSO₄ * 7H₂O |

### Feeding Solution

| | |
|---|---|
| 800 g | Water |
| 30 g | Citric acid monohydrate |
| 60 g | Sodium sulfate (Na₂SO₄) |
| 4.5 g | (NH₄)₂Fe(SO₄)₂ * 6H₂O |
| 3400 g | Glycerol 99.5% |

After using up the glycerol present in the base medium, a constant feed was started at a rate of 100 ml/h.

The protein synthesis was induced by adding 1 mM isopropyl β-D-thiogalactopyranoside after the bacteria culture reached an optical density OD₆₀₀=4D. At this point, the temperature of the culture was reduced to 30° C. The cells were harvested 10 h after induction.

Purification of the protein was carried out in accordance with the following protocol:
resuspension of the cell pellet in 5 ml per gram of moist mass of 20 mM MOPS (3-(N-morpholino)propanesulfonic acid) pH 7.0
disruption of the cells in a high-pressure homogenizer
centrifugation for 30 min, 5000×g
dissolution of the pellet by adding 1.3 g of guanidinium hydrochloride (GdmHCl) per gram of pellet (end concentration 6 M GuaHCl)
dialysis against 20 mM citric acid pH 2.65
centrifugation for 45 min, 10 000×g
adjustment of the pH of the supernatant to 7.0 by adding 25% NaOH.
centrifugation 45 min, 10 000×g
precipitation of the protein from the supernatant by adding one volume of 4 M ammonium sulfate
centrifugation for 45 min, 10 000×g
the protein purified in this way can be stored as ammonium sulfate precipitate at −20° C.
for the use of the protein, the ammonium sulfate precipitate is taken up in GuaHCl, dialyzed against citric acid and neutralized.
the protein content in solution was determined photometrically (E_{(280; 1 mg/ml)}=0.93)
the purity of the protein was investigated by SDS-PAGE and by means of fluorescence spectroscopy (FIG. 1+2).

From a 10 l fermentation, 700 mg of clean U₁₆ protein with a purity of >95% were obtained.

Dermocosmetic preparations according to the invention comprising the resilin proteins prepared according to Example 1 are described below.

### Example 3

### Use of Resilin Protein in an Emulsion for Daycare—O/W Type

### Al 1%:

| | % | Ingredient (INCI) |
|---|---|---|
| A | 1.7 | Ceteareth-6, Stearyl Alcohol |
| | 0.7 | Ceteareth-25 |
| | 2.0 | Diethylamino Hydroxybenzoyl Hexyl Benzoate |
| | 2.0 | PEG-14 Dimethicone |
| | 3.6 | Cetearyl Alcohol |
| | 6.0 | Ethylhexyl Methoxycinnamate |
| | 2.0 | Dibutyl Adipate |
| B | 5.0 | Glycerin |
| | 0.2 | Disodium EDTA |
| | 1.0 | Panthenol |
| | q.s. | Preservative |
| | 67.8 | Aqua dem. |
| C | 4.0 | Caprylic/Capric Triglyceride, Sodium Acrylates Copolymer |
| D | 0.2 | Sodium Ascorbyl Phosphate |
| | 1.0 | Tocopheryl Acetate |
| | 0.2 | Bisabolol |
| | 1.0 | Caprylic/Capric Triglyceride, Sodium Ascorbate, Tocopherol, |
| | | Retinol |
| E | q.s. | Sodium Hydroxide |

### Al 5%:

| | % | Ingredient (INCI) |
|---|---|---|
| A | 1.7 | Ceteareth-6, Stearyl Alcohol |
| | 0.7 | Ceteareth-25 |
| | 2.0 | Diethylamino Hydroxybenzoyl Hexyl Benzoate |
| | 2.0 | PEG-14 Dimethicone |
| | 3.6 | Cetearyl Alcohol |
| | 6.0 | Ethylhexyl Methoxycinnamate |
| | 2.0 | Dibutyl Adipate |
| B | 5.0 | Glycerin |
| | 0.2 | Disodium EDTA |
| | 1.0 | Panthenol |
| | 5.0 | Resilin protein |
| | q.s. | Preservative |
| | 63.8 | Aqua dem. |
| C | 4.0 | Caprylic/Capric Triglyceride, Sodium Acrylates Copolymer |
| D | 0.2 | Sodium Ascorbyl Phosphate |
| | 1.0 | Tocopheryl Acetate |
| | 0.2 | Bisabolol |
| | 1.0 | Caprylic/Capric Triglyceride, Sodium Ascorbate, Tocopherol, |
| | | Retinol |
| E | q.s. | Sodium Hydroxide |

Preparation: Heat phases A and B separately from one another to about 80° C. Stir phase B into phase A and homogenize. Stir phase C into the combined phases A and B and homogenize again. Coot with stirring to about 40° C., add phase D, adjust the pH to about 6.5 with phase E, homogenize and cool to room temperature with stirring.

Note: The formulation is prepared without protective gas. Bottling must take place in oxygen-impermeable packagings, e.g. aluminum tubes.

### Example 4

### Use of Resilin Protein in a Protective Daycream—O/w Type

### Al 1%:

| | % | Ingredient (INCI) |
|---|---|---|
| A | 1.7 | Ceteareth-6, Stearyl Alcohol |
| | 0.7 | Ceteareth-25 |
| | 2.0 | Diethylamino Hydroxybenzoyl Hexyl Benzoate |
| | 2.0 | PEG-14 Dimethicone |
| | 3.6 | Cetearyl Alcohol |
| | 6.0 | Ethylhexyl Methoxycinnamate |
| | 2.0 | Dibutyl Adipate |
| B | 5.0 | Glycerin |
| | 0.2 | Disodium EDTA |
| | 1.0 | Panthenol |
| | 1.0 | Resilin protein |
| | q.s. | Preservative |
| | 68.6 | Aqua dem. |
| C | 4.0 | Caprylic/Capric Triglyceride, Sodium Acrylates Copolymer |
| D | 1.0 | Sodium Ascorbyl Phosphate |
| | 1.0 | Tocopheryl Acetate |
| | 0.2 | Bisabolol |
| E | q.s. | Sodium Hydroxide |

### Al 5%:

| | % | Ingredient (INCI) |
|---|---|---|
| A | 1.7 | Ceteareth-6, Stearyl Alcohol |
| | 0.7 | Ceteareth-25 |
| | 2.0 | Diethylamino Hydroxybenzoyl Hexyl Benzoate |
| | 2.0 | PEG-14 Dimethicone |
| | 3.6 | Cetearyl Alcohol |
| | 6.0 | Ethylhexyl Methoxycinnamate |
| | 2.0 | Dibutyl Adipate |
| B | 5.0 | Glycerin |
| | 0.2 | Disodium EDTA |
| | 1.0 | Panthenol |
| | 5.0 | Resilin protein |
| | q.s. | Preservative |
| | 64.6 | Aqua dem. |
| C | 4.0 | Caprylic/Capric Triglyceride, Sodium Acrylates Copolymer |
| D | 1.0 | Sodium Ascorbyl Phosphate |
| | 1.0 | Tocopheryl Acetate |
| | 0.2 | Bisabolol |
| E | q.s. | Sodium Hydroxide |

Preparation: Heat phases A and B separately from one another to about 80° C. Stir phase B into phase A and homogenize. Incorporate phase C into the combined phases A and B and homogenize. Cool to about 40° C. with stirring. Add phase D, adjust the pH to about 6.5 with phase E and homogenize. Cool to room temperature with stirring.

### Example 5

### Use of Resilin Protein in a Face Cleansing Lotion—O/W Type

### Al 1%:

| | % | Ingredient (INCI) |
|---|---|---|
| A | 10.0 | Cetearyl Ethylhexanoate |
| | 10.0 | Caprylic/Capric Triglyceride |
| | 1.5 | Cyclopentasiloxane, Cyclohexasilosane |
| | 2.0 | PEG-40 Hydrogenated Castor Oil |
| B | 3.5 | Caprylic/Capric Triglyceride, Sodium Acrylates Copolymer |
| C | 1.0 | Tocopheryl Acetate |
| | 0.2 | Bisabolol |
| | q.s. | Preservative |
| | q.s. | Perfume Oil |
| D | 3.0 | Polyquatemium-44 |
| | 0.5 | Cocotrimonium Methosulfate |
| | 0.5 | Ceteareth-25 |
| | 2.0 | Panthenol, Propylene Glycol |
| | 4.0 | Propylene Glycol |
| | 0.1 | Disodium EDTA |
| | 1.0 | Resilin protein |
| | 60.7 | Aqua dem. |

### Al 5%:

| | % | Ingredient (INCI) |
|---|---|---|
| A | 10.0 | Cetearyl Ethylhexanoate |
| | 10.0 | Caprylic/Capric Triglyceride |
| | 1.5 | Cyclopentasiloxane, Cyclohexasilosane |
| | 2.0 | PEG-40 Hydrogenated Castor Oil |
| B | 3.5 | Caprylic/Capric Triglyceride, Sodium Acrylates Copolymer |
| C | 1.0 | Tocopheryl Acetate |
| | 0.2 | Bisabolol |
| | q.s. | Preservative |
| | q.s. | Perfume Oil |
| D | 3.0 | Polyquaternium-44 |
| | 0.5 | Cocotrimonium Methosulfate |
| | 0.5 | Ceteareth-25 |
| | 2.0 | Panthenol, Propylene Glycol |
| | 4.0 | Propylene Glycol |
| | 0.1 | Disodium EDTA |
| | 5.0 | Resilin protein |
| | 56.7 | Aqua dem. |

Preparation: Dissolve phase A. Stir phase B into phase A, incorporate phase C into the combined phases A and B. Dissolve phase D, stir into the combined phases A, B and C and homogenize. After-stir for 15 min.

### Example 6

### Use of Resilin Protein in a Daily Care Body Spray

### Al 1%:

| | % | Ingredient (INCI) |
|---|---|---|
| A | 3.0 | Ethylhexyl Methoxycinnamate |
| | 2.0 | Diethylamino Hydroxybenzoyl Hexyl Benzoate |
| | 1.0 | Polyquaternium-44 |
| | 3.0 | Propylene Glycol |
| | 2.0 | Panthenol, Propylene Glycol |
| | 1.0 | Cyclopentasiloxane, Cyclohexasiloxane |
| | 10.0 | Octyldodecanol |
| | 0.5 | PVP |
| | 10.0 | Caprylic/Capric Triglyceride |
| | 3.0 | C12-15 Alkyl Benzoate |
| | 3.0 | Glycerin |
| | 1.0 | Tocopheryl Acetate |
| | 0.3 | Bisabolol |
| | 1.0 | Resilin protein |
| | 59.2 | Alcohol |

Al 5%:

| | % | Ingredient (INCI) |
|---|---|---|
| A | 3.0 | Ethylhexyl Methoxycinnamate |
| | 2.0 | Diethylamino Hydroxybenzoyl Hexyl Benzoate |
| | 1.0 | Polyquaternium-44 |
| | 3.0 | Propylene Glycol |
| | 2.0 | Panthenol, Propylene Glycol |
| | 1.0 | Cyclopentasiloxane, Cyclohexasiloxane |
| | 10.0 | Octyldodecanol |
| | 0.5 | PVP |
| | 10.0 | Caprylic/Capric Triglyceride |
| | 3.0 | C12-15 Alkyl Benzoate |
| | 3.0 | Glycerin |
| | 1.0 | Tocopheryl Acetate |
| | 0.3 | Bisabolol |
| | 5.0 | Resilin protein |
| | 55.2 | Alcohol |

Preparation: Weigh in the components of phase A and dissolve to give a clear solution.

### Example 7

### Use of Resilin Protein in a Skincare Gel

### Al 1%:

| | % | Ingredient (INCI) |
|---|---|---|
| A | 3.6 | PEG-40 Hydrogenated Castor Oil |
| | 15.0 | Alcohol |
| | 0.1 | Bisabolol |
| | 0.5 | Tocopheryl Acetate |
| | q.s. | Perfume Oil |
| B | 3.0 | Panthenol |
| | 0.6 | Carbomer |
| | 1.0 | Resilin protein |
| | 75.4 | Aqua dem. |
| C | 0.8 | Triethanolamine |

### Al 5%:

| | % | Ingredient (INCI) |
|---|---|---|
| A | 3.6 | PEG-40 Hydrogenated Castor Oil |
| | 15.0 | Alcohol |
| | 0.1 | Bisabolol |
| | 0.5 | Tocopheryl Acetate |
| | q.s. | Perfume Oil |
| B | 3.0 | Panthenol |
| | 0.6 | Carbomer |
| | 5.0 | Resilin protein |
| | 71.4 | Aqua dem. |
| C | 0.8 | Triethanolamine |

Preparation: Dissolve phase A to give a clear solution. Allow phase B to swell and neutralize with phase C. Stir phase A into the homogenized phase B and homogenize.

### Example 8

### Use of Resilin Protein in an Aftershave Lotion

### Al 1%:

| | % | Ingredient (INCI) |
|---|---|---|
| A | 10.0 | Cetearyl Ethylhexanoate |
| | 5.0 | Tocopheryl Acetate |
| | 1.0 | Bisabolol |
| | 0.1 | Perfume Oil |
| | 0.3 | Acrylates/C10-30 Alkyl Acrylate |
| | | Crosspolymer |
| B | 15.0 | Alcohol |
| | 1.0 | Panthenol |
| | 3.0 | Glycerin |
| | 1.0 | Resilin protein |
| | 0.1 | Triethanolamine |
| | 63.5 | Aqua dem. |

### Al 5%:

| | % | Ingredient (INCI) |
|---|---|---|
| A | 10.0 | Cetearyl Ethylhexanoate |
| | 5.0 | Tocopheryl Acetate |
| | 1.0 | Bisabolol |
| | 0.1 | Perfume Oil |
| | 0.3 | Acrylates/C10-30 Alkyl Acrylate |
| | | Crosspolymer |
| B | 15.0 | Alcohol |
| | 1.0 | Panthenol |
| | 3.0 | Glycerin |
| | 5.0 | Resilin protein |
| | 0.1 | Triethanolamine |
| | 59.5 | Aqua dem. |

Preparation: Mix the components of phase A. Dissolve phase B, incorporate into phase A and homogenize.

### Example 9

### Use of Resilin Protein in an Aftersun Lotion

### Al 1%:

| | % | Ingredient (INCI) |
|---|---|---|
| A | 0.4 | Acrylates/C10-30 Alkyl Acrylate |
| | | Crosspolymer |
| | 15.0 | Cetearyl Ethylhexanoate |
| | 0.2 | Bisabolol |
| | 1.0 | Tocopheryl Acetate |
| | q.s. | Perfume Oil |
| B | 1.0 | Panthenol |
| | 15.0 | Alcohol |
| | 3.0 | Glycerin |
| | 1.0 | Resilin protein |
| | 63.2 | Aqua dem. |
| C | 0.2 | Triethanolamine |

### Al 5%:

| | % | Ingredient (INCI) |
|---|---|---|
| A | 0.4 | Acrylates/C10-30 Alkyl Acrylate |
| | | Crosspolymer |
| | 15.0 | Cetearyl Ethylhexanoate |
| | 0.2 | Bisabolol |
| | 1.0 | Tocopheryl Acetate |
| | q.s. | Perfume Oil |
| B | 1.0 | Panthenol |
| | 15.0 | Alcohol |
| | 3.0 | Glycerin |
| | 5.0 | Resilin protein |
| | 59.2 | Aqua dem. |
| C | 0.2 | Triethanolamine |

Preparation: Mix the components of phase A. Stir phase B into phase A with homogenization. Neutralize with phase C and homogenize again.

### Example 10

### Use of Resilin Protein in a Sunscreen Lotion

### Al 1%:

| | % | Ingredient (INCI) |
|---|---|---|
| A | 4.5 | Ethylhexyl Methoxycinnamate |
| | 2.0 | Diethylamino Hydroxybenzoyl Hexyl Benzoate |
| | 3.0 | Octocrylene |
| | 2.5 | Di-C12-13 Alkyl Malate |
| | 0.5 | Tocopheryl Acetate |
| | 4.0 | Polyglyceryl-3 Methyl Glucose Distearate |
| B | 3.5 | Cetearyl Isononanoate |
| | 1.0 | VP/Eicosene Copolymer |
| | 5.0 | Isohexadecane |
| | 2.5 | Di-C12-13 Alkyl Malate |
| | 3.0 | Titanium Dioxide, Trimethoxycaprylylsilane |
| C | 5.0 | Glycerin |
| | 1.0 | Sodium Cetearyl Sulfate |
| | 0.5 | Xanthan Gum |
| D | 1.0 | Resilin protein |
| | 59.7 | Aqua dem. |
| | 1.0 | Phenoxyethanol, Methylparaben, Ethylparaben, |
| | | Butylparaben, Propylparaben, Isobutylparaben |
| | 0.3 | Bisabolol |

### Al 5%:

| | % | Ingredient (INCI) |
|---|---|---|
| A | 4.5 | Ethylhexyl Methoxycinnamate |
| | 2.0 | Diethylamino Hydroxybenzoyl Hexyl Benzoate |
| | 3.0 | Octocrylene |
| | 2.5 | Di-C12-13 Alkyl Malate |
| | 0.5 | Tocopheryl Acetate |
| | 4.0 | Polyglyceryl-3 Methyl Glucose Distearate |
| B | 3.5 | Cetearyl Isononanoate |
| | 1.0 | VP/Eicosene Copolymer |
| | 5.0 | Isohexadecane |
| | 2.5 | Di-C12-13 Alkyl Malate |
| | 3.0 | Titanium Dioxide, Trimethoxycaprylylsilane |
| C | 5.0 | Glycerin |
| | 1.0 | Sodium Cetearyl Sulfate |
| | 0.5 | Xanthan Gum |
| D | 5.0 | Resilin protein |
| | 55.7 | Aqua dem. |
| | 1.0 | Phenoxyethanol, Methylparaben, Ethylparaben, |
| | | Butylparaben, Propylparaben, Isobutylparaben |
| | 0.3 | Bisabolol |

Preparation: Heat the components of phase A and B separately from one another to about 80° C. Stir phase B into phase A and homogenize. Heat phase C to about 80° C. and stir into the combined phases A and B with homogenization. Cool to about 40° C. with stirring, add phase D and homogenize again.

### Example 11

### Use of Resilin Protein in a Sunscreen Lotion—O/W Type

### Al 1%:

| | % | Ingredient (INCI) |
|---|---|---|
| A | 2.0 | Ceteareth-6, Stearyl Alcohol |
| | 2.0 | Ceteareth-25 |
| | 3.0 | Tribehenin |
| | 2.0 | Cetearyl Alcohol |
| | 2.0 | Cetearyl Ethylhexanoate |
| | 5.0 | Ethylhexyl Methoxycinnamate |
| | 1.0 | Ethylhexyl Triazone |
| | 1.0 | VP/Eicosene Copolymer |
| | 7.0 | Isopropyl Myristate |
| B | 5.0 | Zinc Oxide, Triethoxycaprylylsilane |
| C | 0.2 | Xanthan Gum |
| | 0.5 | Hydroxyethyl Acrylate/Sodium Acryloyldimethyl |
| | | Taurate Copolymer, Squalane, Polysorbate 60 |
| | 0.2 | Disodium EDTA |
| | 5.0 | Propylene Glycol |
| | 0.5 | Panthenol |
| D | 1.0 | Resilin protein |
| | 60.9 | Aqua dem. |
| | 0.5 | Phenoxyethanol, Methylparaben, Butylparaben, |
| | | Ethylparaben, Propylparaben, Isopropylparaben |
| | 1.0 | Tocopheryl Acetate |
| | 0.2 | Bisabolol |

### Al 5%:

| | % | Ingredient (INCI) |
|---|---|---|
| A | 2.0 | Ceteareth-6, Stearyl Alcohol |
| | 2.0 | Ceteareth-25 |
| | 3.0 | Tribehenin |
| | 2.0 | Cetearyl Alcohol |
| | 2.0 | Cetearyl Ethylhexanoate |
| | 5.0 | Ethylhexyl Methoxycinnamate |
| | 1.0 | Ethylhexyl Triazone |
| | 1.0 | VP/Eicosene Copolymer |
| | 7.0 | Isopropyl Myristate |
| B | 5.0 | Zinc Oxide, Triethoxycaprylylsilane |
| C | 0.2 | Xanthan Gum |
| | 0.5 | Hydroxyethyl Acrylate/Sodium Acryloyldimethyl |
| | | Taurate Copolymer, Squalane, Polysorbate 60 |
| | 0.2 | Disodium EDTA |
| | 5.0 | Propylene Glycol |
| | 0.5 | Panthenol |
| D | 5.0 | Resilin protein |
| | 56.9 | Aqua dem. |
| | 0.5 | Phenoxyethanol, Methylparaben, Butylparaben, |
| | | Ethylparaben, Propylparaben, Isopropylparaben |
| | 1.0 | Tocopheryl Acetate |
| | 0.2 | Bisabolol |

Preparation: Heat phase A to about 80° C., stir in phase B and homogenize for 3 min. Heat phase C likewise to 80° C. and stir into the combined phases A and B with homogenization. Cool to about 40° C., stir in phase D and homogenize again.

### Example 12

### Use of Resilin Protein in a Sunscreen Lotion—O/W Type

### Al 1%:

| | % | Ingredient (INCI) |
|---|---|---|
| A | 3.5 | Ceteareth-6, Stearyl Alcohol |
| | 1.5 | Ceteareth-25 |
| | 7.5 | Ethylhexyl Methoxycinnamate |
| | 2.0 | Diethylamino Hydroxybenzoyl Hexyl Benzoate |
| | 2.0 | Cyclopentasiloxane, Cyclohexasiloxane |
| | 0.5 | Bees Wax |
| | 3.0 | Cetearyl Alcohol |
| | 10.0 | Caprylic/Capric Triglyceride |
| B | 5.0 | Titanium Dioxide, Silica, Methicone, Alumina |
| C | 3.0 | Glycerin |
| | 0.2 | Disodium EDTA |
| | 0.3 | Xanthan Gum |
| | 1.0 | Decyl Glucoside |
| | 2.0 | Panthenol, Propylene Glycol |
| | 1.0 | Resilin protein |
| | 56.3 | Aqua dem. |
| D | 1.0 | Tocopheryl Acetate |
| | 0.2 | Bisabolol |
| | q.s. | Perfume Oil |
| | q.s. | Preservative |

### Al 5%:

| | % | Ingredient (INCI) |
|---|---|---|
| A | 3.5 | Ceteareth-6, Stearyl Alcohol |
| | 1.5 | Ceteareth-25 |
| | 7.5 | Ethylhexyl Methoxycinnamate |
| | 2.0 | Diethylamino Hydroxybenzoyl Hexyl Benzoate |
| | 2.0 | Cyclopentasiloxane, Cyclohexasiloxane |
| | 0.5 | Bees Wax |
| | 3.0 | Cetearyl Alcohol |
| | 10.0 | Caprylic/Capric Triglyceride |
| B | 5.0 | Titanium Dioxide, Silica, Methicone, Alumina |
| C | 3.0 | Glycerin |
| | 0.2 | Disodium EDTA |
| | 0.3 | Xanthan Gum |
| | 1.0 | Decyl Glucoside |
| | 2.0 | Panthenol, Propylene Glycol |
| | 5.0 | Resilin protein |
| | 52.3 | Aqua dem. |
| D | 1.0 | Tocopheryl Acetate |
| | 0.2 | Bisabolol |
| | q.s. | Perfume Oil |
| | q.s. | Preservative |

Preparation: Heat phase A to about 80° C., stir in phase B and homogenize for 3 min. Likewise heat phase C to 80° C. and stir into the combined phases A an B with homogenization. Cool to about 40° C., stir in phase D and homogenize again.

### Example 13

### Use of Resilin Protein in a Foot Balm

### Al 1%:

| | % | Ingredient (INCI) |
|---|---|---|
| A | 2.0 | Ceteareth-6, Stearyl Alcohol |
| | 2.0 | Ceteareth-25 |
| | 5.0 | Cetearyl Ethylhexanoate |
| | 4.0 | Cetyl Alcohol |
| | 4.0 | Glyceryl Stearate |
| | 5.0 | Mineral Oil |
| | 0.2 | Menthol |
| | 0.5 | Camphor |
| B | 69.3 | Aqua dem. |
| | 1.0 | Resilin protein |
| | q.s. | Preservative |
| C | 1.0 | Bisabolol |
| | 1.0 | Tocopheryl Acetate |
| D | 5.0 | Witch Hazel Extract |

### Al 5%:

| | % | Ingredient (INCI) |
|---|---|---|
| A | 2.0 | Ceteareth-6, Stearyl Alcohol |
| | 2.0 | Ceteareth-25 |
| | 5.0 | Cetearyl Ethylhexanoate |
| | 4.0 | Cetyl Alcohol |
| | 4.0 | Glyceryl Stearate |
| | 5.0 | Mineral Oil |
| | 0.2 | Menthol |
| | 0.5 | Camphor |
| B | 65.3 | Aqua dem. |
| | 5.0 | Resilin protein |
| | q.s. | Preservative |
| C | 1.0 | Bisabolol |
| | 1.0 | Tocopheryl Acetate |
| D | 5.0 | Witch Hazel Extract |

Preparation: Heat the components of phases A and B separately from one another to about 80° C. Stir phase B into phase A with homogenization. Cool to about 40° C. with stirring, add phases C and D and briefly after-homogenize. Cool to room temperature with stirring.

### Example 14

### Use of Resilin Protein in a W/O Emulsion with Bisabolol

### Al 1%:

| | % | Ingredient (INCI) |
|---|---|---|
| A | 6.0 | PEG-7 Hydrogenated Castor Oil |
| | 8.0 | Cetearyl Ethylhexanoate |
| | 5.0 | Isopropyl Myristate |
| | 15.0 | Mineral Oil |
| | 0.3 | Magnesium Stearate |
| | 0.3 | Aluminum Stearate |
| | 2.0 | PEG-45/Dodecyl Glycol Copolymer |
| B | 5.0 | Glycerin |
| | 0.7 | Magnesium Sulfate |
| | 1.0 | Resilin protein |
| | 55.6 | Aqua dem. |
| C | 0.5 | Tocopheryl Acetate |
| | 0.6 | Bisabolol |

### Al 5%:

| | % | Ingredient (INCI) |
|---|---|---|
| A | 6.0 | PEG-7 Hydrogenated Castor Oil |
| | 8.0 | Cetearyl Ethylhexanoate |
| | 5.0 | Isopropyl Myristate |
| | 15.0 | Mineral Oil |
| | 0.3 | Magnesium Stearate |
| | 0.3 | Aluminum Stearate |
| | 2.0 | PEG-45/Dodecyl Glycol Copolymer |
| B | 5.0 | Glycerin |
| | 0.7 | Magnesium Sulfate |
| | 51.6 | Aqua dem. |
| C | 5.0 | Resilin protein |
| | 0.5 | Tocopheryl Acetate |

Preparation: Heat phases A and B separately from one another to about 85° C. Stir phase B into phase A and homogenize. Cool to about 40° C. with stirring, add phase C and briefly homogenize again. Cool to room temperature with stirring.

### Example 15

### Foam Conditioner with Setting Agent

### Al 1%

| | % | Ingredient (INCI) |
|---|---|---|
| A | 10.0 | PVP/VA Copolymer |
| | 0.2 | Hydroxyethyl Cetyldimonium Phosphate |
| | 0.2 | Ceteareth-25 |
| | 0.5 | Dimethicone Copolyol |
| | q.s. | Perfume Oil |
| | 10.0 | Alcohol |
| | 1.0 | Resilin protein |
| | 68.1 | Aqua dem. |
| | 10.0 | Propane/Butane |

### Al 5%

| | % | Ingredient (INCI) |
|---|---|---|
| A | 10.0 | PVP/VA Copolymer |
| | 0.2 | Hydroxyethyl Cetyldimonium Phosphate |
| | 0.2 | Ceteareth-25 |
| | 0.5 | Dimethicone Copolyol |
| | q.s. | Perfume Oil |
| | 10.0 | Alcohol |
| | 5.0 | Resilin protein |
| | 64.1 | Aqua dem. |
| | 10.0 | Propane/Butane |

Preparation: Weigh the components of phase A together, stir until everything has dissolved and bottle.

### Example 16

### Foam Conditioner

### Al 1%

| | % | Ingredient (INCI) |
|---|---|---|
| A | 1.0 | Polyquaternium-4 |
| | 0.5 | Hydroxyethyl Cetyldimonium Phosphate |
| | 1.0 | Resilin protein |
| | q.s. | Perfume Oil |
| | q.s. | Preservative |
| | 91.5 | Aqua dem. |
| | 6.0 | Propane/Butane |

### Al 5%

| | % | Ingredient (INCI) |
|---|---|---|
| A | 1.0 | Polyquaternium-4 |
| | 0.5 | Hydroxyethyl Cetyldimonium Phosphate |
| | 5.0 | Resilin protein |
| | q.s. | Perfume Oil |
| | q.s. | Preservative |
| | 87.5 | Aqua dem. |
| | 6.0 | Propane/Butane |

Preparation: Weigh the components of phase A together, stir until everything has dissolved to give a clear solution and bottle.

### Example 17

### Foam Conditioner

### Al 1%

| | % | Ingredient (INCI) |
|---|---|---|
| A | 1.0 | Polyquaternium-11 |
| | 0.5 | Hydroxyethyl Cetyldimonium Phosphate |
| | 1.0 | Resilin protein |
| | q.s. | Perfume Oil |
| | q.s. | Preservative |
| | 91.5 | Aqua dem. |
| | 6.0 | Propane/Butane |

### Al 5%

| | % | Ingredient (INCI) |
|---|---|---|
| A | 1.0 | Polyquaternium-11 |
| | 0.5 | Hydroxyethyl Cetyldimonium Phosphate |
| | 5.0 | Resilin protein |
| | q.s. | Perfume Oil |
| | q.s. | Preservative |
| | 87.5 | Aqua dem. |
| | 6.0 | Propane/Butane |

Preparation: Weigh the components of phase A together, stir until everything has dissolved to give a clear solution and bottle.

### Example 18

### Styling Foam

### Al 1%

| | % | Ingredient (INCI) |
|---|---|---|
| A | 0.5 | Laureth-4 |
| | q.s. | Perfume Oil |
| B | 77.3 | Aqua dem. |
| | 10.0 | Polyquaternium-28 |
| | 1.0 | Resilin protein |
| | 0.5 | Dimethicone Copolyol |
| | 0.2 | Ceteareth-25 |
| | 0.2 | Panthenol |
| | 0.1 | PEG-25 PABA |
| | 0.2 | Hydroxyethylcellulose |
| C | 10.0 | HFC 152 A |

### Al 5%

| | % | Ingredient (INCI) |
|---|---|---|
| A | 0.5 | Laureth-4 |
| | q.s. | Perfume Oil |
| B | 73.3 | Aqua dem. |
| | 10.0 | Polyquaternium-28 |
| | 5.0 | Resilin protein |
| | 0.5 | Dimethicone Copolyol |
| | 0.2 | Ceteareth-25 |
| | 0.2 | Panthenol |
| | 0.1 | PEG-25 PABA |
| | 0.2 | Hydroxyethylcellulose |
| C | 10.0 | HFC 152 A |

Preparation: Mix the components of phase A. Add the components of phase B one after the other and dissolve. Bottle with phase C.

### Example 19

### Styling Foam

### Al 1%

| | % | Ingredient (INCI) |
|---|---|---|
| A | 2.0 | Cocotrimonium Methosulfate |
| | q.s. | Perfume Oil |
| B | 78.5 | Aqua dem. |
| | 6.7 | Acrylates Copolymer |
| | 0.6 | AMP |
| | 1.0 | Resilin protein |
| | 0.5 | Dimethicone Copolyol |
| | 0.2 | Ceteareth-25 |
| | 0.2 | Panthenol |
| | 0.1 | PEG-25 PABA |
| | 0.2 | Hydroxyethylcellulose |
| C | 10.0 | HFC 152 A |

### Al 5%

| | % | Ingredient (INCI) |
|---|---|---|
| A | 2.0 | Cocotrimonium Methosulfate |
| | q.s. | Perfume Oil |
| B | 74.5 | Aqua dem. |
| | 6.7 | Acrylates Copolymer |
| | 0.6 | AMP |
| | 5.0 | Resilin protein |
| | 0.5 | Dimethicone Copolyol |
| | 0.2 | Ceteareth-25 |
| | 0.2 | Panthenol |
| | 0.1 | PEG-25 PABA |
| | 0.2 | Hydroxyethylcellulose |
| C | 10.0 | HFC 152 A |

Preparation: Mix the components of phase A. Add the components of phase B one after the other and dissolve. Bottle with phase C.

### Example 20

### Styling Foam

### Al 1%

| | % | Ingredient (INCI) |
|---|---|---|
| A | 2.0 | Cocotrimonium Methosulfate |
| | q.s. | Perfume Oil |
| B | 7.70 | Polyquaternium-44 |
| | 1.0 | Resilin protein |
| | q.s. | Preservative |
| | 79.3 | Aqua dem. |
| C | 10.0 | Propane/Butane |

### Al 5%

| | % | Ingredient (INCI) |
|---|---|---|
| A | 2.0 | Cocotrimonium Methosulfate |
| | q.s. | Perfume Oil |
| B | 7.70 | Polyquaternium-44 |
| | 5.0 | Resilin protein |
| | q.s. | Preservative |
| | 75.3 | Aqua dem. |
| C | 10.0 | Propane/Butane |

Preparation: Mix the components of phase A. Dissolve the components of phase B to give a clear solution, then stir phase B into phase A. Adjust the pH to 6-7, bottle with phase C.

### Example 21

### Styling Foam

### Al 1%

| | % | Ingredient (INCI) |
|---|---|---|
| A | 2.00 | Cocotrimonium Methosulfate |
| | q.s. | Perfume Oil |
| B | 72.32 | Aqua dem. |
| | 2.00 | VP/Acrylates/Lauryl Methacrylate Copolymer |
| | 0.53 | AMP |
| | 1.00 | Resilin protein |
| | 0.20 | Ceteareth-25 |
| | 0.50 | Panthenol |
| | 0.05 | Benzophenone-4 |
| | 0.20 | Amodimethicone, Cetrimonium Chloride, Trideceth-12 |
| | 15.00 | Alcohol |
| C | 0.20 | Hydroxyethylcellulose |
| D | 6.00 | Propane/Butane |

### Al 5%

| | % | Ingredient (INCI) |
|---|---|---|
| A | 2.00 | Cocotrimonium Methosulfate |
| | q.s. | Perfume Oil |
| B | 68.32 | Aqua dem. |
| | 2.00 | VP/Acrylates/Lauryl Methacrylate Copolymer |
| | 0.53 | AMP |
| | 5.00 | Resilin protein |
| | 0.20 | Ceteareth-25 |
| | 0.50 | Panthenol |
| | 0.05 | Benzophenone-4 |
| | 0.20 | Amodimethicone, Cetrimonium Chloride, Trideceth-12 |
| | 15.00 | Alcohol |
| C | 0.20 | Hydroxyethylcellulose |
| D | 6.00 | Propane/Butane |

Preparation: Mix the components of phase A. Add the components of phase B one after the other and dissolve. Dissolve phase C in the mixture with A and B, then adjust the pH to 6-7. Bottle with phase D.

### Example 22

### Styling Foam

### Al 1%

| | % | Ingredient (INCI) |
|---|---|---|
| A | 2.00 | Cetrimonium Chloride |
| | q.s. | Perfume Oil |
| B | 67.85 | Aqua dem. |
| | 7.00 | Polyquaternium-46 |
| | 1.00 | Resilin protein |
| | 0.20 | Ceteareth-25 |
| | 0.50 | Panthenol |
| | 0.05 | Benzophenone-4 |
| | 0.20 | Amodimethicone, Cetrimonium |
| | | Chloride, Trideceth-12 |
| | 15.00 | Alcohol |
| C | 0.20 | Hydroxyethylcellulose |
| D | 6.00 | Propane/Butane |

### Al 5%

| | % | Ingredient (INCI) |
|---|---|---|
| A | 2.00 | Cetrimonium Chloride |
| | q.s. | Perfume Oil |
| B | 63.85 | Aqua dem. |
| | 7.00 | Polyquaternium-46 |
| | 5.00 | Resilin protein |
| | 0.20 | Ceteareth-25 |
| | 0.50 | Panthenol |
| | 0.05 | Benzophenone-4 |
| | 0.20 | Amodimethicone, Cetrimonium |
| | | Chloride, Trideceth-12 |
| | 15.00 | Alcohol |
| C | 0.20 | Hydroxyethylcellulose |
| D | 6.00 | Propane/Butane |

Preparation: Mix the components of phase A. Add the components of phase B one after the other and dissolve. Dissolve phase C in the mixture with A and B, then adjust the pH to 6-7. Bottle with phase D.

### Example 23

### Styling Foam

### Al 1%

| | % | Ingredient (INCI) |
|---|---|---|
| A | q.s. | PEG-40 Hydrogenated Castor Oil |
| | q.s. | Perfume Oil |
| | 85.5 | Aqua dem. |
| B | 7.0 | Sodium Polystyrene Sulfonate |
| | 1.0 | Resilin protein |
| | 0.5 | Cetrimonium Bromide |
| | q.s. | Preservative |
| C | 6.0 | Propane/Butane |

### Styling Foam

### Al 5%

| | % | Ingredient (INCI) |
|---|---|---|
| A | q.s. | PEG-40 Hydrogenated Castor Oil |
| | q.s. | Perfume Oil |
| | 81.5 | Aqua dem. |
| B | 7.0 | Sodium Polystyrene Sulfonate |
| | 5.0 | Resilin protein |
| | 0.5 | Cetrimonium Bromide |
| | q.s. | Preservative |
| C | 6.0 | Propane/Butane |

Preparation: Solubilize phase A. Weigh phase B into phase A and dissolve to give a clear solution. Adjust the pH to 6-7, bottle with phase C.

### Example 24

### Styling Foam

### Al 1%

| | % | Ingredient (INCI) |
|---|---|---|
| A | q.s. | PEG-40 Hydrogenated Castor Oil |
| | q.s. | Perfume Oil |
| | 92.0 | Aqua dem. |
| B | 0.5 | Polyquaternium-10 |
| | 1.0 | Resilin protein |
| | 0.5 | Cetrimonium Bromide |
| | q.s. | Preservative |
| C | 6.0 | Propane/Butane |

### Al 5%

| | % | Ingredient (INCI) |
|---|---|---|
| A | q.s. | PEG-40 Hydrogenated Castor Oil |
| | q.s. | Perfume Oil |
| | 88.0 | Aqua dem. |
| B | 0.5 | Polyquaternium-10 |
| | 5.0 | Resilin protein |
| | 0.5 | Cetrimonium Bromide |
| | q.s. | Preservative |
| C | 6.0 | Propane/Butane |

Preparation: Solubilize phase A. Weigh phase B into phase A and dissolve to give a clear solution. Adjust the pH to 6-7, bottle with phase C.

### Example 25

### Styling Foam

### Al 1%

| | % | Ingredient (INCI) |
|---|---|---|
| A | q.s. | PEG-40 Hydrogenated Castor Oil |
| | q.s. | Perfume Oil |
| | 82.5 | Aqua dem. |
| B | 10.0 | Polyquaternium-16 |
| | 1.0 | Resilin protein |
| | 0.5 | Hydroxyethyl Cetyldimonium Phosphate |
| | q.s. | Preservative |
| C | 6.0 | Propane/Butane |

### Al 5%

| | % | Ingredient (INCI) |
|---|---|---|
| A | q.s. | PEG-40 Hydrogenated Castor Oil |
| | q.s. | Perfume Oil |
| | 78.5 | Aqua dem. |
| B | 10.0 | Polyquaternium-16 |
| | 5.0 | Resilin protein |
| | 0.5 | Hydroxyethyl Cetyldimonium Phosphate |
| | q.s. | Preservative |
| C | 6.0 | Propane/Butane |

Preparation: Solubilize phase A. Weigh phase B into phase A and dissolve to give a clear solution. Adjust the pH to 6-7, bottle with phase C.

### Example 26

### Styling Foam

### Al 1%

| | % | Ingredient (INCI) |
|---|---|---|
| A | 2.0 | Cocotrimonium Methosulfate |
| | q.s. | Perfume Oil |
| B | 84.0 | Aqua dem. |
| | 2.0 | Chitosan |
| | 1.0 | Resilin protein |
| | 0.5 | Dimethicone Copolyol |
| | 0.2 | Ceteareth-25 |
| | 0.2 | Panthenol |
| | 0.1 | PEG-25 PABA |
| C | 10.0 | HFC 152 A |

### Al 5%

| | % | Ingredient (INCI) |
|---|---|---|
| A | 2.0 | Cocotrimonium Methosulfate |
| | q.s. | Perfume Oil |
| B | 80.0 | Aqua dem. |
| | 2.0 | Chitosan |
| | 5.0 | Resilin protein |
| | 0.5 | Dimethicone Copolyol |
| | 0.2 | Ceteareth-25 |
| | 0.2 | Panthenol |
| | 0.1 | PEG-25 PABA |
| C | 10.0 | HFC 152 A |

Preparation: Mix the components of phase A. Add the components of phase B one after the other and dissolve. Bottle with phase C.

### Example 27

### Care Shampoo

### Al 1%

| | % | Ingredient (INCI) |
|---|---|---|
| A | 30.0 | Sodium Laureth Sulfate |
| | 6.0 | Sodium Cocoamphoacetate |
| | 6.0 | Cocamidopropyl Betaine |
| | 3.0 | Sodium Laureth Sulfate, Glycol Distearate, |
| | | Cocamide MEA, Laureth-10 |
| | 1.0 | Resilin protein |
| | 7.7 | Polyquaternium-44 |
| | 2.0 | Amodimethicone |
| | q.s. | Perfume Oil |
| | q.s. | Preservative |
| | 1.0 | Sodium Chloride |
| | 43.3 | Aqua dem. |
| B | q.s. | Citric Acid |

### Al 5%

| | % | Ingredient (INCI) |
|---|---|---|
| A | 30.0 | Sodium Laureth Sulfate |
| | 6.0 | Sodium Cocoamphoacetate |
| | 6.0 | Cocamidopropyl Betaine |
| | 3.0 | Sodium Laureth Sulfate, Glycol Distearate, |
| | | Cocamide MEA, Laureth-10 |
| | 5.0 | Resilin protein |
| | 7.7 | Polyquaternium-44 |
| | 2.0 | Amodimethicone |
| | q.s. | Perfume Oil |
| | q.s. | Preservative |
| | 1.0 | Sodium Chloride |
| | 39.3 | Aqua dem. |
| B | q.s. | Citric Acid |

Preparation: Mix the components of phase A and dissolve. Adjust the pH to 6-7 with citric acid.

### Example 28

### Shower Gel

### Al 1%

| | % | Ingredient (INCI) |
|---|---|---|
| A | 40.0 | Sodium Laureth Sulfate |
| | 5.0 | Decyl Glucoside |
| | 5.0 | Cocamidopropyl Betaine |
| | 1.0 | Resilin protein |
| | 1.0 | Panthenol |
| | q.s. | Perfume Oil |
| | q.s. | Preservative |
| | 2.0 | Sodium Chloride |
| | 46.0 | Aqua dem. |
| B | q.s. | Citric Acid |

### Al 5%

| | % | Ingredient (INCI) |
|---|---|---|
| A | 40.0 | Sodium Laureth Sulfate |
| | 5.0 | Decyl Glucoside |
| | 5.0 | Cocamidopropyl Betaine |
| | 5.0 | Resilin protein |
| | 1.0 | Panthenol |
| | q.s. | Perfume Oil |
| | q.s. | Preservative |
| | 2.0 | Sodium Chloride |
| | 42.0 | Aqua dem. |
| B | q.s. | Citric Acid |

Preparation: Mix the components of phase A and dissolve. Adjust the pH to 6-7 with citric acid.

### Example 29

### Shampoo

### Al 1%

| | % | Ingredient (INCI) |
|---|---|---|
| A | 40.0 | Sodium Laureth Sulfate |
| | 5.0 | Sodium C12-15 Pareth-15 Sulfonate |
| | 5.0 | Decyl Glucoside |
| | q.s. | Perfume Oil |
| | 0.1 | Phytantriol |
| | 44.6 | Aqua dem. |
| | 1.0 | Resilin protein |
| | 0.3 | Polyquaternium-10 |
| | 1.0 | Panthenol |
| | q.s. | Preservative |
| | 1.0 | Laureth-3 |
| | 2.0 | Sodium Chloride |

### Al 5%

| | % | Ingredient (INCI) |
|---|---|---|
| A | 40.0 | Sodium Laureth Sulfate |
| | 5.0 | Sodium C12-15 Pareth-15 Sulfonate |
| | 5.0 | Decyl Glucoside |
| | q.s. | Perfume Oil |
| | 0.1 | Phytantriol |
| | 40.6 | Aqua dem. |
| | 5.0 | Resilin protein |
| | 0.3 | Polyquaternium-10 |
| | 1.0 | Panthenol |
| | q.s. | Preservative |
| | 1.0 | Laureth-3 |
| | 2.0 | Sodium Chloride |

Preparation: Mix the components of phase A and dissolve. Adjust the pH to 6-7 with citric acid.

### Example 30

### Shampoo

### Al 1%

| | % | Ingredient (INCI) |
|---|---|---|
| A | 15.00 | Cocamidopropyl Betaine |
| | 10.00 | Disodium Cocoamphodiacetate |
| | 5.00 | Polysorbate 20 |
| | 5.00 | Decyl Glucoside |
| | q.s. | Perfume Oil |
| | q.s. | Preservative |
| | 1.00 | Resilin protein |
| | 0.15 | Guar Hydroxypropyltrimonium Chloride |
| | 2.00 | Laureth-3 |
| | 58.00 | Aqua dem. |
| | q.s. | Citric Acid |
| B | 3.00 | PEG-150 Distearate |

### Al 5%

| | % | Ingredient (INCI) |
|---|---|---|
| A | 15.00 | Cocamidopropyl Betaine |
| | 10.00 | Disodium Cocoamphodiacetate |
| | 5.00 | Polysorbate 20 |
| | 5.00 | Decyl Glucoside |
| | q.s. | Perfume Oil |
| | q.s. | Preservative |
| | 5.00 | Resilin protein |
| | 0.15 | Guar Hydroxypropyltrimonium Chloride |
| | 2.00 | Laureth-3 |
| | 54.00 | Aqua dem. |
| | q.s. | Citric Acid |
| B | 3.00 | PEG-150 Distearate |

Preparation: Weigh in the components of phase A and dissolve. Adjust the pH to 6-7, Add phase B and heat to about 50° C. Cool to room temperature with stirring.

### Example 31

### Moisturizing Bodycare Cream

### Al 1%

| | % | Ingredient (INCI) |
|---|---|---|
| A | 2.0 | Ceteareth-25 |
| | 2.0 | Ceteareth-6, Stearyl Alcohol |
| | 3.0 | Cetearyl Ethylhexanoate |
| | 1.0 | Dimethicone |
| | 4.0 | Cetearyl Alcohol |
| | 3.0 | Glyceryl Stearate SE |
| | 5.0 | Mineral Oil |
| | 4.0 | Simmondsia Chinensis (Jojoba) Seed Oil |
| | 3.0 | Mineral Oil, Lanolin Alcohol |
| B | 5.0 | Propylene Glycol |
| | 1.0 | Resilin protein |
| | 1.0 | Panthenol |
| | 0.5 | Magnesium Aluminum Silicate |
| | q.s. | Preservative |
| | 65.5 | Aqua dem. |
| C | q.s. | Perfume Oil |
| D | q.s. | Citric Acid |

### Al 5%

| | % | Ingredient (INCI) |
|---|---|---|
| A | 2.0 | Ceteareth-25 |
| | 2.0 | Ceteareth-6, Stearyl Alcohol |
| | 3.0 | Cetearyl Ethylhexanoate |
| | 1.0 | Dimethicone |
| | 4.0 | Cetearyl Alcohol |
| | 3.0 | Glyceryl Stearate SE |
| | 5.0 | Mineral Oil |
| | 4.0 | Simmondsia Chinensis (Jojoba) Seed Oil |
| | 3.0 | Mineral Oil, Lanolin Alcohol |
| B | 5.0 | Propylene Glycol |
| | 5.0 | Resilin protein |
| | 1.0 | Panthenol |
| | 0.5 | Magnesium Aluminum Silicate |
| | q.s | Preservative |
| | 61.5 | Aqua dem. |
| C | q.s. | Perfume Oil |
| D | q.s. | Citric Acid |

Preparation: Heat phases A and B separately to about 80° C. Briefly prehomogenize phase B, then stir phase B into phase A and homogenize again. Cool to about 40° C., add phase C and homogenize well again. Adjust the pH to 6-7 with citric acid.

### Example 32

### Moisturizing Bodycare Cream

### Al 1%

| | % | Ingredient (INCI) |
|---|---|---|
| A | 6.0 | PEG-7 Hydrogenated Castor Oil |
| | 10.0 | Cetearyl Ethylhexanoate |
| | 5.0 | Isopropyl Myristate |
| | 7.0 | Mineral Oil |
| | 0.5 | Shea Butter (Butyrospermum Parkii) |
| | 0.5 | Aluminum Stearate |
| | 0.5 | Magnesium Stearate |
| | 0.2 | Bisabolol |
| | 0.7 | Quaternium-18-Hectorite |
| B | 5.0 | Dipropylene Glycol |
| | 0.7 | Magnesium Sulfate |
| | 1.0 | Resilin protein |
| | q.s. | Preservative |
| | 62.9 | Aqua dem. |
| C | q.s. | Perfume Oil |

### Al 5%

| | % | Ingredient (INCI) |
|---|---|---|
| A | 6.0 | PEG-7 Hydrogenated Castor Oil |
| | 10.0 | Cetearyl Ethylhexanoate |
| | 5.0 | Isopropyl Myristate |
| | 7.0 | Mineral Oil |
| | 0.5 | Shea Butter (Butyrospermum Parkii) |
| | 0.5 | Aluminum Stearate |
| | 0.5 | Magnesium Stearate |
| | 0.2 | Bisabolol |
| | 0.7 | Quaternium-18-Hectorite |
| B | 5.0 | Dipropylene Glycol |
| | 0.7 | Magnesium Sulfate |
| | 5.0 | Resilin protein |
| | q.s. | Preservative |
| | 58.9 | Aqua dem. |
| C | q.s. | Perfume Oil |

Preparation: Heat phases A and B separately to about 80° C. Stir phase B into phase A and homogenize. Cool to about 40° C. with stirring, add phase C and homogenize again. Allow to cool to room temperature with stirring.

### Example 33

### Liquid Make-Up—O/W Type

### Al 1%

| | % | Ingredient (INCI) |
|---|---|---|
| A | 2.0 | Ceteareth-6, Stearyl Alcohol |
| | 2.0 | Ceteareth-25 |
| | 6.0 | Glyceryl Stearate |
| | 1.0 | Cetyl Alcohol |
| | 8.0 | Mineral Oil |
| | 7.0 | Cetearyl Ethylhexanoate |
| | 0.2 | Dimethicone |
| B | 3.0 | Propylene Glycol |
| | 1.0 | Panthenol |
| | q.s. | Preservative |
| | 1.0 | Resilin protein |
| | 61.9 | Aqua dem. |
| C | 0.1 | Bisabolol |
| | q.s. | Perfume Oil |
| D | 5.7 | C.I. 77 891, Titanium Dioxide |
| | 1.1 | Iron Oxides |

### Al 5%

| | % | Ingredient (INCI) |
|---|---|---|
| A | 2.0 | Ceteareth-6, Stearyl Alcohol |
| | 2.0 | Ceteareth-25 |
| | 6.0 | Glyceryl Stearate |
| | 1.0 | Cetyl Alcohol |
| | 8.0 | Mineral Oil |
| | 7.0 | Cetearyl Ethylhexanoate |
| | 0.2 | Dimethicone |
| B | 3.0 | Propylene Glycol |
| | 1.0 | Panthenol |
| | 5.0 | Resilin protein |
| | q.s. | Preservative |
| | 57.9 | Aqua dem. |
| C | 0.1 | Bisabolol |
| | q.s. | Perfume Oil |
| D | 5.7 | C.I. 77 891, Titanium Dioxide |
| | 1.1 | Iron Oxides |

Preparation: Heat phases A and B separately to about 80° C. Stir phase B into phase A and homogenize. Cool to about 40° C. with stirring, add phases C and D and thoroughly homogenize again. Allow to cool to room temperature with stirring.

### Example 34

Dermocosmetic preparations according to the invention are described below, comprising the resilin protein prepared according to Example 1. The following data are parts by weight of an aqueous solution.

Clear shampoo

| Ingredients (INCI) | 1 | 2 | 3 | 4 | 5 |
|---|---|---|---|---|---|
| Sodium Laureth Sulfate | 13.00 | 15.00 | 10.50 | 12.50 | 10.00 |
| Cocamidopropyl Betaine | 7.50 | 7.00 | 5.00 | 5.50 | 10.00 |
| PEG-7 Glyceryl Cocoate | 2.00 | 2.50 | 3.50 | 5.00 | 2.30 |
| Perfume Oil | 0.10 | 0.10 | 0.10 | 0.10 | 0.10 |
| Resilin protein | 1.0 | 5.0 | 0.1 | 0.5 | 10.0 |
| D-Panthenol USP | 1.00 | 1.50 | 1.80 | 1.70 | 1.40 |
| Preservative | 0.10 | 0.10 | 0.10 | 0.10 | 0.10 |
| Citric Acid | 0.10 | 0.10 | 0.10 | 0.10 | 0.10 |
| Luviquat ® Ultra Care | 1.50 | 1.00 | 1.50 | 1.20 | 1.10 |
| Sodium Chloride | 1.50 | 1.40 | 1.40 | 1.30 | 1.50 |
| Aqua dem. | ad 100 | ad 100 | ad 100 | ad 100 | ad 100 |

Shampoo

| Ingredients (INCI) | 1 | 2 | 3 | 4 | 5 |
|---|---|---|---|---|---|
| Sodium Laureth Sulfate | 35.00 | 40.00 | 30.00 | 45.00 | 27.00 |
| Decyl Glucoside | 5.00 | 5.50 | 4.90 | 3.50 | 7.00 |
| Cocamidopropyl Betaine | 10.00 | 5.00 | 12.50 | 7.50 | 15.00 |
| Perfume Oil | 0.10 | 0.10 | 0.10 | 0.10 | 0.10 |
| Resilin protein | 1.0 | 5.0 | 0.1 | 0.5 | 10.0 |
| D-Panthenol USP | 0.50 | 1.00 | 0.80 | 1.50 | 0.50 |
| Preservative | 0.10 | 0.10 | 0.10 | 0.10 | 0.10 |
| Citric Acid | 0.10 | 0.10 | 0.10 | 0.10 | 0.10 |
| Laureth-3 | 0.50 | 2.00 | 0.50 | 0.50 | 2.00 |
| Sodium Chloride | 1.50 | 1.50 | 1.50 | 1.50 | 1.50 |
| Aqua dem. | ad 100 | ad 100 | ad 100 | ad 100 | ad 100 |

Clear conditioner shampoo

| Ingredients (INCI) | 1 | 2 | 3 | 4 | 5 |
|---|---|---|---|---|---|
| ® Disodium | 10.00 | 15.00 | 20.00 | 12.00 | 17.00 |
| Cocoamphodiacetate | | | | | |
| ® Decyl Glucoside | 5.00 | 6.00 | 7.00 | 8.00 | 4.00 |
| ® Cocamidopropyl | 15.00 | 12.00 | 10.00 | 18.00 | 20.00 |
| Betaine | | | | | |
| Luviquat ® FC 550 | 0.30 | 0.20 | 0.20 | 0.20 | 0.30 |
| Perfume Oil | 0.10 | 0.10 | 0.10 | 0.10 | 0.10 |
| Resilin protein | 20.0 | 5.0 | 1.0 | 0.5 | 10.0 |
| Cremophor ® PS 20 | 5.00 | 1.00 | 1.00 | 7.00 | 5.00 |
| Preservative | 0.10 | 0.10 | 0.10 | 0.10 | 0.10 |
| ® Laureth-3 | 2.00 | 1.00 | 0.50 | 2.00 | 2.00 |
| Citric Acid | 0.20 | 0.20 | 0.20 | 0.20 | 0.20 |
| PEG-12 Distearate | 3.00 | 2.00 | 2.00 | 3.00 | 2.50 |
| Aqua dem. | ad 100 | ad 100 | ad 100 | ad 100 | ad 100 |

Foam O/W emulsions

| | Emulsion 1 | | Emulsion 2 | |
|---|---|---|---|---|
| | | % by | | % by |
| | % by wt. | vol. | % by wt. | vol. |
| Stearic Acid | 5.00 | | 1.00 | |
| Cetyl Alcohol | 5.50 | | | |
| Cetearyl Alcohol | | | 2.00 | |
| PEG-40 Stearate | 8.50 | | | |
| PEG-20 Stearate | | | 1.00 | |
| Caprylic/Capric Triglyceride | 4.00 | | 2.00 | |
| C12-15 Alkyl Benzoate | 10.00 | | 15.00 | |
| Cyclomethicone | 4.00 | | | |
| Dimethicone | | | 0.50 | |
| Resilin protein | 5.0 | | 10.0 | |
| Ethylhexyl Isostearate | | | 5.00 | |
| Myristyl Myristate | | | 2.00 | |
| Ceresin | 1.50 | | | |
| Glycerin | | | 3.00 | |
| Hydroxypropyl Starch | 1.00 | | 3.50 | |
| Phosphate | | | | |
| BHT | | | 0.02 | |
| Disodium EDTA | 0.50 | | 0.10 | |
| Perfume Oil, Preservative | q.s. | | q.s. | |
| Colorant | q.s. | | q.s. | |
| Potassium Hydroxide | q.s. | | q.s. | |
| Aqua dem. | ad 100 | | ad 100 | |
| | Adjust pH to | | Adjust pH to | |
| | 6.5-7.5 | | 5.0-6.0 | |
| Emulsion 1 | | 70 | | |
| Emulsion 2 | | | | 35 |
| Nitrogen | | 30 | | |
| Propane/Butane | | | | 65 |

Conditioner shampoo with pearlescence

| | 1 | 2 | 3 |
|---|---|---|---|
| Polyquaternium-10 | 0.50 | 0.50 | 0.40 |
| Sodium Laureth Sulfate | 9.00 | 8.50 | 8.90 |
| Codamidopropyl Betaine | 2.50 | 2.60 | 3.00 |
| Uvinul ® MS 40 | 1.50 | 0.50 | 1.00 |
| Resilin protein | 1.0 | 5.0 | 0.5 |
| Pearlescent solution | 2.00 | 2.50 | |
| Disodium EDTA | 0.10 | 0.15 | 0.05 |
| Preservative, Perfume Oil, Thickener | q.s. | q.s. | q.s. |
| Aqua dem. | ad 100 | ad 100 | ad 100 |

Adjust pH to 6.0

Clear conditioner shampoo

| | 1 | 2 | 3 |
|---|---|---|---|
| Polyquaternium-10 | 0.50 | 0.50 | 0.50 |
| Sodium Laureth Sulfate | 9.00 | 8.50 | 9.50 |
| Resilin protein | 5.0 | 0.1 | 3.0 |
| Uvinul M ® 40 | 1.00 | 1.50 | 0.50 |
| Preservative, Perfume Oil, | q.s. | q.s. | q.s. |
| Thickener | | | |
| Aqua dem. | ad 100 | ad 100 | ad 100 |

Adjust pH to 6.0

Clear conditioner shampoo with volume effect

| | 1 | 2 | 3 |
|---|---|---|---|
| Sodium Laureth Sulfate | 10.00 | 10.50 | 11.00 |
| Uvinul ® MC 80 | 2.00 | 1.50 | 2.30 |
| Resilin protein | 10.0 | 0.1 | 0.5 |
| Cocamidopropyl Betaine | 2.50 | 2.60 | 2.20 |
| Disodium EDTA | 0.01 | 0.10 | 0.01 |
| Preservative, Perfume Oil, | q.s. | q.s. | q.s. |
| Thickener | | | |
| Aqua dem. | ad 100 | ad 100 | ad 100 |

Adjust pH to 6.0

Gel cream

| | 1 | 2 | 3 | 4 |
|---|---|---|---|---|
| Acrylates/C10-30 Alkylacrylate Crosspolymer | 0.40 | 0.35 | 0.40 | 0.35 |
| Carbomer | 0.20 | 0.22 | 0.20 | 0.22 |
| Xanthan Gum | 0.10 | 0.13 | 0.10 | 0.13 |
| Cetearyl Alcohol | 3.00 | 2.50 | 3.00 | 2.50 |
| C12-15 Alkyl Benzoate | 4.00 | 4.50 | 4.00 | 4.50 |
| Caprylic/Capric Triglyceride | 3.00 | 3.50 | 3.00 | 3.50 |
| Uvinul ® A Plus ™ | 2.00 | 1.50 | 0.75 | 1.00 |
| UvaSorb ® k2A | | 3.00 | | |
| Ethylhexyl Bis-Isopentylbenzoxazolylphenyl | | | | |
| Melamine | | | | |
| Uvinul ® MC 80 | 3.00 | | 1.00 | |
| Bis-Ethylhexyloxyphenol Methoxyphenyl Triazine | | 1.50 | | 2.00 |
| Butyl Methoxydibenzoylmethane | | | 2.00 | |
| Disodium Phenyl Dibenzimidazole Tetrasulfonate | 2.50 | | 0.50 | 2.00 |
| Uvinul ® T 150 | 4.00 | | 3.00 | 4.00 |
| Octocrylene | | 4.00 | | |
| Diethylhexyl Butamido Triazone | 1.00 | | | 2.00 |
| Phenylbenzimidazole Sulfonic Acid | 0.50 | | 3.00 | |
| Methylene Bis-Benzotriazolyl | 2.00 | | 0.50 | 1.50 |
| Tetramethylbutyl phenol | | | | |
| Ethylhexyl Salicylate | | | 3.00 | |
| Drometrizole Trisiloxane | | | 0.50 | |
| Terephthalidene Dicamphor Sulfonic Acid | | 1.50 | | 1.00 |
| Diethylhexyl 2,6-Naphthalate | 3.50 | 4.00 | 7.00 | 9.00 |
| Titanium Dioxide-microfine | 1.00 | | 3.00 | |
| Zinc Oxide-microfine | | | | 0.25 |
| Resilin protein | 0.1 | 0.5 | 1.0 | 0.02 |
| Cyclomethicone | 5.00 | 5.50 | 5.00 | 5.50 |
| Dimethicone | 1.00 | 0.60 | 1.00 | 0.60 |
| Glycerin | 1.00 | 1.20 | 1.00 | 1.20 |
| Sodium Hydroxide | q.s. | q.s. | q.s. | q.s. |
| Preservative | 0.30 | 0.23 | 0.30 | 0.23 |
| Perfume Oil | 0.20 | | 0.20 | |
| Aqua dem. | ad 100 | ad 100 | ad 100 | ad 100 |

Adjust pH to 6.0

OW sunscreen formulation

| | 1 | 2 | 3 | 4 | 5 | 6 | 7 |
|---|---|---|---|---|---|---|---|
| Glyceryl Stearate SE | 0.50 | 1.00 | 3.00 | | | 1.50 | |
| Glycerl Stearate Citrate | 2.00 | | 1.00 | 2.00 | 4.00 | | |
| Stearic Acid | | 3.00 | | 2.00 | | | |
| PEG-40 Stearate | 0.50 | | | | | 2.00 | |
| Cetyl Phosphate | | | | | | 1.00 | |
| Sodium Cetearyl Sulfate | | | | | | | 0.75 |
| Stearyl Alcohol | | | 3.00 | | | 2.00 | 0.60 |
| Cetyl Alcohol | 2.50 | 1.10 | | 1.50 | 0.60 | | 2.00 |
| Resilin protein | 10.0 | 0.5 | 3.0 | 5.0 | 0.1 | 0.02 | 7.5 |
| Uvinul ® A Plus ™ | 2.00 | 1.50 | 0.75 | 1.00 | 2.10 | 4.50 | 5.00 |
| UvaSorb ® k2A | | | | | | | |
| Ethylhexyl Bis- | | | | | | | |
| Isopentylbenzoxazolylphenyl | | | | | | | |
| Melamine | | | | | | | |
| Ethylhexyl Methoxycinnamate | | | | | 5.00 | 6.00 | 8.00 |
| Uvinul ® MC 80 | | | | | | | |
| Bis-Ethylhexyloxyphenol | | 1.50 | | 2.00 | 2.50 | | 2.50 |
| Methoxyphenyl Triazine | | | | | | | |
| Butyl Methoxydibenzoylmethane | | | 2.00 | | 2.00 | 1.50 | |
| Disodium Phenyl Dibenzimidazole | 2.50 | | 0.50 | 2.00 | | 0.30 | |
| Tetrasulfonate | | | | | | | |
| Ethyhexyl Triazone Uvinul ® T 150 | 4.00 | | 3.00 | 4.00 | | 2.00 | |
| Octocrylene | | 4.00 | | | | | 7.50 |
| Diethylhexyl Butamido Triazone | 1.00 | | | 2.00 | 1.00 | | 1.00 |
| Phenylbenzimidazole Sulfonic Acid | 0.50 | | 3.00 | | | | |
| Methylene Bis-Benzotriazolyl | 2.00 | | 0.50 | 1.50 | 2.50 | | |
| Tetramethylbutylphenol | | | | | | | |
| Ethylhexyl Salicylate | | | 3.00 | | | | 5.00 |
| Drometrizole Trisiloxane | | | 0.50 | | | 1.00 | |
| Terephthalidene Dicamphor | | 1.50 | | 1.00 | 1.00 | | 0.50 |
| Sulfonic Acid | | | | | | | |
| Diethylhexyl 2,6-Naphthalate | 3.50 | | 7.00 | | 6.00 | 9.00 | |
| Titanium Dioxide-microfine | 1.00 | | 3.00 | | 3.50 | | 1.50 |
| Zinc Oxide-microfine | | | | 0.25 | | 2.00 | |
| C12-15 Alkyl Benzoate | | 0.25 | | | 4.00 | 7.00 | |
| Dicapryl Ether | | | 3.50 | | 2.00 | | |
| Butylene Glycol | 5.00 | | 6.00 | | | | |
| Dicaprylate/Dicaprate | | | | | | | |
| Cocoglyceride | | | 6.00 | | 2.00 | | |
| Dimethicone | 0.50 | | 1.00 | | 2.00 | | |
| Cyclomethlcone | 2.00 | | 0.50 | | 0.50 | | |
| Butyrospermum Parkii (Shea | | 2.00 | | | | | |
| Butter) | | | | | | | |
| VP/Hexadecene Copolymer | 0.20 | | | 0.50 | | 1.00 | |
| Glycerin | 3.00 | 7.50 | | 7.50 | 5.00 | | 2.50 |
| Xanthan Gum | 0.15 | | 0.05 | | | 0.30 | |
| Sodium Carbomer | | 0.20 | | 0.15 | 0.25 | | |
| Vitamin E Acetate | 0.60 | | 0.23 | | 0.70 | 1.00 | |
| Biosaccaride Gum-1 | | 3.00 | 10.00 | | | | |
| Glycine Soya (Soybean) Oil | | | | 0.50 | | 1.50 | 1.00 |
| Ethylhexylglycerin | 0.30 | | | | | | |
| DMDM Hydantoin | | 0.60 | 0.40 | 0.20 | | | |
| Iodopropynyl Butylcarbamate | | | | 0.18 | 0.20 | | |
| Methylparaben | 0.15 | | 0.25 | | 0.50 | | |
| Phenoxyethanol | 1.00 | 0.40 | | | 0.40 | 0.50 | 0.40 |
| Trisodium EDTA | 0.02 | | 0.05 | | | | |
| Tetrasodium Iminodisuccinate | | | | 0.25 | 1.00 | | |
| Ethanol | 2.00 | 1.50 | | 3.00 | | 1.20 | 5.00 |
| Perfume Oil | 0.10 | 0.25 | 0.30 | | 0.40 | 0.20 | |
| Aqua dem. | ad 100 | ad 100 | ad 100 | ad 100 | ad 100 | ad 100 | ad 100 |

Hydrodispersion

| | 1 | 2 | 3 | 4 | 5 |
|---|---|---|---|---|---|
| Ceteaereth-20 | 1.00 | | | 0.50 | |
| Cetyl Alcohol | | | 1.00 | | |
| Sodium Carbomer | | 0.20 | | 0.30 | |
| Acrylates/C10-30 Alkyl Acrylate | 0.50 | | 0.40 | 0.10 | 0.50 |
| Crosspolymer | | | | | |
| Xanthan Gum | | 0.30 | 0.15 | | |
| Resilin protein | 5.0 | 0.5 | 3.0 | 0.1 | 10.0 |
| Uvinul ® A Plus ™ | 2.00 | 1.50 | 0.75 | 1.00 | 2.10 |
| UvaSorb ® k2A Ethylhexyl Bis- | | 3.50 | | | |
| Isopentylbenzoxazolylphenyl Melamine | | | | | |
| Ethylhexyl Methoxycinnamate Uvinul ® | | | | | 5.00 |
| MC 80 | | | | | |
| Bis-Ethylhexyloxyphenol Methoxyphenyl | | 1.50 | | 2.00 | 2.50 |
| Triazine | | | | | |
| Butyl Methoxydibenzoylmethane | | | 2.00 | | 2.00 |
| Disodium Phenyl Dibenzimidazole | 2.50 | | 0.50 | 2.00 | |
| Tetrasulfonate | | | | | |
| Ethyhexyl Triazone Uvinul ® T 150 | 4.00 | | 3.00 | 4.00 | |
| Octocrylene | | 4.00 | | | |
| Diethylhexyl Butamido Triazone | 1.00 | | | 2.00 | 1.00 |
| Phenylbenzimidazole Sulfonic Acid | 0.50 | | 3.00 | | |
| Methylene Bis-Benzotriazolyl | 2.00 | | 0.50 | 1.50 | 2.50 |
| Tetramethylbutylphenol | | | | | |
| Ethylhexyl Salicylate | | | 3.00 | | |
| Drometrizole Trisiloxane | | | 0.50 | | |
| Terephthalidene Dicamphor Sulfonic Acid | | 1.50 | | 1.00 | 1.00 |
| Diethylhexyl 2,6-Naphthalate | | | 7.00 | | 9.00 |
| Titanium Dioxide-microfine | 1.00 | | 3.00 | | 3.50 |
| Zinc Oxide-microfine | | | | 0.25 | |
| C12-15 Alkyl Benzoate | 2.00 | 2.50 | | | |
| Dicapryl Ether | | 4.00 | | | |
| Butylene glycol Dicaprylate/Dicaprate | 4.00 | | 2.00 | 6.00 | |
| Dicapryl Carbonate | | 2.00 | 6.00 | | |
| Dimethicone | | 0.50 | 1.00 | | |
| Phenyl Trimethicone | 2.00 | | 0.50 | | |
| Butyrospermum Parkii (Shea Butter) | | 2.00 | | 5.00 | |
| VP/Hexadecene Copolymer | 0.50 | | | 0.50 | 1.00 |
| Tricontanyl PVP | 0.50 | | 1.00 | | |
| Ethylhexylglycerin | | | 1.00 | | 0.80 |
| Glycerin | 3.00 | 7.50 | | 7.50 | 8.50 |
| Glycine Soya (Soybean) Oil | | | 1.50 | | 1.00 |
| Vitamin E Acetate | 0.50 | | 0.25 | | 1.00 |
| Glucosylrutin | 0.60 | | | 0.25 | |
| Biosaccaride Gum-1 | | 2.50 | 0.50 | | 2.00 |
| DMDM Hydantoin | | 0.60 | 0.45 | 0.25 | |
| Iodopropynyl Butylcarbamate | 0.20 | | | | |
| Methylparaben | 0.50 | | 0.25 | 0.15 | |
| Phenoxyethanol | 0.50 | 0.40 | | 1.00 | |
| Trisodium EDTA | | 0.01 | 0.05 | | 0.10 |
| Ethanol | 3.00 | 2.00 | 1.50 | | 7.00 |
| Perfume Oil | 0.20 | | 0.05 | 0.40 | |
| Aqua dem. | ad 100 | ad 100 | ad 100 | ad 100 | ad 100 |

WO Sunscreen Emulsion

| | 1 | 2 | 3 | 4 | 5 |
|---|---|---|---|---|---|
| Cetyl Dimethicone | | 2.50 | | 4.00 | |
| Polyglyceryl-2 Dipolyhydroxystearate | 5.00 | | | | 4.50 |
| PEG-30 Dipolyhydroxystearate | | | 5.00 | | |
| Resilin protein | 5.0 | 1.0 | 10.0 | 0.5 | 0.1 |
| Uvinul ® A Plus ™ | 2.00 | 1.50 | 0.75 | 1.00 | 2.10 |
| UvaSorb ® k2A | | 2.00 | | | |
| Ethylhexyl Bis-Isopentylbenzoxazolylphenyl | | | | | |
| Melamine | | | | | |
| Ethylhexyl Methoxycinnamate Uvinul ® | | | | | 5.00 |
| MC 80 | | | | | |
| Bis-Ethylhexyloxyphenol Methoxyphenyl | | 1.50 | | 2.00 | 2.50 |
| Triazine | | | | | |
| Butyl Methoxydibenzoylmethane | | | 2.00 | | 2.00 |
| Disodium Phenyl Dibenzimidazole | 2.50 | | 0.50 | 2.00 | |
| Tetrasulfonate | | | | | |
| Ethyhexyl Triazone Uvinul ® 150 | 4.00 | | 3.00 | 4.00 | |
| Octocrylene | | 4.00 | | | |
| Diethylhexyl Butamido Triazone | 1.00 | | | 2.00 | 1.00 |
| Phenylbenzimidazole Sulfonic Acid | 0.50 | | 3.00 | | |
| Methylene Bis-Benzotriazolyl | 2.00 | | 0.50 | 1.50 | 2.50 |
| Tetramethylbutylphenol | | | | | |
| Ethylhexyl Salicylate | | | 3.00 | | |
| Drometrizole Trisiloxane | | | 0.50 | | |
| Terephthalidene Dicamphor Sulfonic Acid | | 1.50 | | 1.00 | 1.00 |
| Diethylhexyl 2,6-Naphthalate | | | 7.00 | | 4.00 |
| Titanium Dioxide-microfine | 1.00 | | 3.00 | | 3.50 |
| Zinc Oxide-microfine | | | | 0.25 | |
| Mineral Oil | | 12.00 | 10.00 | | 8.00 |
| C12-15 Alkyl Benzoate | | | | 9.00 | |
| Dicaprylyl Ether | 10.00 | | | | 7.00 |
| Butylene glycol Dicaprylate/Dicaprate | | | 2.00 | 8.00 | 4.00 |
| Dicaprylyl Carbonate | 5.00 | | 6.00 | | |
| Dimethicone | | 4.00 | 1.00 | 5.00 | |
| Cyclomethicone | 2.00 | 25.00 | | | 2.00 |
| Butyrospermum Parkii (Shea Butter) | | | 3.00 | | |
| Petrolatum | | 4.50 | | | |
| VP/Hexadecene Copolymer | 0.50 | | | 0.50 | 1.00 |
| Ethylhexylglycerin | | 0.30 | 1.00 | | 0.50 |
| Glycerin | 3.00 | 7.50 | | 7.50 | 8.50 |
| Glycine Soja (Soybean) Oil | | 1.00 | 1.50 | | 1.00 |
| Magnesium Sulfate MgSO4 | 1.00 | 0.50 | | 0.50 | |
| Magnesium Chloride MgCl2 | | | 1.00 | | 0.70 |
| Vitamin E Acetate | 0.50 | | 0.25 | | 1.00 |
| Ascorbyl Palmitate | 0.50 | | | 2.00 | |
| Biosaccaride Gum-1 | | | | 3.50 | 1.00 |
| DMDM Hydantoin | | 0.60 | 0.40 | 0.20 | |
| Methylparaben | 0.50 | | 0.25 | 0.15 | |
| Phenoxyethanol | 0.50 | 0.40 | | 1.00 | |
| Trisodium EDTA | 0.12 | 0.05 | | 0.30 | |
| Ethanol | 3.00 | | 1.50 | | 5.00 |
| Perfume Oil | 0.20 | | 0.40 | 0.35 | |
| Aqua dem. | ad 100 | ad 100 | ad 100 | ad 100 | ad 100 |

Sticks

| | 1 | 2 | 3 | 4 |
|---|---|---|---|---|
| Caprylic/Capric Triglyceride | 12.00 | 10.00 | 6.00 | |
| Octyldodecanol | 7.00 | 14.00 | 8.00 | 3.00 |
| Butylene Glycol Dicaprylate/ | | | | 12.00 |
| Dicaprate | | | | |
| Pentaerythrityl Tetraisostearate | 10.00 | 6.00 | 8.00 | 7.00 |
| Polyglyceryl-3 Diisostearate | 2.50 | | | |
| Bis-Diglyceryl | 9.00 | 8.00 | 10.00 | 8.00 |
| Polyacyladipate-2 | | | | |
| Cetearyl Alcohol | 8.00 | 11.00 | 9.00 | 7.00 |
| Myristyl Myristate | 3.50 | 3.00 | 4.00 | 3.00 |
| Beeswax | 5.00 | 5.00 | 6.00 | 6.00 |
| Copernicia Cerifera (Carnauba) | 1.50 | 2.00 | 2.00 | 1.50 |
| Wax | | | | |
| Cera Alba | 0.50 | 0.50 | 0.50 | 0.40 |
| C16-40 Alkyl Stearate | | 1.50 | 1.50 | 1.50 |
| Resilin protein | 0.5 | 3.0 | 1.0 | 5.0 |
| Uvinul ® A Plus ™ | 2.00 | 1.50 | 0.75 | 9.00 |
| UvaSorb ® k2A | | 2.00 | | 4.00 |
| Ethylhexyl Bis- | | | | |
| Isopentylbenzoxazolylphenyl | | | | |
| Melamine | | | | |
| Ethylhexyl Methoxycinnamate | | 3.00 | | |
| Uvinul ® MC 80 | | | | |
| Bis-Ethylhexyloxyphenol | | 1.50 | | 2.00 |
| Methoxyphenyl Triazine | | | | |
| Butyl Methoxydibenzoylmethane | | | 2.00 | |
| Disodium Phenyl Dibenzimidazole | 2.50 | | 0.50 | 2.00 |
| Tetrasulfonate | | | | |
| Ethylhexyl Triazone | 4.00 | | 3.00 | 4.00 |
| Uvinul ® T 150 | | | | |
| Octocrylene | | 4.00 | | |
| Diethylhexyl Butamido Triazone | 1.00 | | | 2.00 |
| Phenylbenzimidazole Sulfonic | 0.50 | | 3.00 | |
| Acid | | | | |
| Methylene Bis-Benzotriazolyl | 2.00 | | 0.50 | 1.50 |
| Tetramethylbutylphenol | | | | |
| Ethylhexyl Salicylate | | | 3.00 | |
| Drometrizole Trisiloxane | | | 0.50 | |
| Terephthalidene Dicamphor | | 1.50 | | 1.00 |
| Sulfonic Acid | | | | |
| Diethylhexyl 2,6-Naphthalate | | | 7.00 | |
| Titanium Dioxide-microfine | 1.00 | | 3.00 | |
| Zinc Oxide-microfine | | | | 0.25 |
| Vitamin E Acetate | 0.50 | 1.00 | | |
| Ascorbyl Palmitate | 0.05 | | 0.05 | |
| Buxux Chinensis (Jojoba) Oil | 2.00 | 1.00 | | 1.00 |
| Perfume Oil, BHT | 0.10 | 0.25 | | 0.35 |
| Ricinus Communis | ad 100 | ad 100 | ad 100 | ad 100 |
| (Castor) Oil | | | | |

PIT-Emulsion

| | 1 | 2 | 3 | 4 | 5 | 6 | 7 | 8 |
|---|---|---|---|---|---|---|---|---|
| Glyceryl Monostearate SE | 0.50 | 2.00 | 3.00 | 5.00 | | 0.50 | 4.00 | |
| Glyceryl Isostearate | | | | | 3.50 | 4.00 | 2.00 | |
| Isoceteth-20 | | 0.50 | | | 2.00 | | | |
| Ceteareth-12 | | 5.00 | | 1.00 | | | 3.50 | 5.00 |
| Ceteareth-20 | | 5.00 | | 1.00 | | | | 3.50 |
| PEG-100 Stearate | | | | 2.80 | | 2.30 | 3.30 | |
| Cetyl Alcohol | 5.20 | | 1.20 | 1.00 | 1.30 | | 0.50 | 0.30 |
| Cetyl Palmitate | 2.50 | 1.20 | | 1.50 | | 0.50 | | 1.50 |
| Cetyl Dimethicone | | | | 0.50 | | 1.00 | | |
| Copolyol | | | | | | | | |
| Polyglyceryl-2-Dioleate | | | | 0.75 | 0.30 | | | |
| Resilin protein | 0.1 | 5.0 | 0.01 | 0.5 | 3.0 | 0.25 | 10.0 | 3.0 |
| Uvinul ® A Plus ™ | 2.00 | 1.50 | 0.75 | 1.00 | 2.10 | 4.50 | 5.00 | 2.10 |
| UvaSorb ® k2A | | | 4.00 | | | | 1.50 | |
| Ethylhexyl Bis- | | | | | | | | |
| Isopentylbenzoxazolyl- | | | | | | | | |
| phenyl Melamine | | | | | | | | |
| Ethylhexyl | | | | | 5.00 | 6.00 | 8.00 | 5.00 |
| Methoxycinnamate | | | | | | | | |
| Uvinul ® MC 80 | | | | | | | | |
| Bis-Ethylhexyloxyphenol | | 1.50 | | 2.00 | 2.50 | | 2.50 | 2.50 |
| Methoxyphenyl Triazine | | | | | | | | |
| Butyl Methoxydibenzoyl- | | | 2.00 | | 2.00 | 1.50 | | 2.00 |
| methane | | | | | | | | |
| Disodium Phenyl | 2.50 | | 0.50 | 2.00 | | 0.30 | | |
| Dibenzimidazole | | | | | | | | |
| Tetrasulfonate | | | | | | | | |
| Ethyhexyl Triazone | 4.00 | | 3.00 | 4.00 | | 2.00 | | |
| Uvinul ® T 150 | | | | | | | | |
| Octocrylene | | 4.00 | | | | | 7.50 | |
| Diethylhexyl Butamido | 1.00 | | | 2.00 | 1.00 | | 1.00 | 1.00 |
| Triazone | | | | | | | | |
| Phenylbenzimidazole | 0.50 | | 3.00 | | | | | |
| Sulfonic Acid | | | | | | | | |
| Methylene Bis- | 2.00 | | 0.50 | 1.50 | 2.50 | | | 2.50 |
| Benzotriazolyl | | | | | | | | |
| Tetramethylbutylphenol | | | | | | | | |
| Ethylhexyl Salicylate | | | 3.00 | | | | 5.00 | |
| Drometrizole Trisiloxane | | | 0.50 | | | 1.00 | | |
| Terephthalylidene | | 1.50 | | 1.00 | 1.00 | | 0.50 | 1.00 |
| Dicamphor Sulfonic Acid | | | | | | | | |
| Diethylhexyl | | | 7.00 | | 10.00 | 7.50 | | 8.00 |
| 2,6-Naphthalate | | | | | | | | |
| TitaniumDioxide - | 1.00 | | 3.00 | | 3.50 | | 1.50 | 3.50 |
| microfine | | | | | | | | |
| Zinc Oxide - microfine | | | | 0.25 | | 2.00 | | |
| C12-15 Alkyl Benzoate | 3.50 | | | 6.35 | | | | 0.10 |
| Cocoglyceride | | 3.00 | | 3.00 | | | | 1.00 |
| Dicapryl Ether | 4.50 | | | | | | | |
| Dicaprylyl Carbonate | | 4.30 | | 3.00 | | | | 7.00 |
| Dibutyl Adipate | | | | 0.50 | | | | 0.30 |
| Phenyl Trimethicone | 2.00 | | | 3.50 | | 2.00 | | |
| Cyclomethicone | | 3.00 | | | | | | |
| C1-5 Alkyl | | 0.50 | | | 2.00 | | | |
| Galactomannan | | | | | | | | |
| Hydrogenated Coco- | | | | | 3.00 | 4.00 | | |
| Glycerides | | | | | | | | |
| Behenoxy Dimethicone | | | | | | 1.50 | 2.00 | |
| VP/Hexadecene | | | | 1.00 | 1.20 | | | |
| Copolymer | | | | | | | | |
| Glycerin | 4.00 | 6.00 | 5.00 | | 8.00 | 10.00 | | |
| Vitamin E Acetate | 0.20 | 0.30 | 0.40 | | 0.30 | | | |
| Butyrospermum Parkii | | 2.00 | | 3.60 | | 2.00 | | |
| (Shea Butter) | | | | | | | | |
| Iodopropyl | 0.12 | | | | 0.20 | | | |
| Butylcarbamate | | | | | | | | |
| Biosaccaride Gum-1 | | | | 0.10 | | | | |
| DMDM Hydantoin | 0.10 | | | | 0.12 | | 0.13 | |
| Methylparaben | | 0.50 | 0.30 | | 0.35 | | | |
| Phenoxyethanol | 0.50 | 0.40 | | 1.00 | | | | |
| Ethylhexylglycerin | | 0.30 | | | 1.00 | | 0.35 | |
| Ethanol | 2.00 | | 2.00 | | | 5.00 | | |
| Trisodium EDTA | 0.40 | | 0.15 | | | 0.20 | | |
| Perfume Oil | 0.20 | | 0.20 | | 0.24 | 0.16 | 0.10 | 0.10 |
| Aqua dem. | ad 100 | ad 100 | ad 100 | ad 100 | ad 100 | ad 100 | ad 100 | ad |
| | | | | | | | | 100 |

Gel cream

| | 1 | 2 | 3 | 4 |
|---|---|---|---|---|
| Acrylates/C10-30 Alkylacrylate | 0.40 | 0.35 | 0.40 | 0.35 |
| Crosspolymer | | | | |
| Carbomer | 0.20 | 0.22 | 0.20 | 0.22 |
| Luvigel ® EM | 1.50 | 2.50 | 2.80 | 3.50 |
| Xanthan Gum | 0.10 | 0.13 | 0.10 | 0.13 |
| Cetearyl Alcohol | 3.00 | 2.50 | 3.00 | 2.50 |
| C12-15 Alkyl benzoate | 4.00 | 4.50 | 4.00 | 4.50 |
| Caprylic/Capric Triglyceride | 3.00 | 3.50 | 3.00 | 3.50 |
| Titanium Dioxide-microfine | 1.00 | | 1.50 | |
| Zinc Oxide-microfine | | 2.00 | | 0.25 |
| Resilin protein | 0.5 | 10.0 | 3.0 | 5.0 |
| Dihydroxyacetone | | | 3.00 | 5.00 |
| Cyclomethicone | 5.00 | 5.50 | 5.00 | 5.50 |
| Dimethicone | 1.00 | 0.60 | 1.00 | 0.60 |
| Glycerin | 1.00 | 1.20 | 1.00 | 1.20 |
| Sodium Hydroxide | q.s. | q.s. | q.s. | q.s. |
| Preservative | 0.30 | 0.23 | 0.30 | 0.23 |
| Perfume Oil | 0.20 | | 0.20 | |
| Aqua dem. | ad 100 | ad 100 | ad 100 | ad 100 |

Adjust pH to 6.0

OW self-tanning formulations

| | 1 | 2 | 3 | 4 | 5 | 6 | 7 |
|---|---|---|---|---|---|---|---|
| Glyceryl Monostearate SE | 0.50 | 1.00 | 3.00 | | | 1.50 | |
| Glyceryl Stearate Citrate | 2.00 | | 1.00 | 2.00 | 4.00 | | |
| Stearic Acid | | 3.00 | | 2.00 | | | |
| PEG-40 Stearate | 0.50 | | | | | 2.00 | |
| Cetyl Phosphate | | | | | | 1.00 | |
| Cetearyl Sulfate | | | | | | | 0.75 |
| Stearyl Alcohol | | | 3.00 | | | 2.00 | 0.60 |
| Cetyl Alcohol | 2.50 | 1.10 | | 1.50 | 0.60 | | 2.00 |
| Resilin protein | 0.1 | 0.5 | 0.025 | 5.0 | 3.0 | 10.0 | 1.0 |
| Dihydroxyacetone | | | 3.00 | 5.00 | | 4 | |
| Titanium Dioxide - microfine | 1.00 | | | | 1.50 | | 1.50 |
| Zinc Oxide - microfine | | | | 0.25 | | 2.00 | |
| C12-15 Alkyl Benzoate | | 0.25 | | | 4.00 | 7.00 | |
| Dicapryl Ether | | | 3.50 | | 2.00 | | |
| Butylene glycol | 5.00 | | 6.00 | | | | |
| Dicaprylate/Dicaprate | | | | | | | |
| Cocoglycerides | | | 6.00 | | 2.00 | | |
| Dimethicone | 0.50 | | 1.00 | | 2.00 | | |
| Cyclomethicone | 2.00 | | 0.50 | | 0.50 | | |
| Butyrospermum Parkii | | 2.00 | | | | | |
| (Shea)Butter | | | | | | | |
| VP/Hexadecene Copolymer | 0.20 | | | 0.50 | | 1.00 | |
| Glycerin | 3.00 | 7.50 | | 7.50 | 5.00 | | 2.50 |
| Xanthan Gum | 0.15 | | 0.05 | | | 0.30 | |
| Sodium Carbomer | | 0.20 | | 0.15 | 0.25 | | |
| Vitamin E Acetate | 0.60 | | 0.23 | | 0.70 | 1.00 | |
| Biosaccaride Gum-1 | | 3.00 | 10.00 | | | | |
| Glycine Soja (Soybean) Oil | | | | 0.50 | | 1.50 | 1.00 |
| Ethylhexylglycerin | 0.30 | | | | | | |
| DMDM Hydantoin | | 0.60 | 0.40 | 0.20 | | | |
| Iodopropynyl Butylcarbamate | | | | 0.18 | 0.20 | | |
| Methylparaben | 0.15 | | 0.25 | | 0.50 | | |
| Phenoxyethanol | 1.00 | 0.40 | | | 0.40 | 0.50 | 0.40 |
| Trisodium EDTA | 0.02 | | 0.05 | | | | |
| Tetrasodium | | | | 0.25 | 1.00 | | |
| Iminodisuccinate | | | | | | | |
| Ethanol | 2.00 | 1.50 | | 3.00 | | 1.20 | 5.00 |
| Perfume Oil | 0.10 | 0.25 | 0.30 | | 0.40 | 0.20 | |
| Aqua dem. | ad 100 | ad 100 | ad 100 | ad 100 | ad 100 | ad 100 | ad 100 |

OW Make-Up

| | 1 | 2 | 3 | 4 | 5 | 6 | 7 |
|---|---|---|---|---|---|---|---|
| Glyceryl Monostearate SE | 0.50 | 1.00 | 3.00 | | | 1.50 | |
| Glycerl Stearate Citrate | 2.00 | | 1.00 | 2.00 | 4.00 | | |
| Stearic Acid | | 3.00 | | 2.00 | | | |
| PEG-40 Stearate | 0.50 | | | | | 2.00 | |
| Cetyl Phosphate | | | | | | 1.00 | |
| Cetearyl Sulfate | | | | | | | 0.75 |
| Stearyl Alcohol | | | 3.00 | | | 2.00 | 0.60 |
| Cetyl Alcohol | 2.50 | 1.10 | | 1.50 | 0.60 | | 2.00 |
| Resilin protein | 3.0 | 5.0 | 2.0 | 0.5 | 1.0 | 5.0 | 10.0 |
| Titanium Dioxide | 10.00 | 12.00 | 9.00 | 8.50 | 11.00 | 9.50 | 10.00 |
| Iron Oxide | 2.00 | 4.00 | 3.00 | 5.00 | 3.40 | 6.00 | 4.40 |
| Zinc Oxide | | 4.00 | | 2.00 | | 3.00 | |
| C12-15 Alkyl Benzoate | | 0.25 | | | 4.00 | 7.00 | |
| Dicapryl Ether | | | 3.50 | | 2.00 | | |
| Butylene glycol | 5.00 | | 6.00 | | | | |
| Dicaprylate/Dicaprate | | | | | | | |
| Cocoglycerides | | | 6.00 | | 2.00 | | |
| Dimethicone | 0.50 | | 1.00 | | 2.00 | | |
| Cyclomethicone | 2.00 | | 0.50 | | 0.50 | | |
| Butyrospermum Parkii | | 2.00 | | | | | |
| (Shea Butter) | | | | | | | |
| VP/Hexadecene Copolymer | 0.20 | | | 0.50 | | 1.00 | |
| Glycerin | 3.00 | 7.50 | | 7.50 | 5.00 | | 2.50 |
| Xanthan Gum | 0.15 | | 0.05 | | | 0.30 | |
| Sodium Carbomer | | 0.20 | | 0.15 | 0.25 | | |
| Vitamin E Acetate | 0.60 | | 0.23 | | 0.70 | 1.00 | |
| Glycine Soja (Soybean) Oil | | | | 0.50 | | 1.50 | 1.00 |
| Ethylhexylglycerin | 0.30 | | | | | | |
| DMDM Hydantoin | | 0.60 | 0.40 | 0.20 | | | |
| Iodopropynyl Butylcarbamate | | | | 0.18 | 0.20 | | |
| Methylparaben | 0.15 | | 0.25 | | 0.50 | | |
| Phenoxyethanol | 1.00 | 0.40 | | | 0.40 | 0.50 | 0.40 |
| Trisodium EDTA | 0.02 | | 0.05 | | | | |
| Tetrasodium | | | | 0.25 | 1.00 | | |
| Iminodisuccinate | | | | | | | |
| Ethanol | 2.00 | 1.50 | | 3.00 | | 1.20 | 5.00 |
| Perfume Oil | 0.10 | 0.25 | 0.30 | | 0.40 | 0.20 | |
| Aqua dem. | ad 100 | ad 100 | ad 100 | ad 100 | ad 100 | ad 100 | ad 100 |

Self-tanning hydrodispersion

| | 1 | 2 | 3 | 4 | 5 |
|---|---|---|---|---|---|
| Ceteareth-20 | 1.00 | | | 0.50 | |
| Cetyl Alcohol | | | 1.00 | | |
| Luvigel ® EM | | 2.00 | | 2.50 | 2.00 |
| Acrylates/C10-30 Alkyl Acrylate | 0.50 | | 0.40 | 0.10 | 0.50 |
| Crosspolymer | | | | | |
| Xanthan Gum | | 0.30 | 0.15 | | |
| Resilin protein | 3.0 | 1.0 | 0.5 | 0.1 | 5.0 |
| Dihydroxyacetone | | | 3.00 | 5.00 | |
| Uvinul ® A Plus ™ | 2.00 | 1.50 | 0.75 | 1.00 | 2.10 |
| Titanium Dioxide - microfine | 1.00 | | 1.00 | | 1.00 |
| Zinc Oxide - microfine | | 1.90 | | 0.25 | |
| C12-15 Alkyl Benzoate | 2.00 | 2.50 | | | |
| Dicapryl Ether | | 4.00 | | | |
| Butylene glycol Dicaprylate/Dicaprate | 4.00 | | 2.00 | 6.00 | |
| Dicapryl Carbonate | | 2.00 | 6.00 | | |
| Dimethicone | | 0.50 | 1.00 | | |
| Phenyl Trimethicone | 2.00 | | 0.50 | | |
| Butyrospermum Parkii (Shea Butter) | | 2.00 | | 5.00 | |
| VP/Hexadecene Copolymer | 0.50 | | | 0.50 | 1.00 |
| Tricontanyl PVP | 0.50 | | 1.00 | | |
| Ethylhexylglycerin | | | 1.00 | | 0.80 |
| Glycerin | 3.00 | 7.50 | | 7.50 | 8.50 |
| Glycine Soja (Soybean) Oil | | | 1.50 | | 1.00 |
| Vitamin E Acetate | 0.50 | | 0.25 | | 1.00 |
| Glucosylrutin | 0.60 | | | 0.25 | |
| DMDM Hydantoin | | 0.60 | 0.45 | 0.25 | |
| Iodopropynyl Butylcarbamate | 0.20 | | | | |
| Methylparaben | 0.50 | | 0.25 | 0.15 | |
| Phenoxyethanol | 0.50 | 0.40 | | 1.00 | |
| Trisodium EDTA | | 0.01 | 0.05 | | 0.10 |
| Ethanol | 3.00 | 2.00 | 1.50 | | 7.00 |
| Perfume Oil | 0.20 | | 0.05 | 0.40 | |
| Aqua dem. | ad 100 | ad 100 | ad 100 | ad 100 | ad 100 |

Aftersun hydrodispersion

| | 1 | 2 | 3 | 4 | 5 |
|---|---|---|---|---|---|
| Ceteareth-20 | 1.00 | | | 0.50 | |
| Cetyl Alcohol | | | 1.00 | | |
| Luvigel ® EM | | 2.00 | | 2.50 | 2.00 |
| Acrylates/C10-30 Alkyl | 0.50 | 0.30 | 0.40 | 0.10 | 0.50 |
| Acrylate Crosspolymer | | | | | |
| Xanthan Gum | | 0.30 | 0.15 | | |
| Resilin protein | 0.1 | 5.0 | 0.5 | 3.0 | 1.0 |
| C12-15 Alkyl Benzoate | 2.00 | 2.50 | | | |
| Dicapryl Ether | | 4.00 | | | |
| Butylene glycol | 4.00 | | 2.00 | 6.00 | |
| Dicaprylate/Dicaprate | | | | | |
| Dicapryl Carbonate | | 2.00 | 6.00 | | |
| Dimethicone | | 0.50 | 1.00 | | |
| Phenyl Trimethicone | 2.00 | | 0.50 | | |
| Tricontanyl PVP | 0.50 | | 1.00 | | |
| Ethylhexylglycerin | | | 1.00 | | 0.80 |
| Glycerin | 3.00 | 7.50 | | 7.50 | 8.50 |
| Glycine Soja (Soybean) | | | 1.50 | | 1.00 |
| Oil | | | | | |
| Vitamin E Acetate | 0.50 | | 0.25 | | 1.00 |
| Glucosylrutin | 0.60 | | | 0.25 | |
| Trisodium EDTA | | 0.01 | 0.05 | | 0.10 |
| Ethanol | 15.00 | 10.00 | 8.00 | 12.00 | 9.00 |
| Perfume Oil | 0.20 | | 0.05 | 0.40 | |
| Aqua dem. | ad 100 | ad 100 | ad 100 | ad 100 | ad 100 |

WO Emulsions

| | 1 | 2 | 3 | 4 | 5 |
|---|---|---|---|---|---|
| Cetyl Dimethicone | | 2.50 | | 4.00 | |
| Polyglyceryl-2 Dipolyhydroxystearate | 5.00 | | | | 4.50 |
| PEG-30 Dipolyhydroxystearate | | | 5.00 | | |
| Resilin protein | 5.0 | 10.0 | 0.1 | 0.5 | 1.0 |
| Uvinul ® A Plus ™ | 2.00 | 1.50 | 0.75 | 1.00 | 2.10 |
| Titanium Dioxide- microfine | 1.00 | | 3.00 | | 3.50 |
| Zinc Oxide- microfine | | 0.90 | | 0.25 | |
| Mineral Oil | | 12.00 | 10.00 | | 8.00 |
| C12-15 Alkyl Benzoate | | | | 9.00 | |
| Dicaprylyl Ether | 10.00 | | | | 7.00 |
| Butylene glycol Dicaprylate/Dicaprate | | | 2.00 | 8.00 | 4.00 |
| Dicaprylyl Carbonate | 5.00 | | 6.00 | | |
| Dimethicone | | 4.00 | 1.00 | 5.00 | |
| Cyclomethicone | 2.00 | 25.00 | | | 2.00 |
| Butyrospermum Parkii (Shea Butter) | | | 3.00 | | |
| Petrolatum | | 4.50 | | | |
| VP/Hexadecene Copolymer | 0.50 | | | 0.50 | 1.00 |
| Ethylhexylglycerin | | 0.30 | 1.00 | | 0.50 |
| Glycerin | 3.00 | 7.50 | | 7.50 | 8.50 |
| Glycine Soja (Soybean) Oil | | 1.00 | 1.50 | | 1.00 |
| Magnesium Sulfate | 1.00 | 0.50 | | 0.50 | |
| Magnesium Chloride | | | 1.00 | | 0.70 |
| Vitamin E Acetate | 0.50 | | 0.25 | | 1.00 |
| Ascorbyl Palmitate | 0.50 | | | 2.00 | |
| Biosaccaride Gum-1 | | | | 3.50 | 7.00 |
| DMDM Hydantoin | | 0.60 | 0.40 | 0.20 | |
| Methylparaben | 0.50 | | 0.25 | 0.15 | |
| Phenoxyethanol | 0.50 | 0.40 | | 1.00 | |
| Trisodium EDTA | 0.12 | 0.05 | | 0.30 | |
| Ethanol | 3.00 | | 1.50 | | 5.00 |
| Perfume Oil | 0.20 | | 0.40 | 0.35 | |
| Aqua dem. | ad 100 | ad 100 | ad 100 | ad 100 | ad 100 |

Solids-stabilized emulsion
(Pickering emulsions)

| | 1 | 2 | 3 | 4 | 5 |
|---|---|---|---|---|---|
| Mineral Oil | | | 16.00 | 16.00 | |
| Octyldodecanol | 9.00 | 9.00 | 5.00 | | |
| Caprylic/Capric Triglyceride | 9.00 | 9.00 | 6.00 | | |
| C12-15 Alkyl Benzoate | | | | 5.00 | 8.00 |
| Butylene Glycol Dicaprylate/Dicaprate | | | | | 8.00 |
| Dicaprylyl Ether | 9.00 | | | 4.00 | |
| Dicaprylyl Carbonate | | 9.00 | | | |
| Hydroxyoctacosanyl Hydroxystearate | 2.00 | 2.00 | 2.20 | 2.50 | 1.50 |
| Disteardimonium Hectorite | 1.00 | 0.75 | | 0.50 | 0.25 |
| Cera Microcristallina + Paraffinum Liquidum | | 0.35 | | | 5.00 |
| Hydroxypropyl Methylcellulose | | | 0.10 | | 0.05 |
| Dimethicone | | | | | 3.00 |
| Resilin protein | 1.0 | 0.5 | 0.1 | 3.0 | 5.0 |
| Titanium Dioxide + Alumina + Simethicone + | | 3.00 | | | |
| Aqua | | | | | |
| Titanium Dioxide + Trimethoxycaprylylsilane | | 2.00 | 4.00 | 2.00 | 4.00 |
| Silica Dimethyl Silylate | 2.50 | | | 6.00 | 2.50 |
| Boron Nitride | | | 1.00 | | |
| Starch/Sodium metaphosphate Polymer | 2.00 | | | | |
| Tapioca Starch | | 0.50 | | | |
| Sodium Chloride | 5.00 | 7.00 | 8.50 | 3.00 | 4.50 |
| Glycerin | | | | 1.00 | |
| Trisodium EDTA | 1.00 | 1.00 | 1.00 | 1.00 | 1.00 |
| Vitamin E Acetate | 5.00 | 10.00 | 3.00 | 6.00 | 10.00 |
| Ascorbyl Palmitate | 1.00 | 1.00 | | 1.00 | |
| Methylparaben | | 0.60 | | | 0.20 |
| Propylparaben | | | | | 0.20 |
| Phenoxyethanol | | | 0.20 | | |
| Hexamidine Diisethionate | | | 0.40 | 0.50 | 0.40 |
| Diazolidinyl Urea | | | | | 0.08 |
| Ethanol | | | 0.23 | 0.20 | |
| Perfume Oil | 5.00 | | 3.00 | 4.00 | |
| Aqua dem. | 0.20 | | 0.30 | 0.10 | |
| | ad 100 | ad 100 | ad 100 | ad 100 | ad 100 |

Sticks

| | 1 | 2 | 3 | 4 |
|---|---|---|---|---|
| Caprylic/Capric Triglyceride | 12.00 | 10.00 | 6.00 | |
| Octyldodecanol | 7.00 | 14.00 | 8.00 | 3.00 |
| Butylene Glycol Dicaprylate/ | | | | 12.00 |
| Dicaprate | | | | |
| Pentaerythrityl Tetraisostearate | 10.00 | 6.00 | 8.00 | 7.00 |
| Polyglyceryl-3 Diisostearate | 2.50 | | | |
| Bis-Diglyceryl Polyacyladipate-2 | 9.00 | 8.00 | 10.00 | 8.00 |
| Cetearyl Alcohol | 8.00 | 11.00 | 9.00 | 7.00 |
| Myristyl Myristate | 3.50 | 3.00 | 4.00 | 3.00 |
| Beeswax | 5.00 | 5.00 | 6.00 | 6.00 |
| Copernicia Cerifera (Carnauba) | 1.50 | 2.00 | 2.00 | 1.50 |
| Wax | | | | |
| Cera Alba | 0.50 | 0.50 | 0.50 | 0.40 |
| C16-40 Alkyl Stearate | | 1.50 | 1.50 | 1.50 |
| Resilin protein | 10.0 | 1.0 | 3.0 | 0.1 |
| Uvinul ® A Plus ™ | 2.00 | 1.50 | 0.75 | 9.00 |
| Titanium Dioxide-microfine | 1.00 | | 3.00 | |
| Zinc Oxide-microfine | | 1.00 | | 0.25 |
| Vitamin E Acetate | 0.50 | 1.00 | | |
| Ascorbyl Palmitate | 0.05 | | 0.05 | |
| Buxux Chinensis (Jojoba) Oil | 2.00 | 1.00 | | 1.00 |
| Perfume Oil, BHT | 0.10 | 0.25 | | 0.35 |
| Ricinus Communis (Castor) Oil | ad 100 | ad 100 | ad 100 | ad 100 |

Self-tanning PIT emulsions

| | 1 | 2 | 3 | 4 | 5 | 6 | 7 | 8 |
|---|---|---|---|---|---|---|---|---|
| Glyceryl Monostearate SE | 0.50 | 2.00 | 3.00 | 5.00 | | 0.50 | 4.00 | |
| Glyceryl Isostearate | | | | | 3.50 | 4.00 | 2.00 | |
| Isoceteth-20 | | 0.50 | | | 2.00 | | | |
| Ceteareth-12 | | 5.00 | | 1.00 | | | 3.50 | 5.00 |
| Ceteareth-20 | | 5.00 | | 1.00 | | | | 3.50 |
| PEG-100 Stearate | | | | 2.80 | | 2.30 | 3.30 | |
| Cetyl Alcohol | 5.20 | | 1.20 | 1.00 | 1.30 | | 0.50 | 0.30 |
| Cetyl Palmitate | 2.50 | 1.20 | | 1.50 | | 0.50 | | 1.50 |
| Cetyl Dimethicone Copolyol | | | | 0.50 | | 1.00 | | |
| Polyglyceryl-2 | | | | 0.75 | 0.30 | | | |
| Resilin protein | 0.1 | 0.5 | 0.01 | 5.0 | 0.5 | 3.0 | 0.025 | 10.0 |
| Dihydroxyacetone | | | 3.00 | 5.00 | | | 4.00 | |
| Uvinul ® A Plus ™ | 2.00 | 1.50 | 0.75 | 1.00 | 2.10 | 4.50 | 5.00 | 2.10 |
| Titanium Dioxide - microfine | 1.00 | | 1.50 | | 3.50 | | 1.50 | 1.00 |
| Zinc Oxide - microfine | | 1.00 | | 0.25 | | 2.00 | | 1.50 |
| C12-15 Alkyl Benzoate | 3.50 | | | 6.35 | | | | 0.10 |
| Cocoglycerides | | 3.00 | | 3.00 | | | | 1.00 |
| Dicapryl Ether | 4.50 | | | | | | | |
| Dicaprylyl Carbonate | | 4.30 | | 3.00 | | | | 7.00 |
| Dibutyl Adipate | | | | 0.50 | | | | 0.30 |
| Phenyl Trimethicone | 2.00 | | | 3.50 | | 2.00 | | |
| Cyclomethicone | | 3.00 | | | | | | |
| C1-5 Alkyl Galactomannan | | 0.50 | | | 2.00 | | | |
| Hydrogenated Coco-Glycerides | | | | | 3.00 | 4.00 | | |
| Behenoxy Dimethicone | | | | | | 1.50 | 2.00 | |
| VP/Hexadecene Copolymer | | | | 1.00 | 1.20 | | | |
| Glycerin | 4.00 | 6.00 | 5.00 | | 8.00 | 10.00 | | |
| Vitamin E Acetate | 0.20 | 0.30 | 0.40 | | 0.30 | | | |
| Butyrospermum Parkii | | 2.00 | | 3.60 | | 2.00 | | |
| (Shea Butter) | | | | | | | | |
| Iodopropyl Butylcarbamate | 0.12 | | | | 0.20 | | | |
| DMDM Hydantoin | 0.10 | | | | 0.12 | | 0.13 | |
| Methylparaben | | 0.50 | 0.30 | | 0.35 | | | |
| Phenoxyethanol | 0.50 | 0.40 | | 1.00 | | | | |
| Ethylhexylglycerin | | 0.30 | | | 1.00 | | 0.35 | |
| Ethanol | 2.00 | | 2.00 | | | 5.00 | | |
| Trisodium EDTA | 0.40 | | 0.15 | | | 0.2 | | |
| Perfume Oil | 0.20 | | 0.20 | | 0.24 | 0.16 | 0.10 | 0.10 |
| Aqua dem. | ad | ad | ad | ad | ad | ad | ad | ad |
| | 100 | 100 | 100 | 100 | 100 | 100 | 100 | 100 |

Oil gel

| | 1 | 2 | 3 | 4 |
|---|---|---|---|---|
| Caprylic/Capric Triglyceride | 12.00 | 10.00 | 6.00 | |
| Octyldodecanol | 7.00 | 14.00 | 8.00 | 3.00 |
| Butylene Glycol | | | | 12.00 |
| Dicaprylate/Dicaprate | | | | |
| Pentaerythrityl Tetraisostearate | 10.00 | 6.00 | 8.00 | 7.00 |
| Polyglyceryl-3 Diisostearate | 2.50 | | | |
| Bis-Diglyceryl Polyacyladipate-2 | 9.00 | 8.00 | 10.00 | 8.00 |
| Myristyl Myristate | 3.50 | 3.00 | 4.00 | 3.00 |
| Quaternium-18 Bentonite | 5.00 | 5.00 | 6.00 | 6.00 |
| Propylene Carbonate | 15.00 | 20.00 | 18.00 | 19.50 |
| Resilin protein | 1.0 | 0.5 | 3.0 | 5.0 |
| Vitamin E Acetate | 0.50 | 1.00 | | |
| Ascorbyl Palmitate | 0.05 | | 0.05 | |
| Buxus Chinensis (Jojoba) Oil | 2.00 | 1.00 | | 1.00 |
| Perfume Oil, BHT | 0.10 | 0.25 | | 0.35 |
| Ricinus Communis (Castor) Oil | ad 100 | ad 100 | ad 100 | ad 100 |

### Example 35

The formulations below describe cosmetic sunscreen preparations comprising a combination of at least one inorganic pigment, preferably zinc oxide and/or titanium dioxide and organic UV-A and UV-B filters.

The formulations specified below are prepared in a customary manner known to a person skilled in the art.

The content of resilin protein prepared according to Example 1 refers to 100% active ingredient. The active ingredients according to the invention can be used either in pure form or in the form of an aqueous solution. In the case of the aqueous solution, the content of water dem. in the particular formulation must be adjusted.

| | | | |
|---|---|---|---|
| A | 7.50 | Uvinul MC 80 | Ethylhexyl Cinnamate |
| | 2.00 | Uvinul M 40 | Benzophenone-3 |
| | 0.80 | Rylo PG 11 | Polyglyceryl Dimer Soyate |
| | 1.00 | Span 60 | Sorbitan Stearate |
| | 0.50 | Vitamin E Acetate | Tocopheryl Acetate |
| | 3.00 | Dracorin 100 SE | Glyceryl Stearate, PEG-100 Stearate |
| | 1.00 | Cremophor CO 410 | PEG-40 Hydrogenated Castor Oil |
| B | 3.00 | T-Lite SF | Titanium Dioxide, Alumina Hydrate, |
| | | | Dimethicone/Methicone Copolymer |
| | 1.00 | Cetiol SB 45 | Butyrospermum Parkii (Shea Butter) |
| | 6.50 | Finsolv TN | C12-15 Alkyl Benzoate |
| C | 5.00 | Butylene glycol | Butylene Glycol |
| | 0.30 | Keltrol | Xanthan Gum |
| | 0.10 | Edeta BD | Disodium EDTA |
| | 1% | Resilin protein | |
| | 0.10 | Allantoin | Allantoin |
| | 66.20 | Water dem. | Aqua dem. |
| D | 1.00 | Sepigel 305 | Polyacrylamide, C13-14 |
| | | | Isoparaffin, Laureth-7 |
| | q.s. | | Preservative |

| | | | |
|---|---|---|---|
| A | 7.50 | Uvinul MC 80 | Ethylhexyl Cinnamate |
| | 2.00 | Uvinul M 40 | Benzophenone-3 |
| | 0.80 | Rylo PG 11 | Polyglyceryl Dimer Soyate |
| | 1.00 | Span 60 | Sorbitan Stearate |
| | 0.50 | Vitamin E Acetate | Tocopheryl Acetate |
| | 3.00 | Dracorin 100 SE | Glyceryl Stearate, PEG-100 Stearate |
| | 1.00 | Cremophor CO 410 | PEG-40 Hydrogenated Castor Oil |
| B | 3.00 | Z-COTE MAX | Zinc Oxide (and) Diphenyl Capryl |
| | | | Methicone |
| | 1.00 | Cetiol SB 45 | Butyrospermum Parkii (Shea Butter) |
| | 6.50 | Finsolv TN | C12-15 Alkyl Benzoate |
| C | 5.00 | Butylene glycol | Butylene Glycol |
| | 0.30 | Keltrol | Xanthan Gum |
| | 0.10 | Edeta BD | Disodium EDTA |
| | 1% | Resilin protein | |
| | 0.10 | Allantoin | Allantoin |
| | 66.20 | Water dem. | Aqua dem. |
| D | 2.00 | Simulgel NS | Hydroxyethyl Acrylate/Sodium |
| | | | Acryloyldimethyl Taurate Copolymer, |
| | | | Squalane, Polysorbate 60 |
| | q.s. | | Preservative |

| | | | |
|---|---|---|---|
| A | 7.50 | Uvinul MC 80 | Ethylhexyl Cinnamate |
| | 2.00 | Uvinul A Plus | Diethylamino Hydroxybenzoyl Hexyl |
| | | | Benzoate |
| | 0.80 | Rylo PG 11 | Polyglyceryl Dimer Soyate |
| | 1.00 | Span 60 | Sorbitan Stearate |
| | 0.50 | Vitamin E Acetate | Tocopheryl Acetate |
| | 3.00 | Dracorin 100 SE | Glyceryl Stearate, PEG-100 Stearate |
| | 1.00 | Cremophor CO 410 | PEG-40 Hydrogenated Castor Oil |
| B | 3.00 | Z-COTE MAX | Zinc Oxide (and) Diphenyl Capryl |
| | | | Methicone |
| | 1.00 | Cetiol SB 45 | Butyrospermum Parkii (Shea Butter) |
| | 6.50 | Finsolv TN | C12-15 Alkyl Benzoate |
| C | 5.00 | Butylene glycol | Butylene Glycol |
| | 0.30 | Keltrol | Xanthan Gum |
| | 0.10 | Edeta BD | Disodium EDTA |
| | 1% | Resilin protein | |
| | 1% | Resilin protein | |
| | 0.10 | Allantoin | Allantoin |
| | 66.20 | Water dem. | Aqua dem. |
| D | 2.00 | Simulgel NS | Hydroxyethyl Acrylate/Sodium |
| | | | Acryloyldimethyl Taurate Copolymer, |
| | | | Squalane, Polysorbate 60 |
| | q.s. | | Preservative |

| | | | |
|---|---|---|---|
| A | 4.00 | Uvinul MC 80 | Ethylhexyl Cinnamate |
| | 2.00 | Uvinul T 150 | Ethyl Hexyl Triazone |
| | 2.00 | Uvinul A Plus | Diethylamino Hydroxybenzoyl Hexyl |
| | | | Benzoate |
| | 0.80 | Rylo PG 11 | Polyglyceryl Dimer Soyate |
| | 1.00 | Span 60 | Sorbitan Stearate |
| | 0.50 | Vitamin E Acetate | Tocopheryl Acetate |
| | 3.00 | Dracorin 100 SE | Glyceryl Stearate, PEG-100 Stearate |
| | 1.00 | Cremophor CO 410 | PEG-40 Hydrogenated Castor Oil |
| B | 3.00 | Z-COTE MAX | Zinc Oxide (and) Diphenyl Capryl |
| | | | Methicone |
| | 1.00 | Cetiol SB 45 | Butyrospermum Parkii (Shea Butter) |
| | 6.50 | Finsolv TN | C12-15 Alkyl Benzoate |
| C | 5.00 | Butylene glycol | Butylene Glycol |
| | 0.30 | Keltrol | Xanthan Gum |
| | 0.10 | Edeta BD | Disodium EDTA |
| | 1% | Resilin protein | |
| | 0.10 | Allantoin | Allantoin |
| | 66.20 | Water dem. | Aqua dem. |
| D | 2.00 | Simulgel NS | Hydroxyethyl Acrylate/Sodium |
| | | | Acryloyldimethyl Taurate Copolymer, |
| | | | Squalane, Polysorbate 60 |
| | q.s. | | Preservative |

| | | | |
|---|---|---|---|
| A | 4.00 | Uvinul MC 80 | Ethylhexyl Cinnamate |
| | 2.00 | Uvinul T 150 | Ethyl Hexyl Triazone |
| | 2.00 | Uvinul A Plus | Diethylamino Hydroxybenzoyl Hexyl |
| | | | Benzoate |
| | 0.80 | Rylo PG 11 | Polyglyceryl Dimer Soyate |
| | 1.00 | Span 60 | Sorbitan Stearate |
| | 0.50 | Vitamin E Acetate | Tocopheryl Acetate |
| | 3.00 | Dracorin 100 SE | Glyceryl Stearate, PEG-100 Stearate |
| | 1.00 | Cremophor CO 410 | PEG-40 Hydrogenated Castor Oil |
| B | 3.00 | T-Lite SF | Titanium Dioxide, Alumina Hydrate, |
| | | | Dimethicone/Methicone Copolymer |
| | 1.00 | Cetiol SB 45 | Butyrospermum Parkii (Shea Butter) |
| | 6.50 | Finsolv TN | C12-15 Alkyl Benzoate |
| C | 5.00 | Butylene glycol | Butylene Glycol |
| | 0.30 | Keltrol | Xanthan Gum |
| | 0.10 | Edeta BD | Disodium EDTA |
| | 1% | Resilin protein | |
| | 0.10 | Allantoin | Allantoin |
| | 66.20 | Water dem. | Aqua dem. |
| D | 2.00 | Simulgel NS | Hydroxyethyl Acrylate/Sodium |
| | | | Acryloyldimethyl Taurate Copolymer, |
| | | | Squalane, Polysorbate 60 |
| | q.s. | | Preservative |

| | | | |
|---|---|---|---|
| A | 4.00 | Uvinul MC 80 | Ethylhexyl Cinnamate |
| | 2.00 | Uvinul T 150 | Ethyl Hexyl Triazone |
| | 2.00 | Uvinul A Plus | Diethylamino Hydroxybenzoyl Hexyl |
| | | | Benzoate |
| | 0.80 | Rylo PG 11 | Polyglyceryl Dimer Soyate |
| | 1.00 | Span 60 | Sorbitan Stearate |
| | 0.50 | Vitamin E Acetate | Tocopheryl Acetate |
| | 3.00 | Dracorin 100 SE | Glyceryl Stearate, PEG-100 Stearate |
| | 1.00 | Cremophor CO 410 | PEG-40 Hydrogenated Castor Oil |
| B | 3.00 | Z-COTE MAX | Zinc Oxide (and) Diphenyl Capryl |
| | | | Methicone |
| | 1.00 | Cetiol SB 45 | Butyrospermum Parkii (Shea Butter) |
| | 6.50 | Finsolv TN | C12-15 Alkyl Benzoate |
| C | 5.00 | Butylene glycol | Butylene Glycol |
| | 0.30 | Keltrol | Xanthan Gum |
| | 0.10 | Edeta BD | Disodium EDTA |
| | 1% | Resilin protein | |
| | 0.10 | Allantoin | Allantoin |
| | 66.20 | Water dem. | Aqua dem. |
| D | 2.00 | Simulgel NS | Hydroxyethyl Acrylate/Sodium |
| | | | Acryloyldimethyl Taurate Copolymer, |
| | | | Squalane, Polysorbate 60 |
| | q.s. | | Preservative |

| | | | |
|---|---|---|---|
| A | 4.00 | Uvinul MC 80 | Ethylhexyl Cinnamate |
| | 1.00 | Uvinul T 150 | Ethyl Hexyl Triazone |
| | 2.00 | Parsol 1789 | Butyl Methoxydibenzoylmethane |
| | 0.80 | Rylo PG 11 | Polyglyceryl Dimer Soyate |
| | 1.00 | Span 60 | Sorbitan Stearate |
| | 0.50 | Vitamin E Acetate | Tocopheryl Acetate |
| | 3.00 | Dracorin 100 SE | Glyceryl Stearate, PEG-100 Stearate |
| | 1.00 | Cremophor CO 410 | PEG-40 Hydrogenated Castor Oil |
| B | 3.00 | T-Lite SF-S | Titanium Dioxide, Silica Hydrate, |
| | | | Alumina Hydrate, |
| | | | Methicone/Dimethicone Copolymer |
| | 1.00 | Cetiol SB 45 | Butyrospermum Parkii (Shea Butter) |
| | 6.50 | Finsolv TN | C12-15 Alkyl Benzoate |
| C | 5.00 | Butylene glycol | Butylene Glycol |
| | 0.30 | Keltrol | Xanthan Gum |
| | 0.10 | Edeta BD | Disodium EDTA |
| | 1% | Resilin protein | |
| | 0.10 | Allantoin | Allantoin |
| | 66.70 | Water dem. | Aqua dem. |
| D | 2.00 | Simulgel NS | Hydroxyethyl Acrylate/Sodium |
| | | | Acryloyldimethyl Taurate Copolymer, |
| | | | Squalane, Polysorbate 60 |
| | q.s. | | Preservative |

| | | | |
|---|---|---|---|
| A | 4.00 | Uvinul MC 80 | Ethylhexyl Cinnamate |
| | 1.00 | Uvinul T 150 | Ethyl Hexyl Triazone |
| | 2.00 | Parsol 1789 | Butyl Methoxydibenzoylmethane |
| | 0.80 | Rylo PG 11 | Polyglyceryl Dimer Soyate |
| | 1.00 | Span 60 | Sorbitan Stearate |
| | 0.50 | Vitamin E Acetate | Tocopheryl Acetate |
| | 3.00 | Dracorin 100 SE | Glyceryl Stearate, PEG-100 Stearate |
| | 1.00 | Cremophor CO 410 | PEG-40 Hydrogenated Castor Oil |
| B | 3.00 | Z-COTE MAX | Zinc Oxide (and) Diphenyl Capryl |
| | | | Methicone |
| | 1.00 | Cetiol SB 45 | Butyrospermum Parkii (Shea Butter) |
| | 6.50 | Finsolv TN | C12-15 Alkyl Benzoate |
| C | 5.00 | Butylene glycol | Butylene Glycol |
| | 0.30 | Keltrol | Xanthan Gum |
| | 0.10 | Edeta BD | Disodium EDTA |
| | 1% | Resilin protein | |
| | 0.10 | Allantoin | Allantoin |
| | 67.10 | Water dem. | Aqua dem. |
| D | 2.00 | Simulgel NS | Hydroxyethyl Acrylate/Sodium |
| | | | Acryloyldimethyl Taurate Copolymer, |
| | | | Squalane, Polysorbate 60 |
| | q.s. | | Preservative |

| | | | |
|---|---|---|---|
| A | 5.00 | Uvinul N 539 T | Octocrylene |
| | 2.00 | Parsol 1789 | Butyl Methoxydibenzoylmethane |
| | 0.80 | Rylo PG 11 | Polyglyceryl Dimer Soyate |
| | 1.00 | Span 60 | Sorbitan Stearate |
| | 0.50 | Vitamin E Acetate | Tocopheryl Acetate |
| | 3.00 | Dracorin 100 SE | Glyceryl Stearate, PEG-100 Stearate |
| | 1.00 | Cremophor CO 410 | PEG-40 Hydrogenated Castor Oil |
| B | 3.00 | T-Lite SF-S | Titanium Dioxide, Silica Hydrate, |
| | | | Alumina Hydrate, |
| | | | Methicone/Dimethicone Copolymer |
| | 1.00 | Cetiol SB 45 | Butyrospermum Parkii (Shea Butter) |
| | 6.50 | Finsolv TN | C12-15 Alkyl Benzoate |
| C | 5.00 | Butylene glycol | Butylene Glycol |
| | 0.30 | Keltrol | Xanthan Gum |
| | 0.10 | Edeta BD | Disodium EDTA |
| | 0.10 | Allantoin | Allantoin |
| | 1% | Resilin protein | |
| | 66.20 | Water dem. | Aqua dem. |
| D | 2.00 | Simulgel NS | Hydroxyethyl Acrylate/Sodium |
| | | | Acryloyldimethyl Taurate Copolymer, |
| | | | Squalane, Polysorbate 60 |
| | q.s. | | Preservative |

| | | | |
|---|---|---|---|
| A | 5.00 | Uvinul N 539 T | Octocrylene |
| | 2.00 | Uvinul A Plus | Diethylamino Hydroxybenzoyl Hexyl |
| | | | Benzoate |
| | 0.80 | Rylo PG 11 | Polyglyceryl Dimer Soyate |
| | 1.00 | Span 60 | Sorbitan Stearate |
| | 0.50 | Vitamin E Acetate | Tocopheryl Acetate |
| | 3.00 | Dracorin 100 SE | Glyceryl Stearate, PEG-100 Stearate |
| | 1.00 | Cremophor CO 410 | PEG-40 Hydrogenated Castor Oil |
| B | 3.00 | T-Lite SF | Titanium Dioxide, Alumina Hydrate, |
| | | | Dimethicone/Methicone Copolymer |
| | 1.00 | Cetiol SB 45 | Butyrospermum Parkii (Shea Butter) |
| | 6.50 | Finsolv TN | C12-15 Alkyl Benzoate |
| C | 5.00 | Butylene glycol | Butylene Glycol |
| | 0.30 | Keltrol | Xanthan Gum |
| | 0.10 | Edeta BD | Disodium EDTA |
| | 2% | Resilin protein | |
| | 0.10 | Allantoin | Allantoin |
| | 65.20 | Water dem. | Aqua dem. |
| D | 2.00 | Simulgel NS | Hydroxyethyl Acrylate/Sodium |
| | | | Acryloyldimethyl Taurate Copolymer, |
| | | | Squalane, Polysorbate 60 |
| | q.s. | | Preservative |

| | | | |
|---|---|---|---|
| A | 5.00 | Uvinul N 539 T | Octocrylene |
| | 2.00 | Uvinul A Plus | Diethylamino Hydroxybenzoyl Hexyl |
| | | | Benzoate |
| | 0.80 | Rylo PG 11 | Polyglyceryl Dimer Soyate |
| | 1.00 | Span 60 | Sorbitan Stearate |
| | 0.50 | Vitamin E Acetate | Tocopheryl Acetate |
| | 3.00 | Dracorin 100 SE | Glyceryl Stearate, PEG-100 Stearate |
| | 1.00 | Cremophor CO 410 | PEG-40 Hydrogenated Castor Oil |
| B | 3.00 | Z-COTE MAX | Zinc Oxide (and) Diphenyl Capryl |
| | | | Methicone |
| | 1.00 | Cetiol SB 45 | Butyrospermum Parkii (Shea Butter) |
| | 6.50 | Finsolv TN | C12-15 Alkyl Benzoate |
| C | 5.00 | Butylene glycol | Butylene Glycol |
| | 0.30 | Keltrol | Xanthan Gum |
| | 0.10 | Edeta BD | Disodium EDTA |
| | 0.5% | Resilin protein | |
| | 0.10 | Allantoin | Allantoin |
| | 66.70 | Water dem. | Aqua dem. |
| D | 2.00 | Simulgel NS | Hydroxyethyl Acrylate/Sodium |
| | | | Acryloyldimethyl Taurate |
| | | | Copolymer, Squalane, |
| | | | Polysorbate 60 |
| | q.s. | | Preservative |

| | | | |
|---|---|---|---|
| A | 5.00 | Uvinul N 539 T | Octocrylene |
| | 2.00 | Uvinul A Plus | Diethylamino Hydroxybenzoyl Hexyl |
| | | | Benzoate |
| | 0.80 | Rylo PG 11 | Polyglyceryl Dimer Soyate |
| | 1.00 | Span 60 | Sorbitan Stearate |
| | 0.50 | Vitamin E Acetate | Tocopheryl Acetate |
| | 3.00 | Dracorin 100 SE | Glyceryl Stearate, PEG-100 Stearate |
| | 1.00 | Cremophor CO 410 | PEG-40 Hydrogenated Castor Oil |
| B | 3.00 | Z-COTE MAX | Zinc Oxide (and) Diphenyl Capryl |
| | | | Methicone |
| | 1.00 | Cetiol SB 45 | Butyrospermum Parkii (Shea Butter) |
| | 6.50 | Finsolv TN | C12-15 Alkyl Benzoate |
| C | 5.00 | Butylene glycol | Butylene Glycol |
| | 0.30 | Keltrol | Xanthan Gum |
| | 0.10 | Edeta BD | Disodium EDTA |
| | 0.10 | Allantoin | Allantoin |
| | 1% | Resilin protein | |
| | 2.0 | Mexoryl SX | Terephthalidene Dicamphor Sulfonic |
| | | | Acid |
| | 66.20 | Water dem. | Aqua dem. |
| D | 2.00 | Simulgel NS | Hydroxyethyl Acrylate/Sodium |
| | | | Acryloyldimethyl Taurate Copolymer, |
| | | | Squalane, Polysorbate 60 |
| | q.s. | | Preservative |

| | | | |
|---|---|---|---|
| A | 5.00 | Uvinul N 539 T | Octocrylene |
| | 2.00 | Uvinul A Plus | Diethylamino Hydrobenzoyl |
| | | | Hexyl Benzoate |
| | 0.80 | Rylo PG 11 | Polyglyceryl Dimer Soyate |
| | 1.00 | Span 60 | Sorbitan Stearate |
| | 0.50 | Vitamin E Acetate | Tocopheryl Acetate |
| | 3.00 | Dracorin 100 SE | Glyceryl Stearate, PEG-100 Stearate |
| | 1.00 | Cremophor CO 410 | PEG-40 Hydrogenated Castor Oil |
| B | 3.00 | Z-COTE MAX | Zinc Oxide (and) Diphenyl |
| | | | Capryl Methicone |
| | 1.00 | Cetiol SB 45 | Butyrospermum Parkii (Shea Butter) |
| | 6.50 | Finsolv TN | C12-15 Alkyl Benzoate |
| C | 5.00 | Butylene glycol | Butylene Glycol |
| | 0.30 | Keltrol | Xanthan Gum |
| | 0.10 | Edeta BD | Disodium EDTA |
| | 0.10 | Allantoin | Allantoin |
| | 2% | Resilin protein | |
| | 2.0 | Mexoryl SX | Terephthalidene Dicamphor Sulfonic |
| | | | Acid |
| | 65.20 | Water dem. | Aqua dem. |
| D | 2.00 | Simulgel NS | Hydroxyethyl Acrylate/Sodium |
| | | | Acryloyldimethyl Taurate |
| | | | Copolymer, Squalane, Polysorbate 60 |
| | q.s. | | Preservative |

| | | | |
|---|---|---|---|
| A | 5.00 | Uvinul N 539 T | Octocrylene |
| | 2.00 | Uvinul A Plus | Diethylamino Hydroxybenzoyl |
| | | | Hexyl Benzoate |
| | 2.00 | Mexoryl XL | Drometrizole Trisiloxane |
| | 0.80 | Rylo PG 11 | Polyglyceryl Dimer Soyate |
| | 1.00 | Span 60 | Sorbitan Stearate |
| | 0.50 | Vitamin E Acetate | Tocopheryl Acetate |
| | 3.00 | Dracorin 100 SE | Glyceryl Stearate, PEG-100 Stearate |
| | 1.00 | Cremophor CO 410 | PEG-40 Hydrogenated Castor Oil |
| B | 3.00 | Z-COTE MAX | Zinc Oxide(and) Diphenyl |
| | | | Capryl Methicone |
| | 1.00 | Cetiol SB 45 | Butyrospermum Parkii (Shea Butter) |
| | 6.50 | Finsolv TN | C12-15 Alkyl Benzoate |
| C | 5.00 | Butylene glycol | Butylene Glycol |
| | 0.30 | Keltrol | Xanthan Gum |
| | 0.10 | Edeta BD | Disodium EDTA |
| | 0.1% | Resilin protein | |
| | 0.10 | Allantoin | Allantoin |
| | 67.10 | Water dem. | Aqua dem. |
| D | 2.00 | Simulgel NS | Hydroxyethyl Acrylate/Sodium |
| | | | Acryloyldimethyl Taurate |
| | | | Copolymer, Squalane, Polysorbate 60 |
| | q.s. | | Preservative |

| | | | |
|---|---|---|---|
| A | 5.00 | Uvinul N 539 T | Octocrylene |
| | 2.00 | Tinosorb S | Bis-Ethylhexyloxyphenol |
| | | | Methoxyphenyl Triazine |
| | 0.80 | Rylo PG 11 | Polyglyceryl Dimer Soyate |
| | 1.00 | Span 60 | Sorbitan Stearate |
| | 0.50 | Vitamin E Acetate | Tocopheryl Acetate |
| | 3.00 | Dracorin 100 SE | Glyceryl Stearate, PEG-100 Stearate |
| | 1.00 | Cremophor CO 410 | PEG-40 Hydrogenated Castor Oil |
| B | 3.00 | Z-COTE MAX | Zinc Oxide (and) Diphenyl |
| | | | Capryl Methicone |
| | 1.00 | Cetiol SB 45 | Butyrospermum Parkii (Shea Butter) |
| | 6.50 | Finsolv TN | C12-15 Alkyl Benzoate |
| C | 5.00 | Butylene glycol | Butylene Glycol |
| | 0.30 | Keltrol | Xanthan Gum |
| | 0.10 | Edeta BD | Disodium EDTA |
| | 1% | Resilin protein | |
| | 0.10 | Allantoin | Allantoin |
| | 66.20 | Water dem. | Aqua dem. |
| D | 2.00 | Simulgel NS | Hydroxyethyl Acrylate/Sodium |
| | | | Acryloyldimethyl Taurate |
| | | | Copolymer, Squalane, Polysorbate 60 |
| | q.s. | | Preservative |

| | | | |
|---|---|---|---|
| A | 7.50 | Uvinul MC 80 | Ethylhexyl Methoxycinnamate |
| | 5.00 | Uvinul N 539 T | Octocrylene |
| | 3.00 | Emulgade PL 68/50 | Cetearyl Glucoside, Cetearyl Alcohol |
| | 2.00 | Dracorin 100 SE | Glyceryl Stearate, PEG-100 Stearate |
| | 1.00 | Fitoderm | Squalane |
| | 0.50 | Cremophor WO 7 | PEG-7 Hydrogenated Castor Oil |
| | 0.50 | Cremophor PS 20 | Polysorbate 20 |
| | 2.00 | Dry Flo Pure | Aluminum Starch Octenylsuccinate |
| B | 5.00 | Z-COTE MAX | Zinc Oxide (and) Diphenyl Capryl |
| | | | Methicone |
| C | 4.00 | 1,2-Propylene | Propylene Glycol |
| | | Glycol Care | |
| | 0.20 | Keltrol | Xanthan Gum |
| | 2% | Resilin protein | |
| | 0.50 | Simulgel 600 | Acrylamide/Sodium |
| | | | Acryloyldimethyltaurate Copolymer, |
| | | | Isohexadecane, Polysorbate 80 |
| | ad 100 | Water dem. | Aqua dem. |
| | q.s. | Preservative | |
| | 0.50 | Vitamin E Acetate | Tocopheryl Acetate |
| | 1.00 | RetiSTAR | Caprylic/Capric Triglyceride, Sodium |
| | | | Ascorbate, Tocopherol, Retinol |

| | | | |
|---|---|---|---|
| A | 7.50 | Uvinul MC 80 | Ethylhexyl Methoxycinnamate |
| | 2.00 | Uvinul N 539 T | Octocrylene |
| | 3.00 | Emulgade PL 68/50 | Cetearyl Glucoside, Cetearyl Alcohol |
| | 2.00 | Dracorin 100 SE | Glyceryl Stearate, PEG-100 Stearate |
| | 1.00 | Fitoderm | Squalane |
| | 0.50 | Cremophor WO 7 | PEG-7 Hydrogenated Castor Oil |
| | 0.50 | Cremophor PS 20 | Polysorbate 20 |
| | 2.00 | Dry Flo Pure | Aluminum Starch Octenylsuccinate |
| B | 5.00 | T-Lite SF-S | Titanium Dioxide, Silica Hydrate, |
| | | | Alumina Hydrate, Methicone/ |
| | | | Dimethicone Copolymer |
| C | 4.00 | 1,2-Propylene | Propylene Glycol |
| | | Glycol Care | |
| | 0.20 | Keltrol | Xanthan Gum |
| | 2% | Resilin protein | |
| | 0.50 | Simulgel 600 | Acrylamide/Sodium |
| | | | Acryloyldimethyltaurate Copolymer, |
| | | | Isohexadecane, Polysorbate 80 |
| | ad 100 | Water dem. | Aqua dem. |
| | q.s. | Preservative | |
| | 0.50 | Vitamin E Acetate | Tocopheryl Acetate |
| | 2.00 | RetiSTAR | Caprylic/Capric Triglyceride, Sodium |
| | | | Ascorbate, Tocopherol, Retinol |

| | | | |
|---|---|---|---|
| A | 7.50 | Uvinul MC 80 | Ethylhexyl Methoxycinnamate |
| | 5.00 | Uvinul N 539 T | Octocrylene |
| | 3.00 | Emulgade PL 68/50 | Cetearyl Glucoside, Cetearyl Alcohol |
| | 2.00 | Dracorin 100 SE | Glyceryl Stearate, PEG-100 Stearate |
| | 1.00 | Fitoderm | Squalane |
| | 0.50 | Cremophor WO 7 | PEG-7 Hydrogenated Castor Oil |
| | 0.50 | Cremophor PS 20 | Polysorbate 20 |
| | 2.00 | Dry Flo Pure | Aluminum Starch Octenylsuccinate |
| B | 5.00 | T-Lite SF | Titanium Dioxide, Alumina Hydrate, |
| | | | Dimethicone/Methicone Copolymer |
| C | 4.00 | 1,2-Propylene | Propylene Glycol |
| | | Glycol Care | |
| | 1.00 | D-Panthenol 50 P | Panthenol, Propylene Glycol |
| | 0.20 | Keltrol | Xanthan Gum |
| | 1.0% | Resilin protein | |
| | 0.50 | Simulgel 600 | Acrylamide/Sodium |
| | | | Acryloyldimethyltaurate Copolymer, |
| | | | Isohexadecane, Polysorbate 80 |
| | ad 100 | Water dem. | Aqua dem. |
| | q.s. | Preservative | |
| | 0.50 | Vitamin E Acetate | Tocopheryl Acetate |
| | 1.00 | RetiSTAR | Caprylic/Capric Triglyceride, Sodium |
| | | | Ascorbate, Tocopherol, Retinol |

| | | | |
|---|---|---|---|
| A | 5.50 | Uvinul MC 80 | Ethylhexyl Methoxycinnamate |
| | 2.00 | Uvinul A Plus | Diethylamino Hydroxybenzoyl Hexyl |
| | | | Benzoate |
| | 3.00 | Uvinul N 539 T | Octocrylene |
| | 3.00 | Emulgade PL 68/50 | Cetearyl Glucoside, Cetearyl Alcohol |
| | 2.00 | Dracorin 100 SE | Glyceryl Stearate, PEG-100 Stearate |
| | 1.00 | Fitoderm | Squalane |
| | 0.50 | Cremophor WO 7 | PEG-7 Hydrogenated Castor Oil |
| | 0.50 | Cremophor PS 20 | Polysorbate 20 |
| | 2.00 | Dry Flo Pure | Aluminum Starch Octenylsuccinate |
| B | 5.00 | T-Lite SF | Titanium Dioxide, Alumina Hydrate, |
| | | | Dimethicone/Methicone Copolymer |
| C | 4.00 | 1,2-Propylene | Propylene Glycol |
| | | Glycol Care | |
| | 0.20 | Keltrol | Xanthan Gum |
| | 2% | Resilin protein | |
| | 0.50 | Simulgel 600 | Acrylamide/Sodium |
| | | | Acryloyldimethyltaurate Copolymer, |
| | | | Isohexadecane, Polysorbate 80 |
| | Ad 100 | Water dem. | Aqua dem. |
| | q.s. | Preservative | |
| | 0.50 | Vitamin E Acetate | Tocopheryl Acetate |
| | 1.00 | RetiSTAR | Caprylic/Capric Triglyceride, Sodium |
| | | | Ascorbate, Tocopherol, Retinol |

| | | | |
|---|---|---|---|
| A | 1.00 | Abil Care 85 | Bis-PEG/PPG-16/16 |
| | | | PEG/PPG-16/16 Dimethicone, |
| | | | Caprylic/Capric Triglyceride |
| | 3.00 | Cremophor CO 40 | PEG-40 Hydrogenated Castor Oil |
| | 0.30 | Cremophor WO 7 | PEG-7 Hydrogenated Castor Oil |
| | 2.00 | Parsol 1789 | Butyl Methoxydibenzoylmethane |
| | 2.00 | Mexoryl XL | Drometrizole Trisiloxane |
| | 10.00 | Witconol APM | PPG-3 Myristyl Ether |
| | 1.00 | Uvinul T 150 | Ethylhexyl Triazone |
| | 1.00 | Dow Corning 345 Fluid | Cyclopentasiloxane, |
| | | | Cyclohexasiloxane |
| | 5.00 | Uvinul N 539 T | Octocrylene |
| B | 3.00 | Z-COTE MAX | Zinc Oxide (and) Diphenyl |
| | | | Capryl Methicone |
| C | 5.00 | 1,2-Propylene glycol | Propylene Glycol |
| | 1.00 | Mexoryl SX | Terephthalidene Dicamphor |
| | | | Sulfonic Acid |
| | 0.20 | Keltrol | Xanthan Gum |
| | 0.06 | Edeta BD | Disodium EDTA |
| | 0.04 | TEA | Triethanolamine |
| | 0.30 | Carbopol Ultrez 10P | Carbomer |
| | 63.50 | Water dem. | Aqua dem. |
| | 1% | Resilin protein | |
| D | q.s. | Perfume Oil | |
| | 0.50 | Glydant | DMDM Hydantoin |

| | | | |
|---|---|---|---|
| A | 5.50 | Uvinul MC 80 | Ethylhexyl Methoxycinnamate |
| | 2.00 | Uvinul A Plus | Diethylamino Hydroxybenzoyl Hexyl |
| | | | Benzoate |
| | 3.00 | Uvinul N 539 T | Octocrylene |
| | 3.00 | Emulgade PL 68/50 | Cetearyl Glucoside, Cetearyl Alcohol |
| | 2.00 | Dracorin 100 SE | Glyceryl Stearate, PEG-100 Stearate |
| | 1.00 | Fitoderm | Squalane |
| | 0.50 | Cremophor WO 7 | PEG-7 Hydrogenated Castor Oil |
| | 0.50 | Cremophor PS 20 | Polysorbate 20 |
| | 2.00 | Dry Flo Pure | Aluminum Starch Octenylsuccinate |
| B | 5.00 | T-Lite SF-S | Titanium Dioxide, Silica Hydrate, |
| | | | Alumina Hydrate, Methicone/ |
| | | | Dimethicone Copolymer |
| C | 4.00 | 1,2-Propylene | Propylene Glycol |
| | | Glycol Care | |
| | 0.20 | Keltrol | Xanthan Gum |
| | 0.5% | Resilin protein | |
| | 2.00 | Simulgel NS | Hydroxyethyl Acrylate/Sodium |
| | | | Acryloyldimethyl Taurate |
| | | | Copolymer, Squalane, Polysorbate 60 |
| | 66.30 | Water dem. | Aqua dem. |
| | q.s. | Preservative | |
| | 0.50 | Vitamin E Acetate | Tocopheryl Acetate |
| | 1.00 | RetiSTAR | Caprylic/Capric Triglyceride, Sodium |
| | | | Ascorbate, Tocopherol, Retinol |

| | | | |
|---|---|---|---|
| A | 2.00 | Cremophor A 6 | Ceteareth-6, Stearyl Alcohol |
| | 2.00 | Cremophor A 25 | Ceteareth-25 |
| | 3.00 | Syncrowax HRC | Tribehenin |
| | 2.00 | Lanette O | Cetearyl Alcohol |
| | 2.00 | Luvitol EHO | Cetearyl Ethylhexanoate |
| | 5.00 | Uvinul MC 80 | Ethylhexyl Methoxycinnamate |
| | 1.00 | Ganex V-220 | VP/Eicosene Copolymer |
| | 7.00 | Isopropyl palmitate | Isopropyl Palmitate |
| B | 3.00 | Z-COTE MAX | Zinc Oxide (and) Diphenyl Capryl |
| | | | Methicone |
| C | 0.20 | Keltrol | Xanthan Gum |
| | 0.5% | Resilin protein | |
| | 0.50 | Simulgel NS | Hydroxyethyl Acrylate/Sodium |
| | | | Acryloyldimethyl Taurate |
| | | | Copolymer, Squalane, Polysorbate 60 |
| | 65.10 | Water dem. | Aqua dem. |
| | 0.20 | Edeta BD | Disodium EDTA |
| | 5.00 | 1,2-Propylene | Propylene Glycol |
| | | Glycol Care | |
| D | 1.00 | Vitamin E Acetate | Tocopheryl Acetate |
| | q.s. | Preservative | |

| | | | |
|---|---|---|---|
| A | 2.00 | Cremophor A 6 | Ceteareth-6, Stearyl Alcohol |
| | 2.00 | Cremophor A 25 | Ceteareth-25 |
| | 3.00 | Syncrowax HRC | Tribehenin |
| | 2.00 | Lanette O | Cetearyl Alcohol |
| | 2.00 | Luvitol EHO | Cetearyl Ethylhexanoate |
| | 5.00 | Uvinul MC 80 | Ethylhexyl Methoxycinnamate |
| | 1.00 | Ganex V-220 | VP/Eicosene Copolymer |
| | 7.00 | Isopropyl palmitate | Isopropyl Palmitate |
| B | 3.00 | T-Lite SF | Titanium Dioxide, Alumina Hydrate, |
| | | | Dimethicone/Methicone Copolymer |
| C | 0.50 | Keltrol | Xanthan Gum |
| | 0.5% | Resilin protein | |
| | 0.50 | Simulgel NS | Hydroxyethyl Acrylate/Sodium |
| | | | Acryloyldimethyl Taurate |
| | | | Copolymer, Squalane, Polysorbate 60 |
| | 65.10 | Water dem. | Aqua dem. |
| | 0.20 | Edeta BD | Disodium EDTA |
| | 5.00 | 1,2-Propylene | Propylene Glycol |
| | | Glycol Care | |
| D | 1.00 | Vitamin E Acetate | Tocopheryl Acetate |
| | q.s. | Preservative | |

| | | | |
|---|---|---|---|
| A | 2.00 | Cremophor A 6 | Ceteareth-6, Stearyl Alcohol |
| | 2.00 | Cremophor A 25 | Ceteareth-25 |
| | 3.00 | Syncrowax HRC | Tribehenin |
| | 2.00 | Lanette O | Cetearyl Alcohol |
| | 2.00 | Luvitol EHO | Cetearyl Ethylhexanoate |
| | 5.00 | Uvinul MC 80 | Ethylhexyl Methoxycinnamate |
| | 1.00 | Ganex V-220 | VP/Eicosene Copolymer |
| | 7.00 | Isopropyl palmitate | Isopropyl Palmitate |
| B | 3.00 | T-Lite SF-S | Titanium Dioxide, Silica Hydrate, |
| | | | Alumina Hydrate, |
| | | | Methicone/Dimethicone Copolymer |
| C | 0.50 | Keltrol | Xanthan Gum |
| | 1% | Resilin protein | |
| | 0.50 | Simulgel NS | Hydroxyethyl Acrylate/Sodium |
| | | | Acryloyldimethyl Taurate |
| | | | Copolymer, Squalane, Polysorbate 60 |
| | 64.60 | Water dem. | Aqua dem. |
| | 0.20 | Edeta BD | Disodium EDTA |
| | 5.00 | 1,2-Propylene | Propylene Glycol |
| | | Glycol Care | |
| D | 1.00 | Vitamin E Acetate | Tocopheryl Acetate |
| | q.s. | Preservative | |

| | | | |
|---|---|---|---|
| A | 2.00 | Cremophor A 6 | Ceteareth-6, Stearyl Alcohol |
| | 2.00 | Cremophor A 25 | Ceteareth-25 |
| | 3.00 | Syncrowax HRC | Tribehenin |
| | 2.00 | Lanette O | Cetearyl Alcohol |
| | 2.00 | Luvitol EHO | Cetearyl Ethylhexanoate |
| | 5.00 | Uvinul MC 80 | Ethylhexyl Methoxycinnamate |
| | 1.00 | Ganex V-220 | VP/Eicosene Copolymer |
| | 7.00 | Isopropyl palmitate | Isopropyl Palmitate |
| B | 3.00 | T-Lite SF-S | Titanium Dioxide, Silica Hydrate, |
| | | | Alumina Hydrate, |
| | | | Methicone/Dimethicone Copolymer |
| C | 0.50 | Keltrol | Xanthan Gum |
| | 1% | Resilin protein | |
| | 0.50 | Simulgel NS | Hydroxyethyl Acrylate/Sodium |
| | | | Acryloyldimethyl Taurate |
| | | | Copolymer, Squalane, Polysorbate 60 |
| | 64.10 | Water dem. | Aqua dem. |
| | 2.00 | Neoheliopan AP | Disodium Phenyl Dibenzimidazole |
| | | | Tetrasulfonate |
| | 0.20 | Edeta BD | Disodium EDTA |
| | 5.00 | 1,2-Propylene | Propylene Glycol |
| | | Glycol Care | |
| D | 1.00 | Vitamin E Acetate | Tocopheryl Acetate |
| | q.s. | Preservative | |

| | | | |
|---|---|---|---|
| A | 2.00 | Cremophor A 6 | Ceteareth-6, Stearyl Alcohol |
| | 2.00 | Cremophor A 25 | Ceteareth-25 |
| | 3.00 | Syncrowax HRC | Tribehenin |
| | 2.00 | Lanette O | Cetearyl Alcohol |
| | 2.00 | Luvitol EHO | Cetearyl Ethylhexanoate |
| | 7.00 | Uvinul N 539 T | Octocrylene |
| | 1.00 | Ganex V-220 | VP/Elcosene Copolymer |
| | 7.00 | Isopropyl palmitate | Isopropyl Palmitate |
| B | 3.00 | T-Lite SF-S | Titanium Dioxide, Silica Hydrate, |
| | | | Alumina Hydrate, |
| | | | Methicone/Dimethicone Copolymer |
| C | 0.50 | Keltrol | Xanthan Gum |
| | 1% | Resilin protein | |
| | 0.50 | Simulgel NS | Hydroxyethyl Acrylate/Sodium |
| | | | Acryloyldimethyl Taurate |
| | | | Copolymer, Squalane, Polysorbate 60 |
| | 64.60 | Water dem. | Aqua dem. |
| | 1.00 | Mexoryl SX | Terephthalidene Dicamphor Sulfonic |
| | | | Acid |
| | 0.20 | Edeta BD | Disodium EDTA |
| | 5.00 | 1,2-Propylene | Propylene Glycol |
| | | Glycol Care | |
| D | 1.00 | Vitamin E Acetate | Tocopheryl Acetate |
| | q.s. | Preservative | |

| | | | |
|---|---|---|---|
| A | 2.00 | Cremophor A 6 | Ceteareth-6, Stearyl Alcohol |
| | 2.00 | Cremophor A 25 | Ceteareth-25 |
| | 3.00 | Syncrowax HRC | Tribehenin |
| | 2.00 | Lanette O | Cetearyl Alcohol |
| | 2.00 | Luvitol EHO | Cetearyl Ethylhexanoate |
| | 2.00 | Mexoryl XL | Drometrizole Trisiloxane |
| | 5.00 | Uvinul N 539 T | Octocrylene |
| | 1.00 | Ganex V-220 | VP/Eicosene Copolymer |
| | 7.00 | Isopropyl palmitate | Isopropyl Palmitate |
| B | 3.00 | T-Lite SF | Titanium Dioxide, Alumina Hydrate, |
| | | | Dimethicone/Methicone Copolymer |
| C | 0.50 | Keltrol | Xanthan Gum |
| | 0.50 | Simulgel NS | Hydroxyethyl Acrylate/Sodium |
| | | | Acryloyldimethyl Taurate |
| | | | Copolymer, Squalane, Polysorbate 60 |
| | 65.10 | Water dem. | Aqua dem. |
| | 0.20 | Edeta BD | Disodium EDTA |
| | 5.00 | 1,2-Propylene | Propylene Glycol |
| | | Glycol Care | |
| D | 1.00 | Vitamin E Acetate | Tocopheryl Acetate |
| | 0.5% | Resilin protein | |
| | q.s. | Preservative | |

| | | | |
|---|---|---|---|
| | 1.00 | Urea | Urea |
| | 2.00 | Neoheliopan AP | Disodium Phenyl Dibenzimidazole |
| | | | Tetrasulfonate |
| | 2.00 | Glycerin 87% | Glycerin |
| | 5% | Resilin protein | |
| | 1.20 | Aristoflex AVC | Ammonium |
| | | | Acryloyldimethyltaurate/VP |
| | | | Copolymer |
| | 71.30 | Water dem. | Aqua dem. |
| B | 3.00 | Cremophor CO 40 | PEG-40 Hydrogenated Castor Oil |
| | q.s. | Perfume Oil | |
| | 5.00 | Miglyol 812 | Caprylic/Capric Triglyceride |
| | 2.00 | Uvinul N 539 T | Octocrylene |
| | 8.00 | Z-COTE MAX | Zinc Oxide (and) Diphenyl Capryl |
| | | | Methicone |
| | q.s. | Preservative | |

| | | |
|---|---|---|
| 5.00 | Z-COTE MAX | Zinc Oxide (and) Diphenyl Capryl |
| | | Methicone |
| 2.00 | Parsol 1789 | Butyl Methoxydibenzoylmethane |
| 1.00 | Uvinul T150 | Ethylhexyl Triazone |
| 5.00 | Uvinul MC 80 | Ethylhexyl Methoxycinnamate |
| 2.00 | Uvinul T 539 N | Octocrylene |
| 0.50 | Abil 350 | Dimethicone |
| 2.75 | Carnico wax LT 20 | Carnauba (Copernica Cerifera) Wax, |
| | | Paraffin |
| 3.70 | Candelilla wax LT 281 LJ | Candelilla (Euphorbia Cerifera) Wax |
| 1.80 | Beeswax 3050 PH | Bees Wax |
| 3.20 | TeCero wax 30445 | Microcrystalline Wax |
| 3.20 | TeCero wax 1030 K | Microcrystalline Wax |
| 1.34 | Cutina CP | Cetyl Palmitate |
| 6.40 | Vaseline | Petrolatum |
| 7.30 | Softisan 100 | Hydrogenated Coco-Glycerides |
| 10.00 | Luvitol EHO | Cetearyl Ethylhexanoate |
| 0.17 | Bisabolol nat. | Bisabolol |
| 1.84 | Vitamin E Acetate | Tocopheryl Acetate |
| 0.42 | D,L-Alpha-Tocopherol | Tocopherol |
| 1% | Resilin protein | |
| 40.38 | Castor oil | Castor (Ricinus Communis) Oil |

| | | |
|---|---|---|
| 5.00 | T-Lite SF | Titanium Dioxide, Alumina Hydrate, |
| | | Dimethicone/Methicone Copolymer |
| 2.00 | Parsol 1789 | Butyl Methoxydibenzoylmethane |
| 1.00 | Uvinul T150 | Ethylhexyl Triazone |
| 3.00 | Uvinul MC 80 | Ethylhexyl Methoxycinnamate |
| 2.00 | Uvinul N 539 T | Octocrylene |
| 0.50 | Abil 350 | Dimethicone |
| 2.75 | Carnico wax LT 20 | Carnauba (Copernica Cerifera) Wax, |
| | | Paraffin |
| 3.70 | Candelilla wax LT 281 LJ | Candelilla (Euphorbia Cerifera) Wax |
| 1.80 | Beeswax 3050 PH | Bees Wax |
| 3.20 | TeCero wax 30445 | Microcrystalline Wax |
| 3.20 | TeCero wax 1030 K | Microcrystalline Wax |
| 1.34 | Cutina CP | Cetyl Palmitate |
| 6.40 | Vaseline | Petrolatum |
| 7.30 | Softisan 100 | Hydrogenated Coco-Glycerides |
| 10.00 | Luvitol EHO | Cetearyl Ethylhexanoate |
| 0.17 | Bisabolol nat. | Bisabolol |
| 1.84 | Vitamin E Acetate | Tocopheryl Acetate |
| 0.42 | D,L-Alpha-Tocopherol | Tocopherol |
| 1% | Resilin protein | |
| 40.38 | Castor oil | Castor (Ricinus Communis) Oil |

| | | |
|---|---|---|
| 5.00 | T-Lite SF-S | Titanium Dioxide, Silica Hydrate, |
| | | Alumina Hydrate, |
| | | Methicone/Dimethicone Copolymer |
| 2.00 | Uvinul A Plus | Diethylamino Hydroxybenzoyl Hexyl |
| | | Benzoate |
| 2.00 | Mexoryl XL | Drometrizole Trisiloxane |
| 3.00 | Uvinul MC 80 | Ethylhexyl Methoxycinnamate |
| 0.50 | Abil 350 | Dimethicone |
| 2.75 | Carnico wax LT 20 | Carnauba (Copernica Cerifera) Wax, |
| | | Paraffin |
| 3.70 | Candelilla wax LT 281 LJ | Candelilla (Euphorbia Cerifera) Wax |
| 1.80 | Beeswax 3050 PH | Bees Wax |
| 3.20 | TeCero wax 30445 | Microcrystalline Wax |
| 3.20 | TeCero wax 1030 K | Microcrystalline Wax |
| 1.34 | Cutina CP | Cetyl Palmitate |
| 6.40 | Vaseline | Petrolatum |
| 7.30 | Softisan 100 | Hydrogenated Coco-Glycerides |
| 10.00 | Luvitol EHO | Cetearyl Ethylhexanoate |
| 0.17 | Bisabolol nat. | Bisabolol |
| 1.84 | Vitamin E Acetate | Tocopheryl Acetate |
| 0.42 | D,L-Alpha-Tocopherol | Tocopherol |
| 2% | Resilin protein | |
| 39.38 | Castor oil | Castor (Ricinus Communis) Oil |

| | | | |
|---|---|---|---|
| A | 6.00 | Cremophor WO 7 | PEG-7 Hydrogenated Castor Oil |
| | 2.00 | Elfacos ST 9 | PEG-45/Dodecyl Glycol Copolymer |
| | 3.00 | Isopropyl myristate | Isopropyl Myristate |
| | 8.00 | Jojoba oil | Simmondsia Chinensis (Jojoba) |
| | | | Seed Oil |
| | 4.00 | Uvinul MC 80 | Ethylhexyl Methoxycinnamate |
| | 2.00 | Uvinul A Plus | Diethylamino Hydroxybenzoyl Hexyl |
| | | | Benzoate |
| | 1.00 | Abil 350 | Dimethicone |
| B | 5.00 | Z-COTE MAX | Zinc Oxide (and) Diphenyl Capryl |
| | | | Methicone |
| | 3.00 | T-Lite SF | Titanium Dioxide, Alumina Hydrate, |
| | | | Dimethicone/Methicone Copolymer |
| C | 0.20 | Edeta BD | Disodium EDTA |
| | 5.00 | Glycerin 87% | Glycerin |
| | 1% | Resilin protein | |
| | 0.30 | Chemag 2000 | Imidazolidinyl Urea |
| | 59.00 | Water dem. | Aqua dem. |
| D | q.s. | Perfume Oil | |
| | q.s. | Preservative | |

| | | | |
|---|---|---|---|
| | 2.00 | Cremophor A 6 | Ceteareth-6, Stearyl Alcohol |
| | 2.00 | Cremophor A 25 | Ceteareth-25 |
| | 3.00 | Syncrowax HRC | Tribehenin |
| | 2.00 | Lanette O | Cetearyl Alcohol |
| | 2.00 | Luvitol EHO | Cetearyl Ethylhexanoate |
| | 5.00 | Uvinul MC 80 | Ethylhexyl Methoxycinnamate |
| | 1.00 | Uvinul T 150 | Ethylhexyl Triazone |
| | 1.00 | Ganex V-220 | VP/Eicosene Copolymer |
| | 7.00 | Isopropyl myristate | Isopropyl Myristate |
| B | 5.00 | Z-COTE MAX | Zinc Oxide (and) Diphenyl Capryl |
| | | | Methicone |
| C | 0.20 | Keltrol | Xanthan Gum |
| | 1.5% | Resilin protein | |
| | 0.50 | Simulgel NS | Hydroxyethyl Acrylate/Sodium |
| | | | Acryloyldimethyl Taurate |
| | | | Copolymer, |
| | | | Squalane, Polysorbate 60 |
| | 61.10 | Water dem. | Aqua dem. |
| | 0.20 | Edeta BD | Disodium EDTA |
| | 5.00 | 1,2-Propylene | Propylene Glycol |
| | | Glycol Care | |
| D | 1.00 | Vitamin E Acetate | Tocopheryl Acetate |
| | q.s. | Preservative | |

| | | | |
|---|---|---|---|
| A | 2.00 | Luvitol EHO | Cetearyl Ethylhexanoate |
| | 5.00 | Paraffin oil | Mineral Oil |
| | 5.00 | Z-COTE MAX | Zinc Oxide (and) |
| | | | Diphenyl Capryl |
| | | | Methicone |
| B | 1.00 | Cremophor A 6 | Ceteareth-6, Stearyl Alcohol |
| | 2.00 | Cremophor A 25 | Ceteareth-25 |
| | 3.00 | Glycerin monostearate | Glyceryl Stearate |
| | 4.00 | Cetylstearyl alcohol | Cetearyl Alcohol |
| C | 5.00 | 1,2-Propylene | Propylene Glycol |
| | | Glycol Care | |
| | 7.50 | Luviquat Care | Polyquaternium-44 |
| | q.s. | Preservative | |
| | q.s. | Perfume Oil | |
| | 1% | Resilin protein | |
| | 64.50 | Water dem. | Aqua dem. |

| | | |
|---|---|---|
| 5.00 | T-Lite SF | Titanium Dioxide, Alumina Hydrate, |
| | | Dimethicone/Methicone Copolymer |
| 10.00 | Z-COTE MAX | Zinc Oxide (and) Diphenyl Capryl |
| | | Methicone |
| 12.00 | Beeswax 3044 PH | Bees Wax |
| 8.00 | Candelilla wax LT 281 LJ | Candelilla (Euphorbia Cerifera) Wax |
| 5.00 | Tegin | Glyceryl Stearate SE |
| 5.00 | Dow Corning 345 Fluid | Cyclopentasiloxane, |
| | | Cyclohexasiloxane |
| 5.00 | Witconol APM | PPG-3 Myristyl Ether |
| 5.00 | Softisan 154 | Hydrogenated Palm Oil |
| 8.00 | Paraffin oil, high viscosity | Mineral Oil |
| 3.00 | Vaseline | Petrolatum |
| 2% | Resilin protein | |
| 32.00 | Castor oil | Castor (Ricinus Communis) Oil |
| 3.00 | Uvinul MC 80 | Ethylhexyl Methoxycinnamate |
| 2.00 | Uvinul T 150 | Ethylhexyl Triazone |
| 2.00 | Uvinul A Plus | Diethylamino Hydroxybenzoyl Hexyl |
| | | Benzoate |
| 10.00 | Z-COTE MAX | Zinc Oxide (and) Diphenyl Capryl |
| | | Methicone |
| 12.00 | Beeswax 3044 PH | Bees Wax |
| 3.00 | Vaseline | Petrolatum |
| 8.00 | Candelilla wax LT 281 LJ | Candelilla (Euphorbia Cerifera) Wax |
| 8.00 | Paraffin oil, high viscosity | Mineral Oil |
| 5.00 | Tegin | Glyceryl Stearate SE |
| 5.00 | Softisan 154 | Hydrogenated Palm Oil |
| 5.00 | Witconol APM | PPG-3 Myristyl Ether |
| 5.00 | Dow Corning 345 Fluid | Cyclopentasiloxane, |
| | | Cyclohexasiloxane |
| 0.1% | Resilin protein | |
| 28.90 | Castor oil | Castor (Ricinus Communis) Oil |

| | | | |
|---|---|---|---|
| | 5.00 | T-Lite SF | Titanium Dioxide, Alumina Hydrate, |
| | | | Dimethlcone/Methicone Copolymer |
| | 6.00 | Finsolv TN | C12-15 Alkyl Benzoate |
| | 10.00 | Uvinul MC 80 | Ethylhexyl Methoxycinnamate |
| | 6.00 | Miglyol 812 | Caprylic/Capric Triglyceride |
| | 5.00 | Arlacel P 135 | PEG-30 Dipolyhydroxystearate |
| | 2.00 | Ganex V 216 | PVP/Hexadecene Copolymer |
| | 2.00 | Elfacos ST 9 | PEG-45/Dodecyl Glycol Copolymer |
| B | 3.00 | 1,2-Propylene | Propylene Glycol |
| | | Glycol Care | |
| | 0.10 | Edeta BD | Disodium EDTA |
| | 1.00 | Magnesium sulfate | Magnesium Sulfate |
| | | 7-hydrate | |
| | 59.40 | Water dem. | Aqua dem. |
| | 0.5% | Resilin protein | |
| | q.s. | Preservative | |

| | | | |
|---|---|---|---|
| A | 4.00 | Dehymuls SBL | Polyglyceryl-2 |
| | | | Dipolyhydroxystearate, |
| | | | Dicaprylyl Ether, Cocoglycerides, |
| | | | Sorbitan Sesquioleate, Cera Alba, |
| | | | Aluminum Stearates, Dicocoyl |
| | | | Pentaerythrityl Distearyl Citrate |
| | 1.00 | Dehymuls PGPH | Polyglyceryl-2 Dipolyhydroxystearate |
| | 8.00 | Finsolv TN | C12-15 Alkyl Benzoate |
| | 4.00 | Miglyol 812 | Caprylic/Capric Triglyceride |
| | 4.00 | Uvinul MC 80 | Ethylhexyl Methoxycinnamate |
| | 2.00 | Uvinul N 539 T | Octocrylene |
| B | 5.00 | Z-COTE MAX | Zinc Oxide (and) Diphenyl Capryl |
| | | | Methicone |
| C | 3.00 | 1,2-Propylene | Propylene Glycol |
| | | Glycol Care | |
| | 0.30 | Abiol | Imidazolidinyl Urea |
| | 1.00 | Magnesium sulfate | Magnesium Sulfate |
| | | 7-hydrate | |
| | ad 100 | Water dem. | Aqua dem. |
| | 1.5% | Resilin protein | |
| D | q.s. | Preservative | |

| | | | |
|---|---|---|---|
| A | 4.00 | Dehymuls SBL | Polyglyceryl-2 |
| | | | Dipolyhydroxystearate, |
| | | | Dicaprylyl Ether, Cocoglycerides, |
| | | | Sorbitan Sesquioleate, Cera Alba, |
| | | | Aluminum Stearates, Dicocoyl |
| | | | Pentaerythrityl Distearyl Citrate |
| | 1.00 | Dehymuls PGPH | Polyglyceryl-2 Dipolyhydroxystearate |
| | 8.00 | Finsolv TN | C12-15 Alkyl Benzoate |
| | 4.00 | Miglyol 812 | Caprylic/Capric Triglyceride |
| | 4.00 | Uvinul MC 80 | Ethylhexyl Methoxycinnamate |
| | 2.00 | Uvinul N 539 T | Octocrylene |
| B | 5.00 | T-Lite SF-S | Titanium Dioxide, Silica Hydrate, |
| | | | Alumina Hydrate, |
| | | | Methicone/Dimethicone Copolymer |
| C | 3.00 | 1,2-Propylene | Propylene Glycol |
| | | Glycol Care | |
| | 0.30 | Abiol | Imidazolidinyl Urea |
| | 1.00 | Magnesium sulfate | Magnesium Sulfate |
| | | 7-hydrate | |
| | 1% | Resilin protein | |
| | ad 100 | Water dem. | Aqua dem. |
| D | q.s. | Preservative | |

| | | | |
|---|---|---|---|
| A | 4.00 | Dehymuls SBL | Polyglyceryl-2 |
| | | | Dipolyhydroxystearate, |
| | | | Dicaprylyl Ether, Cocoglycerides, |
| | | | Sorbitan Sesquioleate, Cera Alba, |
| | | | Aluminum Stearates, Dicocoyl |
| | | | Pentaerythrityl Distearyl Citrate |
| | 1.00 | Dehymuls PGPH | Polyglyceryl-2 Dipolyhydroxystearate |
| | 8.00 | Finsolv TN | C12-15 Alkyl Benzoate |
| | 4.00 | Miglyol 812 | Caprylic/Capric Triglyceride |
| | 8.00 | Uvinul A Plus B | Ethylhexyl Methoxycinnamate and |
| | | | Diethylamino Hydroxybenzoyl hexyl |
| | | | Benzoate |
| | 2.00 | Uvinul N 539 T | Octocrylene |
| B | 5.00 | Z-COTE MAX | Zinc Oxide (and) Diphenyl Capryl |
| | | | Methicone |
| C | 3.00 | 1,2-Propylene | Propylene Glycol |
| | | Glycol Care | |
| | 0.30 | Abiol | Imidazolidinyl Urea |
| | 1.00 | Magnesium sulfate | Magnesium Sulfate |
| | | 7-hydrate | |
| | 0.1% | Resilin protein | |
| | ad 100 | Water dem. | Aqua dem. |
| | q.s. | Preservative | |

| | | | |
|---|---|---|---|
| A | 4.00 | Dehymuls SBL | Polyglyceryl-2 |
| | | | Dipolyhydroxystearate, |
| | | | Dicaprylyl Ether, Cocoglycerides, |
| | | | Sorbitan Sesquioleate, Cera Alba, |
| | | | Aluminum Stearates, Dicocoyl |
| | | | Pentaerythrityl Distearyl Citrate |
| | 1.00 | Dehymuls PGPH | Polyglyceryl-2 Dipolyhydroxystearate |
| | 8.00 | Finsolv TN | C12-15 Alkyl Benzoate |
| | 4.00 | Miglyol 812 | Caprylic/Capric Triglyceride |
| | 8.00 | Uvinul A Plus B | Ethylhexyl Methoxycinnamate and |
| | | | Diethylamino Hydroxybenzoyl Hexyl |
| | | | Benzoate |
| | 2.00 | Uvinul N 539 T | Octocrylene |
| B | 5.00 | T-Lite SF | Titanium Dioxide, Alumina Hydrate, |
| | | | Dimethicone/Methicone Copolymer |
| C | 3.00 | 1,2-Propylene | Propylene Glycol |
| | | Glycol Care | |
| | 0.30 | Abiol | Imidazolidinyl Urea |
| | 1.00 | Magnesium sulfate | Magnesium Sulfate |
| | | 7-hydrate | |
| | ad 100 | Water dem. | Aqua dem. |
| | 0.2% | Resilin protein | |
| | q.s. | Preservative | |

| | | | |
|---|---|---|---|
| A | 4.00 | Dehymuls SBL | Polyglyceryl-2 |
| | | | Dipolyhydroxystearate, |
| | | | Dicaprylyl Ether, Cocoglycerides, |
| | | | Sorbitan Sesquioleate, Cera Alba, |
| | | | Aluminum Stearates, Dicocoyl |
| | | | Pentaerythrityl Distearyl Citrate |
| | 1.00 | Dehymuls PGPH | Polyglyceryl-2 Dipolyhydroxystearate |
| | 8.00 | Finsolv TN | C12-15 Alkyl Benzoate |
| | 4.00 | Miglyol 812 | Caprylic/Capric Triglyceride |
| | 8.00 | Uvinul A Plus B | Ethylhexyl Methoxycinnamate and |
| | | | Diethylamino Hydroxybenzoyl hexyl |
| | | | Benzoate |
| | 2.00 | Uvinul N 539 T | Octocrylene |
| B | 5.00 | T-Lite SF-S | Titanium Dioxide, Silica Hydrate, |
| | | | Alumina Hydrate, |
| | | | Methicone/Dimethicone Copolymer |
| C | 3.00 | 1,2-Propylene | Propylene Glycol |
| | | Glycol Care | |
| | 0.30 | Abiol | Imidazolidinyl Urea |
| | 1.00 | Magnesium sulfate | Magnesium Sulfate |
| | | 7-hydrate | |
| | 0.5% | Resilin protein | |
| | ad 100 | Water dem. | Aqua dem. |
| | q.s. | Preservative | |

| | | | |
|---|---|---|---|
| A | 4.00 | Dehymuls SBL | Polyglyceryl-2 |
| | | | Dipolyhydroxystearate, |
| | | | Dicaprylyl Ether, Cocoglycerides, |
| | | | Sorbitan Sesquioleate, Cera Alba, |
| | | | Aluminum Stearates, Dicocoyl |
| | | | Pentaerythrityl Distearyl Citrate |
| | 1.00 | Dehymuls PGPH | Polyglyceryl-2 Dipolyhydroxystearate |
| | 8.00 | Finsolv TN | C12-15 Alkyl Benzoate |
| | 4.00 | Miglyol 812 | Caprylic/Capric Triglyceride |
| | 7.00 | Uvinul MC 80 | Ethylhexyl Methoxycinnamate |
| | 2.00 | Mexoryl XL | Drometrizole Trisiloxane |
| B | 5.00 | Z-COTE MAX | Titanium Dioxide, Alumina Hydrate, |
| | | | Dimethicone/Methicone Copolymer |
| C | 3.00 | 1,2-Propylene | Propylene Glycol |
| | | Glycol Care | |
| | 0.30 | Abiol | Imidazolidinyl Urea |
| | 1.00 | Magnesium sulfate | Magnesium Sulfate |
| | | 7-hydrate | |
| | ad 100 | Water dem. | Aqua dem. |
| | 0.01% | Resilin protein | |
| | q.s. | Preservative | |

| | | | |
|---|---|---|---|
| A | 7.50 | Uvinul MC 80 | Ethylhexyl Methoxycinnamate |
| | 3.00 | Uvinul N 539 T | Octocrylene |
| | 2.00 | Uvinul A Plus | Diethylamino Hydroxybenzoyl Hexyl |
| | | | Benzoate |
| | 1.00 | Cremophor CO 40 | PEG-40 Hydrogenated Castor Oil |
| | 10.00 | Miglyol 812 | Caprylic/Capric Triglyceride |
| | 1.50 | Dow Corning | Cyclopentasiloxane, |
| | | 345 Fluid | Cyclohexasiloxane |
| | 0.05% | Resilin protein | |
| B | 3.50 | Luvigel EM | Caprylic/Capric Triglyceride, Sodium |
| | | | Acrylates Copolymer |
| C | 46.00 | Water dem. | Aqua dem. |
| D | 5.00 | 1,2-Propylene | Propylene Glycol |
| | | Glycol Care | |
| | 0.50 | Cremophor A 25 | Ceteareth-25 |
| | 20.00 | Ethanol 96% | Alcohol |

| | | | |
|---|---|---|---|
| A | 1.00 | Uvinul A Plus | Diethylamino Hydroxybenzoyl Hexyl |
| | | | Benzoate |
| | 1.00 | Tinosorb S | Bis-Ethylhexyloxyphenol |
| | | | Methoxyphenyl Triazine |
| | 3.00 | Uvinul MC 80 | Ethylhexyl Methoxycinnamate |
| | 8.00 | Miglyol 812 | Caprylic/Capric Triglyceride |
| | 1.50 | Dow Corning | Dimethicone |
| | | 350 Fluid | |
| | 3.00 | Z-COTE MAX | Titanium Dioxide, Alumina Hydrate, |
| | | | Dimethicone/Methicone Copolymer |
| | 3.00 | Finsolv TN | C12-15 Alkyl Benzoate |
| | 1.00 | Cremophor CO 40 | PEG-40 Hydrogenated Castor Oil |
| B | 2.00 | Luvigel EM | Caprylic/Capric Triglyceride, Sodium |
| | | | Acrylates Copolymer |
| C | 54.80 | Water dem. | Aqua dem. |
| D | 15.00 | Ethanol 96% | Alcohol |
| | 5.00 | 1,2-Propylene | Propylene Glycol |
| | | Glycol Care | |
| | 0.50 | Cremophor A 25 | Ceteareth-25 |
| | 1% | Resilin protein | |
| | 1.00 | Vitamin E Acetate | Tocopheryl Acetate |
| | 0.20 | Bisabolol rac. | Bisabolol |

| | | | |
|---|---|---|---|
| A | 4.00 | Dehymuls SBL | Polyglyceryl-2 |
| | | | Dipolyhydroxystearate, Dicaprylyl |
| | | | Ether, Cocoglycerides, Sorbitan |
| | | | Sesquioleate, Cera Alba, Aluminum |
| | | | Stearates, Dicocoyl Pentaerythrityl |
| | | | Distearyl Citrate |
| | 1.00 | Dehymuls PGPH | Polyglyceryl-2 |
| | | | Dipolyhydroxystearate |
| | 6.00 | Finsolv TN | C12-15 Alkyl Benzoate |
| | 6.00 | Miglyol 812 | Caprylic/Capric Triglyceride |
| | 5.00 | Uvinul MC 80 | Ethylhexyl Methoxycinnamate |
| | 3.00 | Neoheliopan HMS | Homosalate |
| B | 5.00 | Z-COTE MAX | Zinc Oxide (and) Diphenyl Capryl |
| | | | Methicone |
| C | 3.00 | 1,2-Propylene | Propylene Glycol |
| | | Glycol Care | |
| | 0.30 | Chemag 2000 | Imidazolidinyl Urea |
| | 0.1% | Resilin protein | |
| | 1.00 | Magnesium sulfate | Magnesium Sulfate |
| | | 7-hydrate | |
| | 65.10 | Water dem. | Aqua dem. |
| | q.s. | Preservative | |

| | | | |
|---|---|---|---|
| A | 4.00 | Tego Care 450 | Polyglyceryl-3 Methyl Glucose |
| | | | Distearate |
| | 4.50 | Uvinul MC 80 | Ethylhexyl Methoxycinnamate |
| | 3.00 | Uvinul N 539 T | Octocrylene |
| | 2.00 | Uvinul T 150 | Ethylhexyl Triazone |
| | 0.50 | Vitamin E Acetate | Tocopheryl Acetate |
| B | 5.00 | Isohexadecane | Isohexadecane |
| | 5.00 | Cosmacol EMI | Di-C12-13 Alkyl Malate |
| | 3.50 | Cetiol SN | Cetearyl Isononanoate |
| | 1.00 | Ganex V-220 | VP/Eicosene Copolymer |
| | 0.2% | Resilin protein | |
| | 5.00 | T-Lite SF | Titanium Dioxide, Alumina Hydrate, |
| | | | Dimethicone/Methicone Copolymer |
| C | 60.00 | Water dem. | Water |
| | 5.00 | Glycerin 87% | Glycerin |
| | 1.00 | Lanette E | Sodium Cetearyl Sulfate |
| | 0.30 | Keltrol | Xanthan Gum |
| D | q.s. | Preservative | |

| | | | |
|---|---|---|---|
| A | 4.00 | Tego Care 450 | Polyglyceryl-3 Methyl Glucose |
| | | | Distearate |
| | 4.50 | Uvinul MC 80 | Ethylhexyl Methoxycinnamate |
| | 3.00 | Uvinul N 539 T | Octocrylene |
| | 2.00 | Neoheliopan HMS | Homosalate |
| | 0.50 | Vitamin E Acetate | Tocopheryl Acetate |
| B | 5.00 | Isohexadecane | Isohexadecane |
| | 5.00 | Cosmacol EMI | Di-C12-13 Alkyl Malate |
| | 3.50 | Cetiol SN | Cetearyl Isononanoate |
| | 1.00 | Ganex V-220 | VP/Eicosene Copolymer |
| | 1.0% | Resilin protein | |
| | 5.00 | T-Lite SF | Titanium Dioxide, Alumina Hydrate, |
| | | | Dimethicone/Methicone Copolymer |
| C | 59.20 | Water dem. | Aqua dem. |
| | 5.00 | Glycerin 87% | Glycerin |
| | 1.00 | Lanette E | Sodium Cetearyl Sulfate |
| | 0.30 | Keltrol | Xanthan Gum |
| D | q.s. | Preservative | |

| | | | |
|---|---|---|---|
| A | 4.00 | Tego Care 450 | Polyglyceryl-3 Methyl Glucose |
| | | | Distearate |
| | 4.50 | Uvinul MC 80 | Ethylhexyl Methoxycinnamate |
| | 3.00 | Neoheliopan OS | Octisalate |
| | 2.00 | Neoheliopan HMS | Homosalate |
| | 0.50 | Vitamin E Acetate | Tocopheryl Acetate |
| B | 5.00 | Isohexadecane | Isohexadecane |
| | 5.00 | Cosmacol EMI | Di-C12-13 Alkyl Malate |
| | 3.50 | Cetiol SN | Cetearyl Isononanoate |
| | 1.00 | Ganex V-220 | VP/Eicosene Copolymer |
| | 0.05% | Resilin protein | |
| | 5.00 | Z-COTE MAX | Zinc Oxide (and) Diphenyl Capryl |
| | | | Methicone |
| C | 60.15 | Water dem. | Aqua dem. |
| | 5.00 | Glycerin 87% | Glycerin |
| | 1.00 | Lanette E | Sodium Cetearyl Sulfate |
| | 0.30 | Keltrol | Xanthan Gum |
| D | q.s. | Preservative | |

| | | | |
|---|---|---|---|
| A | 4.00 | Tego Care 450 | Polyglyceryl-3 Methyl Glucose |
| | | | Distearate |
| | 4.50 | Uvinul N 539 T | Octocrylene |
| | 3.00 | Neoheliopan OS | Octisalate |
| | 2.00 | Neoheliopan HMS | Homosalate |
| | 0.50 | Vitamin E Acetate | Tocopheryl Acetate |
| B | 5.00 | Isohexadecane | Isohexadecane |
| | 5.00 | Cosmacol EMI | Di-C12-13 Alkyl Malate |
| | 3.50 | Cetiol SN | Cetearyl Isononanoate |
| | 1.00 | Ganex V-220 | VP/Eicosene Copolymer |
| | 0.01% | Resilin protein | |
| | 5.00 | Z-COTE MAX | Zinc Oxide (and) Diphenyl Capryl |
| | | | Methicone |
| C | 60.19 | Water dem. | Aqua dem. |
| | 5.00 | Glycerin 87% | Glycerin |
| | 1.00 | Lanette E | Sodium Cetearyl Sulfate |
| | 0.30 | Keltrol | Xanthan Gum |
| D | q.s. | Preservative | |

| | | | |
|---|---|---|---|
| A | 4.00 | Tego Care 450 | Polyglyceryl-3 Methyl Glucose |
| | | | Distearate |
| | 4.50 | Uvinul N 539 T | Octocrylene |
| | 3.00 | Neoheliopan OS | Octisalate |
| | 2.00 | Neoheliopan HMS | Homosalate |
| | 0.50 | Vitamin E Acetate | Tocopheryl Acetate |
| B | 5.00 | Isohexadecane | Isohexadecane |
| | 5.00 | Cosmacol EMI | Di-C12-13 Alkyl Malate |
| | 3.50 | Cetiol SN | Cetearyl Isononanoate |
| | 1.00 | Ganex V-220 | VP/Eicosene Copolymer |
| | 5.00 | T-Lite SF-S | Titanium Dioxide, Silica Hydrate, |
| | | | Alumina Hydrate, |
| | | | Methicone/Dimethicone Copolymer |
| C | 59.20 | Water dem. | Aqua dem. |
| | 5.00 | Glycerin 87% | Glycerin |
| | 1.00 | Lanette E | Sodium Cetearyl Sulfate |
| | 0.30 | Keltrol | Xanthan Gum |
| | 1.0% | Resilin protein | |
| D | q.s. | Preservative | |

| | | | |
|---|---|---|---|
| A | 8.00 | Cetiol B | Dibutyl Adipate |
| | 8.00 | Finsolv TN | C12-15 Alkyl Benzoate |
| | 12.00 | Myritol 331 | Cocoglycerides |
| | 1.00 | Lanette E | Sodium Cetearyl Sulfate |
| | 4.00 | Eumulgin VL 75 | Lauryl Glucoside, Polyglyceryl-2 |
| | | | Dipolyhydroxystearate, Glycerin |
| | 2.00 | Lanette O | Cetearyl Alcohol |
| | 3.00 | Tinosorb S | Bis-Ethylhexyloxyphenol |
| | | | Methoxyphenyl Triazine |
| | 1.00 | Vitamin E Acetate | Tocopheryl Acetate |
| B | 4.00 | Z-COTE MAX | Zinc Oxide (and) Diphenyl Capryl |
| | | | Methicone |
| C | 3.00 | Glycerin 87% | Glycerin |
| | 0.05 | Edeta BD | Disodium EDTA |
| | 0.20 | Allantoin | Allantoin |
| | 0.30 | Keltrol | Xanthan Gum |
| | 2% | Resilin protein | |
| | 1.50 | Veegum Ultra | Magnesium Aluminum Silicate |
| | 49.45 | Water dem. | Aqua dem. |
| D | q.s. | Preservative | |

| | | | |
|---|---|---|---|
| A | 8.00 | Cetiol B | Dibutyl Adipate |
| | 8.00 | Finsolv TN | C12-15 Alkyl Benzoate |
| | 12.00 | Myritol 331 | Cocoglycerides |
| | 1.00 | Lanette E | Sodium Cetearyl Sulfate |
| | 4.00 | Eumulgin VL 75 | Lauryl Glucoside, Polyglyceryl-2 |
| | | | Dipolyhydroxystearate, Glycerin |
| | 2.00 | Lanette O | Cetearyl Alcohol |
| | 3.00 | Tinosorb S | Bis-Ethylhexyloxyphenol |
| | | | Methoxyphenyl Triazine |
| | 1.00 | Vitamin E Acetate | Tocopheryl Acetate |
| B | 4.00 | T-Lite SF | Titanium Dioxide, Alumina Hydrate, |
| | | | Dimethicone/Methicone Copolymer |
| C | 3.00 | Glycerin 87% | Glycerin |
| | 0.05 | Edeta BD | Disodium EDTA |
| | 0.20 | Allantoin | Allantoin |
| | 0.30 | Keltrol | Xanthan Gum |
| | 0.2% | Resilin protein | |
| | 1.50 | Veegum Ultra | Magnesium Aluminum Silicate |
| | 50.25 | Water dem. | Aqua dem. |
| D | 0.50 | Citric acid | Citric Acid |
| | q.s. | Perfume Oil | |
| | q.s. | Preservative | |

| | | | |
|---|---|---|---|
| A | 5.00 | Cosmacol EMI | Di-C12-13 Alkyl Malate |
| | 4.50 | Uvinul MC 80 | Ethylhexyl Methoxycinnamate |
| | 3.00 | Uvinul N 539 T | Octocrylene |
| | 2.00 | Uvinul T 150 | Ethylhexyl Triazone |
| B | 4.00 | Tego Care 450 | Polyglyceryl-3 Methyl Glucose |
| | | | Distearate |
| | 5.00 | Isohexadecane | Isohexadecane |
| | 3.50 | Cetiol SN | Cetearyl Isononanoate |
| | 0.50 | Vitamin E Acetate | Tocopheryl Acetate |
| | 1.00 | Ganex V-220 | VP/Eicosene Copolymer |
| | 2.50 | T-Lite SF | Titanium Dioxide, Alumina Hydrate, |
| | | | Dimethicone/Methicone Copolymer |
| C | 5.00 | Glycerin 87% | Glycerin |
| | 2.00 | Lanette E | Sodium Cetearyl Sulfate |
| | 0.30 | Keltrol | Xanthan Gum |
| | 1.00 | Pationic 138 C | Sodium Lauroyl Lactylate |
| | 1.0% | Resilin protein | |
| | 1.00 | Pationic SSL | Sodium Stearoyl Lactylate |
| | 42.00 | Water dem. | Aqua dem. |
| D | 5.00 | Eusolex 232 | Phenylbenzimidazole Sulfonic Acid |
| | 10.00 | Water dem. | Aqua dem. |
| E | 0.70 | Sodium hydroxide | Sodium Hydroxide |
| | q.s. | Preservative | |

| | | | |
|---|---|---|---|
| A | 5.00 | Cosmacol EMI | Di-C12-13 Alkyl Malate |
| | 4.50 | Uvinul MC 80 | Ethylhexyl Methoxycinnamate |
| | 3.00 | Uvinul N 539 T | Octocrylene |
| | 2.00 | Uvinul A Plus | Diethylamino Hydrobenzoyl Hexyl |
| | | | Benzoate |
| B | 4.00 | Tego Care 450 | Polyglyceryl-3 Methyl Glucose |
| | | | Distearate |
| | 5.00 | Isohexadecane | Isohexadecane |
| | 3.50 | Cetiol SN | Cetearyl Isononanoate |
| | 0.50 | Vitamin E Acetate | Tocopheryl Acetate |
| | 1.00 | Ganex V-220 | VP/Eicosene Copolymer |
| | 2.50 | T-Lite SF | Titanium Dioxide, Alumina Hydrate, |
| | | | Dimethicone/Methicone Copolymer |
| C | 5.00 | Glycerin 87% | Glycerin |
| | 2.00 | Lanette E | Sodium Cetearyl Sulfate |
| | 0.30 | Keltrol | Xanthan Gum |
| | 1.00 | Pationic 138 C | Sodium Lauroyl Lactylate |
| | 2% | Resilin protein | |
| | 1.00 | Pationic SSL | Sodium Stearoyl Lactylate |
| | 41.00 | Water dem. | Aqua dem. |
| D | 5.00 | Eusolex 232 | Phenylbenzimidazole Sulfonic Acid |
| | 10.00 | Water dem. | Aqua dem. |
| E | 0.70 | Sodium hydroxide | Sodium Hydroxide |
| | q.s. | Preservative | |

| | | | |
|---|---|---|---|
| A | 5.00 | Cosmacol EMI | Di-C12-13 Alkyl Malate |
| | 4.50 | Uvinul MC 80 | Ethylhexyl Methoxycinnamate |
| | 3.00 | Uvinul N 539 T | Octocrylene |
| | 2.00 | Uvinul A Plus | Diethylamino Hydrobenzoyl Hexyl |
| | | | Benzoate |
| B | 4.00 | Tego Care 450 | Polyglyceryl-3 Methyl Glucose |
| | | | Distearate |
| | 5.00 | Isohexadecane | Isohexadecane |
| | 3.50 | Cetiol SN | Cetearyl Isononanoate |
| | 0.50 | Vitamin E Acetate | Tocopheryl Acetate |
| | 1.00 | Ganex V-220 | VP/Eicosene Copolymer |
| | 2.50 | Z-COTE MAX | Zinc Oxide (and) Diphenyl Capryl |
| | | | Methicone |
| C | 5.00 | Glycerin 87% | Glycerin |
| | 2.00 | Lanette E | Sodium Cetearyl Sulfate |
| | 0.30 | Keltrol | Xanthan Gum |
| | 1.00 | Pationic 138 C | Sodium Lauroyl Lactylate |
| | 0.5% | Resilin protein | |
| | 1.00 | Pationic SSL | Sodium Stearoyl Lactylate |
| | 42.50 | Water dem. | Aqua dem. |
| D | 5.00 | Eusolex 232 | Phenylbenzimidazole Sulfonic Acid |
| | 10.00 | Water dem. | Aqua dem. |
| E | 0.70 | Sodium hydroxide | Sodium Hydroxide |
| | q.s. | Preservative | |

| | | | |
|---|---|---|---|
| A | 5.00 | Cosmacol EMI | Di-C12-13 Alkyl Malate |
| | 7.50 | Uvinul N 539 T | Octocrylene |
| | 2.00 | Mexoryl XL | Drometrizole Trisiloxane |
| B | 4.00 | Tego Care 450 | Polyglyceryl-3 Methyl Glucose |
| | | | Distearate |
| | 5.00 | Isohexadecane | Isohexadecane |
| | 3.50 | Cetiol SN | Cetearyl Isononanoate |
| | 0.50 | Vitamin E Acetate | Tocopheryl Acetate |
| | 1.00 | Ganex V-220 | VP/Eicosene Copolymer |
| | 5.00 | Z-COTE MAX | Zinc Oxide (and) Diphenyl Capryl |
| | | | Methicone |
| C | 5.00 | Glycerin 87% | Glycerin |
| | 2.00 | Lanette E | Sodium Cetearyl Sulfate |
| | 0.30 | Keltrol | Xanthan Gum |
| | 1.00 | Pationic 138 C | Sodium Lauroyl Lactylate |
| | 1.0% | Resilin protein | |
| | 1.00 | Pationic SSL | Sodium Stearoyl Lactylate |
| | 40.50 | Water dem. | Aqua dem. |
| D | 5.00 | Eusolex 232 | Phenylbenzimidazole Sulfonic Acid |
| | 10.00 | Water dem. | Aqua dem. |
| E | 0.70 | Sodium hydroxide | Sodium Hydroxide |
| | q.s. | Preservative | |

| | | | |
|---|---|---|---|
| A | 5.00 | Cosmacol EMI | Di-C12-13 Alkyl Malate |
| | 5.50 | Uvinul N 539 T | Octocrylene |
| | 2.00 | Uvinul A Plus | Diethylamino Hydrobenzoyl Hexyl |
| | | | Benzoate |
| | 2.00 | Mexoryl XL | Drometrizole Trisiloxane |
| B | 4.00 | Tego Care 450 | Polyglyceryl-3 Methyl Glucose |
| | | | Distearate |
| | 5.00 | Isohexadecane | Isohexadecane |
| | 3.50 | Cetiol SN | Cetearyl Isononanoate |
| | 0.50 | Vitamin E Acetate | Tocopheryl Acetate |
| | 1.00 | Ganex V-220 | VP/Eicosene Copolymer |
| | 5.00 | Z-COTE MAX | Zinc Oxide (and) Diphenyl Capryl |
| | | | Methicone |
| C | 5.00 | Glycerin 87% | Glycerin |
| | 2.00 | Lanette E | Sodium Cetearyl Sulfate |
| | 0.30 | Keltrol | Xanthan Gum |
| | 1.00 | Pationic 138 C | Sodium Lauroyl Lactylate |
| | 1.00 | Pationic SSL | Sodium Stearoyl Lactylate |
| | 41.50 | Water dem. | Aqua dem. |
| D | 5.00 | Eusolex 232 | Phenylbenzimidazole Sulfonic Acid |
| | 0.5% | Resilin protein | |
| | 9.50 | Water dem. | Aqua dem. |
| E | 0.70 | Sodium hydroxide | Sodium Hydroxide |
| | q.s. | Preservative | |

| | | | |
|---|---|---|---|
| A | 5.00 | Cosmacol EMI | Di-C12-13 Alkyl Malate |
| | 5.50 | Uvinul N 539 T | Octocrylene |
| | 2.00 | Uvinul A Plus | Diethylamino Hydrobenzoyl Hexyl |
| | | | Benzoate |
| | 2.00 | Mexoryl XL | Drometrizole Trisiloxane |
| B | 4.00 | Tego Care 450 | Polyglyceryl-3 Methyl Glucose |
| | | | Distearate |
| | 5.00 | Isohexadecane | Isohexadecane |
| | 3.50 | Cetiol SN | Cetearyl Isononanoate |
| | 0.50 | Vitamin E Acetate | Tocopheryl Acetate |
| | 1.00 | Ganex V-220 | VP/Eicosene Copolymer |
| | 5.00 | T-Lite SF-S | Titanium Dioxide, Silica Hydrate, |
| | | | Alumina Hydrate, |
| | | | Methicone/Dimethicone Copolymer |
| C | 5.00 | Glycerin 87% | Glycerin |
| | 2.00 | Lanette E | Sodium Cetearyl Sulfate |
| | 0.30 | Keltrol | Xanthan Gum |
| | 1.00 | Pationic 138 C | Sodium Lauroyl Lactylate |
| | 1.0% | Resilin protein | |
| | 1.00 | Pationic SSL | Sodium Stearoyl Lactylate |
| | 40.50 | Water dem. | Aqua dem. |
| D | 5.00 | Eusolex 232 | Phenylbenzimidazole Sulfonic Acid |
| | 10.00 | Water dem. | Aqua dem. |
| E | 0.70 | Sodium hydroxide | Sodium Hydroxide |
| | q.s. | Preservative | |

| | | | |
|---|---|---|---|
| A | 8.00 | Cetiol B | Dibutyl Adipate |
| | 8.00 | Finsolv TN | C12-15 Alkyl Benzoate |
| | 10.00 | Myritol 331 | Cocoglycerides |
| | 1.00 | Lanette E | Sodium Cetearyl Sulfate |
| | 4.00 | Eumulgin VL 75 | Lauryl Glucoside, Polyglyceryl-2 |
| | | | Dipolyhydroxystearate, Glycerin |
| | 2.00 | Lanette O | Cetearyl Alcohol |
| | 1.00 | Tinosorb S | Bis-Ethylhexyloxyphenol |
| | | | Methoxyphenyl Triazine |
| | 1.00 | Uvinul T 150 | Ethylhexyl Triazone |
| | 1.00 | Vitamin E Acetate | Tocopheryl Acetate |
| B | 5.00 | T-Lite SF | Titanium Dioxide, Alumina Hydrate, |
| | | | Dimethicone/Methicone Copolymer |
| C | 3.00 | Glycerin 87% | Glycerin |
| | 0.05 | Edeta BD | Disodium EDTA |
| | 0.20 | Allantoin | Allantoin |
| | 0.30 | Keltrol | Xanthan Gum |
| | 2% | Resilin protein | |
| | 1.50 | Veegum Ultra | Magnesium Aluminum Silicate |
| | ad 100 | Water dem. | Aqua dem. |
| D | q.s. | Perfume Oil | |
| | q.s. | Preservative | |

| | | | |
|---|---|---|---|
| A | 8.00 | Cetiol B | Dibutyl Adipate |
| | 8.00 | Finsolv TN | C12-15 Alkyl Benzoate |
| | 10.00 | Myritol 331 | Cocoglycerides |
| | 1.00 | Lanette E | Sodium Cetearyl Sulfate |
| | 4.00 | Eumulgin VL 75 | Lauryl Glucoside, Polyglyceryl-2 |
| | | | Dipolyhydroxystearate, Glycerin |
| | 2.00 | Lanette O | Cetearyl Alcohol |
| | 1.00 | Tinosorb S | Bis-Ethylhexyloxyphenol |
| | | | Methoxyphenyl triazine |
| | 1.00 | Uvinul T 150 | Ethylhexyl Triazone |
| | 1.00 | Vitamin E Acetate | Tocopheryl Acetate |
| B | 5.00 | Z-COTE MAX | Zinc Oxide (and) Diphenyl Capryl |
| | | | Methicone |
| C | 3.00 | Glycerin 87% | Glycerin |
| | 0.05 | Edeta BD | Disodium EDTA |
| | 0.20 | Allantoin | Allantoin |
| | 0.30 | Keltrol | Xanthan Gum |
| | 1.0% | Resilin protein | |
| | 1.50 | Veegum Ultra | Magnesium Aluminum Silicate |
| | ad 100 | Water dem. | Aqua dem. |
| D | q.s. | Perfume Oil | |
| | q.s. | Preservative | |

| | | | |
|---|---|---|---|
| A | 3.00 | Glycerin 87% | Glycerin |
| | 0.20 | Edeta BD | Disodium EDTA |
| | 0.30 | Abiol | Imidazolidinyl Urea |
| | 1.00 | Plantacare 2000 | Decyl Glucoside |
| | 0.30 | Keltrol T | Xanthan Gum |
| | 57.00 | Water dem. | Aqua dem. |
| B | 5.00 | T-Lite SF-S | Titanium Dioxide, Silica Hydrate, |
| | | | Alumina Hydrate, |
| | | | Methicone/Dimethicone Copolymer |
| C | 7.50 | Uvinul MC 80 | Ethylhexyl Methoxycinnamate |
| | 2.00 | Uvinul A Plus | Diethylamino Hydroxybenzoyl |
| | | | Hexyl Benzoate |
| | 2.00 | Dow Corning 345 | Cyclopentasiloxane, |
| | | Fluid | Cyclohexasiloxane |
| | 3.50 | Cremophor A 6 | Ceteareth-6, Stearyl Alcohol |
| | 1.50 | Cremophor A 25 | Ceteareth-25 |
| | 0.50 | Beeswax 3044 PH | Bees Wax |
| | 3.00 | Lanette O | Cetearyl Alcohol |
| | 10.00 | Miglyol 812 | Caprylic/Capric Triglyceride |
| D | 1.00 | Vitamin E Acetate | Tocopheryl Acetate |
| | 2% | Resilin protein | |
| | 0.20 | Bisabolol rac. | Bisabolol |

| | | | |
|---|---|---|---|
| A | 3.00 | Glycerin 87% | Glycerin |
| | 0.20 | Edeta BD | Disodium EDTA |
| | 0.30 | Abiol | Imidazolidinyl Urea |
| | 1.00 | Plantacare 2000 | Decyl Glucoside |
| | 1.0% | Resilin protein | |
| | 0.30 | Keltrol T | Xanthan Gum |
| | 58.00 | Water dem. | Aqua dem. |
| B | 5.00 | Z-COTE MAX | Zinc Oxide (and) Diphenyl Capryl |
| | | | Methicone |
| C | 7.50 | Uvinul MC 80 | Ethylhexyl Methoxycinnamate |
| | 2.00 | Uvinul A Plus | Diethylamino Hydroxybenzoyl |
| | | | Hexyl Benzoate |
| | 2.00 | Dow Corning 345 | Cyclopentasiloxane, |
| | | Fluid | Cyclohexasiloxane |
| | 3.50 | Cremophor A 6 | Ceteareth-6, Stearyl Alcohol |
| | 1.50 | Cremophor A 25 | Ceteareth-25 |
| | 0.50 | Beeswax 3044 PH | Bees Wax |
| | 3.00 | Lanette O | Cetearyl Alcohol |
| | 10.00 | Miglyol 812 | Caprylic/Capric Triglyceride |
| D | 1.00 | Vitamin E Acetate | Tocopheryl Acetate |
| | 0.20 | Bisabolol rac. | Bisabolol |

| | | | |
|---|---|---|---|
| A | 3.00 | Glycerin 87% | Glycerin |
| | 0.20 | Edeta BD | Disodium EDTA |
| | 0.30 | Abiol | Imidazolidinyl Urea |
| | 1.00 | Plantacare 2000 | Decyl Glucoside |
| | 0.5% | Resilin protein | |
| | 0.30 | Keltrol T | Xanthan Gum |
| | 58.50 | Water dem. | Aqua dem. |
| B | 5.00 | T-Lite SF-S | Titanium Dioxide, Silica Hydrate, |
| | | | Alumina Hydrate, |
| | | | Methicone/Dimethicone Copolymer |
| C | 7.50 | Uvinul MC 80 | Ethylhexyl Methoxycinnamate |
| | 5.00 | Uvinul N 539 T | Octocrylene |
| | 2.00 | Parsol 1789 | Butyl Methoxydibenzoylmethane |
| | 2.00 | Dow Corning 345 | Cyclopentasiloxane, |
| | | Fluid | Cyclohexasiloxane |
| | 3.50 | Cremophor A 6 | Ceteareth-6, Stearyl Alcohol |
| | 1.50 | Cremophor A 25 | Ceteareth-25 |
| | 0.50 | Beeswax 3044 PH | Bees Wax |
| | 3.00 | Lanette O | Cetearyl Alcohol |
| | 10.00 | Miglyol 812 | Caprylic/Capric Triglyceride |
| D | 1.00 | Vitamin E Acetate | Tocopheryl Acetate |
| | 0.20 | Bisabolol rac. | Bisabolol |

| | | | |
|---|---|---|---|
| A | 3.00 | Glycerin 87% | Glycerin |
| | 0.20 | Edeta BD | Disodium EDTA |
| | 0.30 | Abiol | Imidazolidinyl Urea |
| | 1.00 | Plantacare 2000 | Decyl Glucoside |
| | 0.30 | Keltrol T | Xanthan Gum |
| | 2.00 | Mexoryl SL | Terephthalidene Dicamphor |
| | | | Sulfonic Acid |
| | 58.40 | Water dem. | Aqua dem. |
| B | 6.00 | T-Lite SF-S | Titanium Dioxide, Silica Hydrate, |
| | | | Alumina Hydrate, |
| | | | Methicone/Dimethicone Copolymer |
| | 6.00 | Uvinul N 539 T | Octocrylene |
| | 2.00 | Parsol 1789 | Butyl Methoxydibenzoylmethane |
| | 2.00 | Dow Corning 345 | Cyclopentasiloxane, |
| | | Fluid | Cyclohexasiloxane |
| | 3.50 | Cremophor A 6 | Ceteareth-6, Stearyl Alcohol |
| | 1.50 | Cremophor A 25 | Ceteareth-25 |
| | 0.50 | Beeswax 3044 PH | Bees Wax |
| | 3.00 | Lanette O | Cetearyl Alcohol |
| | 10.00 | Miglyol 812 | Caprylic/Capric Triglyceride |
| D | 1.00 | Vitamin E Acetate | Tocopheryl Acetate |
| | 0.1% | Resilin protein | |
| | 0.20 | Bisabolol rac. | Bisabolol |

| | | | |
|---|---|---|---|
| A | 3.00 | Glycerin 87% | Glycerin |
| | 0.20 | Edeta BD | Disodium EDTA |
| | 0.30 | Abiol | Imidazolidinyl Urea |
| | 1.00 | Plantacare 2000 | Decyl Glucoside |
| | 0.30 | Keltrol T | Xanthan Gum |
| | 0.05% | Resilin protein | |
| | 2.00 | Mexoryl SL | Terephthalidene Dicamphor |
| | | | Sulfonic Acid |
| | 58.45 | Water dem. | Aqua dem. |
| B | 6.00 | Z-COTE MAX | Zinc Oxide (and) Diphenyl Capryl |
| | | | Methicone |
| | 6.00 | Uvinul N 539 T | Octocrylene |
| | 2.00 | Parsol 1789 | Butyl Methoxydibenzoylmethane |
| | 2.00 | Dow Corning 345 | Cyclopentasiloxane, |
| | | Fluid | Cyclohexasiloxane |
| | 3.50 | Cremophor A 6 | Ceteareth-6, Stearyl Alcohol |
| | 1.50 | Cremophor A 25 | Ceteareth-25 |
| | 0.50 | Beeswax 3044 PH | Bees Wax |
| | 3.00 | Lanette O | Cetearyl Alcohol |
| | 10.00 | Miglyol 812 | Caprylic/Capric Triglyceride |
| D | 1.00 | Vitamin E Acetate | Tocopheryl Acetate |
| | 0.20 | Bisabolol rac. | Bisabolol |

| | | | |
|---|---|---|---|
| A | 3.00 | Glycerin 87% | Glycerin |
| | 0.20 | Edeta BD | Disodium EDTA |
| | 0.30 | Abiol | Imidazolidinyl Urea |
| | 1.00 | Plantacare 2000 | Decyl Glucoside |
| | 0.30 | Keltrol T | Xanthan Gum |
| | 1.0% | Resilin protein | |
| | 2.00 | Neoheliopan AP | Disodium Phenyl Dibenzimidazole |
| | | | Tetrasulfonate |
| | 57.50 | Water dem. | Aqua dem. |
| B | 6.00 | Z-COTE MAX | Zinc Oxide (and) Diphenyl Capryl |
| | | | Methicone |
| | 6.00 | Uvinul N 539 T | Octocrylene |
| | 2.00 | Parsol 1789 | Butyl Methoxydibenzoylmethane |
| | 2.00 | Dow Corning 345 | Cyclopentasiloxane, |
| | | Fluid | Cyclohexasiloxane |
| | 3.50 | Cremophor A 6 | Ceteareth-6, Stearyl Alcohol |
| | 1.50 | Cremophor A 25 | Ceteareth-25 |
| | 0.50 | Beeswax 3044 PH | Bees Wax |
| | 3.00 | Lanette O | Cetearyl Alcohol |
| | 10.00 | Miglyol 812 | Caprylic/Capric Triglyceride |
| D | 1.00 | Vitamin E Acetate | Tocopheryl Acetate |
| | 0.20 | Bisabolol rac. | Bisabolol |

| | | | |
|---|---|---|---|
| A | 25.00 | Dow Corning 345 | Cyclopentasiloxane, |
| | | Fluid | Cyclohexasiloxane |
| | 20.00 | Dow Corning 245 | Cyclopentasiloxane |
| | | Fluid | |
| | 8.00 | Uvinul MC 80 | Ethylhexyl Methoxycinnamate |
| | 4.00 | Abil EM 90 | Cetyl PEG/PPG-10/1 Dimethicone |
| | 7.00 | T-Lite SF | Titanium Dioxide, Alumina Hydrate, |
| | | | Dimethicone/Methicone Copolymer |
| B | 17.00 | Ethanol 95% | Alcohol |
| | 10.00 | Z-COTE MAX | Zinc Oxide (and) Diphenyl Capryl |
| | | | Methicone |
| | 4.50 | Water dem. | Aqua dem. |
| | 3.00 | Glycerin 87% | Glycerin |
| | 0.5% | Resilin protein | |
| | 1.00 | Talc | Talc |

| | | | |
|---|---|---|---|
| A | 25.00 | Dow Corning 345 | Cyclopentasiloxane, |
| | | Fluid | Cyclohexasiloxane |
| | 20.00 | Dow Corning 245 | Cyclopentasiloxane |
| | | Fluid | |
| | 8.00 | Uvinul MC 80 | Ethylhexyl Methoxycinnamate |
| | 4.00 | Abil EM 90 | Cetyl PEG/PPG-10/1 Dimethicone |
| | 7.00 | T-Lite SF-S | Titanium Dioxide, Silica Hydrate, |
| | | | Alumina Hydrate, |
| | | | Methicone/Dimethicone Copolymer |
| B | 17.00 | Ethanol 95% | Alcohol |
| | 9.99 | Z-COTE MAX | Zinc Oxide (and) Diphenyl Capryl |
| | | | Methicone |
| | 5.00 | Water dem. | Aqua dem. |
| | 3.00 | Glycerin 87% | Glycerin |
| | 0.01% | Resilin protein | |
| | 1.00 | Talc | Talc |

| | | | |
|---|---|---|---|
| A | 20.00 | Dow Corning 345 | Cyclopentasiloxane, |
| | | Fluid | Cyclohexasiloxane |
| | 10.00 | Dow Corning 245 | Cyclopentasiloxane |
| | | Fluid | |
| | 7.50 | Uvinul MC 80 | Ethylhexyl Methoxycinnamate |
| | 2.00 | Uvinul T 150 | Ethylhexyl Triazone |
| | 12.00 | Cosmacol EMI | Di-C12-13 Alkyl Malate |
| | 4.00 | Abil EM 90 | Cetyl PEG/PPG-10/1 Dimethicone |
| B | 7.00 | T-Lite SF | Titanium Dioxide, Alumina Hydrate, |
| | | | Dimethicone/Methicone Copolymer |
| C | 15.00 | Ethanol 95% | Alcohol |
| | 10.00 | Z-COTE MAX | Zinc Oxide (and) Diphenyl Capryl |
| | | | Methicone |
| | 3.00 | Glycerin 87% | Glycerin |
| | 1.5% | Resilin protein | |
| | 8.00 | Water dem. | Aqua dem. |

| | | | |
|---|---|---|---|
| A | 20.00 | Dow Corning 345 | Cyclopentasiloxane, |
| | | Fluid | Cyclohexasiloxane |
| | 10.00 | Dow Corning 245 | Cyclopentasiloxane |
| | | Fluid | |
| | 7.50 | Uvinul MC 80 | Ethylhexyl Methoxycinnamate |
| | 2.00 | Tinosorb S | Bis-Ethylhexyloxyphenol |
| | | | Methoxyphenyl Triazine |
| | 12.00 | Cosmacol EMI | Di-C12-13 Alkyl Malate |
| | 4.00 | Abil EM 90 | Cetyl PEG/PPG-10/1 Dimethicone |
| B | 7.00 | T-Lite SF | Titanium Dioxide, Alumina Hydrate, |
| | | | Dimethicone/Methicone Copolymer |
| C | 15.00 | Ethanol 95% | Alcohol |
| | 10.00 | Z-COTE MAX | Zinc Oxide (and) Diphenyl Capryl |
| | | | Methicone |
| | 3.00 | Glycerin 87% | Glycerin |
| | 0.5% | Resilin protein | |
| | 9.00 | Water dem. | Aqua dem. |

| | | | |
|---|---|---|---|
| A | 2.00 | Cremophor WO 7 | PEG-7 Hydrogenated Castor Oil |
| | 6.00 | Abil B 8839 | Cyclopentasiloxane, |
| | | | Cyclohexasiloxane |
| | 3.00 | T-Lite SF | Titanium Dioxide, Alumina Hydrate, |
| | | | Dimethicone/Methicone Copolymer |
| | 4.00 | Isopropyl palmitate | Isopropyl Palmitate |
| | 1.00 | Abil 350 | Dimethicone |
| B | 7.00 | Uvinul MC 80 | Ethylhexyl Methoxycinnamate |
| | 1.00 | Uvinul T 150 | Ethylhexyl Triazone |
| | 7.00 | Finsolv TN | C12-15 Alkyl Benzoate |
| | 4.00 | Abil WE 09 | Polyglyceryl-4 Isostearate, Cetyl |
| | | | PEG/PPG-10/1 Dimethicone, Hexyl |
| | | | Laurate |
| C | 0.50 | Sodium chloride | Sodium Chloride |
| | 1.0% | Resilin protein | |
| | 0.20 | Edeta BD | Disodium EDTA |
| | 61.30 | Water dem. | Aqua dem. |
| D | 1.00 | Vitamin E | Tocopheryl Acetate |
| | | Acetate | |
| | q.s. | Preservative | |

| | | | |
|---|---|---|---|
| A | 2.00 | Cremophor WO 7 | PEG-7 Hydrogenated Castor Oil |
| | 6.00 | Abil B 8839 | Cyclopentasiloxane, |
| | | | Cyclohexasiloxane |
| | 3.00 | Z-COTE MAX | Zinc Oxide (and) Diphenyl Capryl |
| | | | Methicone |
| | 4.00 | Isopropyl palmitate | Isopropyl Palmitate |
| | 1.00 | Abil 350 | Dimethicone |
| B | 7.00 | Uvinul MC 80 | Ethylhexyl Methoxycinnamate |
| | 1.00 | Uvinul T 150 | Ethylhexyl Triazone |
| | 7.00 | Finsolv TN | C12-15 Alkyl Benzoate |
| | 4.00 | Abil WE 09 | Polyglyceryl-4 Isostearate, Cetyl |
| | | | PEG/PPG-10/1 Dimethicone, Hexyl |
| | | | Laurate |
| C | 0.50 | Sodium chloride | Sodium Chloride |
| | 0.1% | Resilin protein | |
| | 0.20 | Edeta BD | Disodium EDTA |
| | 62.20 | Water dem. | Aqua dem. |
| D | 1.00 | Vitamin E Acetate | Tocopheryl Acetate |
| | q.s. | Preservative | |

| | | | |
|---|---|---|---|
| A | 2.00 | Cremophor WO 7 | PEG-7 Hydrogenated Castor Oil |
| | 6.00 | Abil B 8839 | Cyclopentasiloxane, |
| | | | Cyclohexasiloxane |
| | 3.00 | Z-COTE MAX | Zinc Oxide (and) Diphenyl Capryl |
| | | | Methicone |
| | 4.00 | Isopropyl palmitate | Isopropyl Palmitate |
| | 1.00 | Abil 350 | Dimethicone |
| B | 7.00 | Uvinul MC 80 | Ethylhexyl Methoxycinnamate |
| | 1.00 | Tinosorb S | Bis-Ethylhexyloxyphenol |
| | | | Methoxyphenyl Triazine |
| | 7.00 | Finsolv TN | C12-15 Alkyl Benzoate |
| | 4.00 | Abil WE 09 | Polyglyceryl-4 Isostearate, Cetyl |
| | | | PEG/PPG-10/1 Dimethicone, Hexyl |
| | | | Laurate |
| C | 0.50 | Sodium chloride | Sodium Chloride |
| | 2% | Resilin protein | |
| | 0.20 | Edeta BD | Disodium EDTA |
| | 60.30 | Water dem. | Aqua dem. |
| D | 1.00 | Vitamin E Acetate | Tocopheryl Acetate |
| | q.s. | Preservative | |

| | | | |
|---|---|---|---|
| A | 2.00 | Cremophor WO 7 | PEG-7 Hydrogenated Castor Oil |
| | 6.00 | Abil B 8839 | Cyclopentasiloxane, |
| | | | Cyclohexasiloxane |
| | 6.00 | Z-COTE MAX | Zinc Oxide (and)Diphenyl Capryl |
| | | | Methicone |
| | 4.00 | Isopropyl palmitate | Isopropyl Palmitate |
| | 1.00 | Abil 350 | Dimethicone |
| B | 4.00 | Uvinul N 539 T | Octocrylene |
| | 1.00 | Tinosorb S | Bis-Ethylhexyloxyphenol |
| | | | Methoxyphenyl Triazine |
| | 7.00 | Finsolv TN | C12-15 Alkyl Benzoate |
| | 4.00 | Abil WE 09 | Polyglyceryl-4 Isostearate, Cetyl |
| | | | PEG/PPG-10/1 Dimethicone, Hexyl |
| | | | Laurate |
| C | 0.50 | Sodium chloride | Sodium Chloride |
| | 0.2% | Resilin protein | |
| | 0.20 | Edeta BD | Disodium EDTA |
| | 62.10 | Water dem. | Aqua dem. |
| D | 1.00 | Vitamin E Acetate | Tocopheryl Acetate |
| | q.s. | Preservative | |

| | | | |
|---|---|---|---|
| A | 2.00 | Cremophor WO 7 | PEG-7 Hydrogenated Castor Oil |
| | 6.00 | Abil B 8839 | Cyclopentasiloxane, |
| | | | Cyclohexasiloxane |
| | 6.00 | Z-COTE MAX | Zinc Oxide (and) Diphenyl Capryl |
| | | | Methicone |
| | 4.00 | Isopropyl palmitate | Isopropyl Palmitate |
| | 1.00 | Abil 350 | Dimethicone |
| B | 3.00 | Uvinul N 539 T | Octocrylene |
| | 1.00 | Mexoryl XL | Drometrizole Trisiloxane |
| | 1.00 | Tinosorb S | Bis-Ethylhexyloxyphenol |
| | | | Methoxyphenyl Triazine |
| | 7.00 | Finsolv TN | C12-15 Alkyl Benzoate |
| | 4.00 | Abil WE 09 | Polyglyceryl-4 Isostearate, Cetyl |
| | | | PEG/PPG-10/1 Dimethicone, Hexyl |
| | | | Laurate |
| C | 0.50 | Sodium chloride | Sodium Chloride |
| | 0.20 | Edeta BD | Disodium EDTA |
| | 1.0% | Resilin protein | |
| | 61.30 | Water dem. | Aqua dem. |
| D | 1.00 | Vitamin E Acetate | Tocopheryl Acetate |
| | q.s. | Preservative | |

| | | | |
|---|---|---|---|
| A | 2.00 | Cremophor WO 7 | PEG-7 Hydrogenated Castor Oil |
| | 6.00 | Abil B 8839 | Cyclopentasiloxane, |
| | | | Cyclohexasiloxane |
| | 6.00 | T-Lite SF-S | Titanium Dioxide, Silica Hydrate, |
| | | | Alumina Hydrate, |
| | | | Methicone/Dimethicone Copolymer |
| | 4.00 | Isopropyl palmitate | Isopropyl Palmitate |
| | 1.00 | Abil 350 | Dimethicone |
| B | 3.00 | Uvinul N 539 T | Octocrylene |
| | 1.00 | Mexoryl XL | Drometrizole trisiloxane |
| | 1.00 | Tinosorb S | Bis-Ethylhexyloxyphenol |
| | | | Methoxyphenyl Triazine |
| | 7.00 | Finsolv TN | C12-15 Alkyl Benzoate |
| | 4.00 | Abil WE 09 | Polyglyceryl-4 Isostearate, Cetyl |
| | | | PEG/PPG-10/1 Dimethicone, Hexyl |
| | | | Laurate |
| C | 0.50 | Sodium chloride | Sodium Chloride |
| | 0.5% | Resilin protein | |
| | 0.20 | Edeta BD | Disodium EDTA |
| | 61.80 | Water dem. | Aqua dem. |
| D | 1.00 | Vitamin E Acetate | Tocopheryl Acetate |
| | q.s. | Preservative | |

| | | | |
|---|---|---|---|
| A | 4.50 | Uvinul MC 80 | Ethylhexyl Methoxycinnamate |
| | 3.00 | Uvinul N 539 T | Octocrylene |
| | 1.00 | Uvinul T 150 | Ethylhexyl Triazone |
| | 2.50 | Cosmacol EMI | Di-C12-13 Alkyl Malate |
| B | 0.50 | Vitamin E Acetate | Tocopheryl Acetate |
| | 4.00 | Tego Care 450 | Polyglyceryl-3 Methyl Glucose |
| | | | Distearate |
| C | 3.50 | Cetiol SN | Cetearyl Isononanoate |
| | 1.00 | Ganex V-220 | VP/Eicosene Copolymer |
| | 5.00 | Isohexadecane | Isohexadecane |
| | 2.50 | Cosmacol EMI | Di-C12-13 Alkyl Malate |
| | 5.00 | Z-COTE MAX | Zinc Oxide (and) Diphenyl Capryl |
| | | | Methicone |
| D | 44.00 | Water dem. | Aqua dem. |
| | 5.00 | Glycerin 87% | Glycerin |
| | 1.00 | Lanette E | Sodium Cetearyl Sulfate |
| | 15.00 | Luviquat Care | Polyquaternium-44 |
| | 1.0% | Resilin protein | |
| | 0.50 | Keltrol | Xanthan Gum |
| E | 1.00 | Phenonip | |

| | | | |
|---|---|---|---|
| A | 4.50 | Uvinul MC 80 | Ethylhexyl Methoxycinnamate |
| | 3.00 | Uvinul N 539 T | Octocrylene |
| | 1.00 | Tinosorb S | Bis-Ethoxylhexyloxyphenol |
| | | | Methoxyphenyl Triazine |
| | 2.50 | Cosmacol EMI | Di-C12-13 Alkyl Malate |
| B | 0.50 | Vitamin E Acetate | Tocopheryl Acetate |
| | 4.00 | Tego Care 450 | Polyglyceryl-3 Methyl Glucose |
| | | | Distearate |
| C | 3.50 | Cetiol SN | Cetearyl Isononanoate |
| | 1.00 | Ganex V-220 | VP/Eicosene Copolymer |
| | 5.00 | Isohexadecane | Isohexadecane |
| | 2.50 | Cosmacol EMI | Di-C12-13 Alkyl Malate |
| | 5.00 | Z-COTE MAX | Zinc Oxide (and) Diphenyl Capryl |
| | | | Methicone |
| D | 44.90 | Water dem. | Aqua dem. |
| | 5.00 | Glycerin 87% | Glycerin |
| | 1.00 | Lanette E | Sodium Cetearyl Sulfate |
| | 15.00 | Luviquat Care | Polyquaternium-44 |
| | 0.1% | Resilin protein | |
| | 0.50 | Keltrol | Xanthan Gum |
| E | 1.00 | Phenonip | |

| | | | |
|---|---|---|---|
| A | 1.50 | Mexoryl XL | Drometrizole Trisiloxane |
| | 5.00 | Uvinul N 539 T | Octocrylene |
| | 1.00 | Tinosorb S | Bis-Ethylhexyloxyphenol |
| | | | Methoxyphenyl Triazine |
| | 2.50 | Cosmacol EMI | Di-C12-13 Alkyl Malate |
| B | 0.50 | Vitamin E Acetate | Tocopheryl Acetate |
| | 4.00 | Tego Care 450 | Polyglyceryl-3 Methyl Glucose |
| | | | Distearate |
| C | 3.50 | Cetiol SN | Cetearyl Isononanoate |
| | 1.00 | Ganex V-220 | VP/Eicosene Copolymer |
| | 5.00 | Isohexadecane | Isohexadecane |
| | 2.50 | Cosmacol EMI | Di-C12-13 Alkyl Malate |
| | 5.00 | Z-COTE MAX | Zinc Oxide (and) Diphenyl Capryl |
| | | | Methicone |
| D | 44.50 | Water dem. | Aqua dem. |
| | 5.00 | Glycerin 87% | Glycerin |
| | 1.00 | Lanette E | Sodium Cetearyl Sulfate |
| | 15.00 | Luviquat Care | Polyquaternium-44 |
| | 1.5% | Resilin protein | |
| | 0.50 | Keltrol | Xanthan Gum |
| E | 1.00 | Phenonip | |

| | | | |
|---|---|---|---|
| A | 1.50 | Mexoryl XL | Drometrizole Trisiloxane |
| | 5.00 | Uvinul N 539 T | Octocrylene |
| | 1.00 | Tinosorb S | Bis-Ethylhexyloxyphenol |
| | | | Methoxyphenyl Triazine |
| | 2.50 | Cosmacol EMI | Di-C12-13 Alkyl Malate |
| B | 0.50 | Vitamin E Acetate | Tocopheryl Acetate |
| | 4.00 | Tego Care 450 | Polyglyceryl-3 Methyl Glucose |
| | | | Distearate |
| C | 3.50 | Cetiol SN | Cetearyl Isononanoate |
| | 1.00 | Ganex V-220 | VP/Eicosene Copolymer |
| | 5.00 | Isohexadecane | Isohexadecane |
| | 2.50 | Cosmacol EMI | Di-C12-13 Alkyl Malate |
| | 5.00 | T-Lite SF | Titanium Dioxide, Alumina Hydrate, |
| | | | Dimethicone/Methicone Copolymer |
| D | 45.00 | Water dem. | Aqua dem. |
| | 5.00 | Glycerin 87% | Glycerin |
| | 1.00 | Lanette E | Sodium Cetearyl Sulfate |
| | 15.00 | Luviquat Care | Polyquaternium-44 |
| | 1.0% | Resilin protein | |
| | 0.50 | Keltrol | Xanthan Gum |
| E | 1.00 | Phenonip | |

| | | | |
|---|---|---|---|
| A | 1.00 | Abil Care 85 | Bis-PEG/PPG-16/16 PEG/PPG-16/16 |
| | | | Dimethicone, Caprylic/Capric |
| | | | Triglyceride |
| | 3.00 | Cremophor CO 40 | PEG-40 Hydrogenated Castor Oil |
| | 0.30 | Cremophor WO 7 | PEG-7 Hydrogenated Castor Oil |
| | 5.00 | Uvinul MC 80 | Ethylhexyl Methoxycinnamate |
| | 10.00 | Witconol APM | PPG-3 Myristyl Ether |
| | 2.00 | Uvinul T 150 | Ethylhexyl Triazone |
| | 1.00 | Dow Corning 345 | Cyclopentasiloxane, |
| | | Fluid | Cyclohexasiloxane |
| | 5.00 | Uvinul N 539 T | Octocrylene |
| B | 5.00 | Z-COTE MAX | Zinc Oxide (and) Diphenyl Capryl |
| | | | Methicone |
| C | 5.00 | 1,2-Propylene | Propylene Glycol |
| | | glycol | |
| | 0.20 | Keltrol | Xanthan Gum |
| | 0.10 | Edeta BD | Disodium EDTA |
| | 2.0% | Resilin protein | |
| | 1.50 | Simulgel 600 | Acrylamide/Sodium |
| | | | Acryloyldimethyltaurate |
| | | | Copolymer, Isohexadecane, |
| | | | Polysorbate 80 |
| | 58.40 | Water dem. | Aqua dem. |
| D | q.s. | Perfume Oil | |
| | 0.50 | Glidant | DMDM Hydantoin |

| | | | |
|---|---|---|---|
| A | 2.00 | Abil Care 85 | Bis-PEG/PPG-16/16 PEG/PPG-16/16 |
| | | | Dimethicone, Caprylic/Capric |
| | | | Triglyceride |
| | 4.00 | Finsolv TN | C12-15 Alkyl Benzoate |
| | 1.50 | Miglyol 812 | Caprylic/Capric Triglyceride |
| | 0.50 | Vitamin E Acetate | Tocopheryl Acetate |
| | 7.50 | Uvinul MC 80 | Ethylhexyl Methoxycinnamate |
| | 4.00 | Cetiol B | Dibutyl Adipate |
| | 3.00 | Luvitol EHO | Cetearyl Ethylhexanoate |
| | 1.00 | Cremophor CO 40 | PEG-40 Hydrogenated Castor Oil |
| | 1.00 | Paraffin Oil, low | Mineral Oil |
| | | viscosity | |
| | 3.00 | Plantacare 2000 | Decyl Glucoside |
| | 0.50 | Phenonip | |
| | q.s. | Perfume Oil | |
| B | 4.00 | Z-COTE MAX | Zinc Oxide (and) Diphenyl Capryl |
| | | | Methicone |
| C | 2.00 | Simulgel NS | Hydroxyethyl Acrylate/Sodium |
| | | | Acryloyldimethyl Taurate |
| | | | Copolymer, Squalane, |
| | | | Polysorbate 60 |
| | 0.20 | Keltrol | Xanthan Gum |
| | 0.10 | Edeta BD | Disodium EDTA |
| | 1.0% | Resilin protein | |
| | 64.20 | Water dem. | Aqua dem. |

| | | | |
|---|---|---|---|
| A | 6.00 | Gilugel SIL 5 | Cyclomethicone (and) |
| | | | Aluminum/Magnesium |
| | | | Hydroxide Stearate |
| | 5.00 | Uvinul MC 80 | Ethylhexyl Methoxycinnamate |
| | 2.00 | Uvinul A Plus | Diethylamino Hydroxybenzoyl Hexyl |
| | | | Benzoate |
| | 1.00 | Uvinul T 150 | Ethylhexyl Triazone |
| | 7.00 | Finsolv TN | C12-15 Alkyl Benzoate |
| | 4.00 | Abil WE 09 | Polyglyceryl-4 Isostearate, Cetyl |
| | | | PEG/PPG-10/1 Dimethicone, Hexyl |
| | | | Laurate |
| | 2.00 | Cosmacol EMI | Di-C12-13 Alkyl Malate |
| | 3.00 | Isopropyl palmitate | Isopropyl Palmitate |
| | 5.00 | Abil B 8839 | Cyclopentasiloxane, Cyclohexasiloxane |
| | 0.50 | Abil 350 | Dimethicone |
| B | 0.50 | Sodium chloride | Sodium Chloride |
| | 0.20 | Edeta BD | Disodium EDTA |
| | 0.2% | Resilin protein | |
| | 62.10 | Water dem. | Aqua dem. |
| C | 1.00 | Vitamin E Acetate | Tocopheryl Acetate |
| | 0.50 | Phenonip | |

| | | | |
|---|---|---|---|
| A | 4.50 | Uvinul MC 80 | Ethylhexyl Methoxycinnamate |
| | 2.00 | Tinosorb S | Bis-Ethylhexyloxyphenol |
| | | | Methoxyphenyl Triazine |
| | 3.00 | Uvinul N 539 T | Octocrylene |
| | 2.50 | Cosmacol EMI | Di-C12-13 Alkyl Malate |
| | 0.50 | Vitamin E Acetate | Tocopheryl Acetate |
| | 4.00 | Tego Care 450 | Polyglyceryl-3 Methyl Glucose |
| | | | Distearate |
| B | 3.50 | Cetiol SN | Cetearyl Isononanoate |
| | 1.00 | Ganex V-220 | VP/Eicosene Copolymer |
| | 5.00 | Isohexadecane | Isohexadecane |
| | 2.50 | Cosmacol EMI | Di-C12-13 Alkyl Malate |
| | 3.00 | T-Lite SF | Titanium Dioxide, Alumina Hydrate, |
| | | | Dimethicone/Methicone Copolymer |
| C | 5.00 | Glycerin 87% | Glycerin |
| | 1.00 | Lanette E | Sodium Cetearyl Sulfate |
| | 0.50 | Keltrol | Xanthan Gum |
| | 1.0% | Resilin protein | |
| | 59.70 | Water dem. | Aqua dem. |
| D | 1.00 | Phenonip | |
| | 0.30 | Bisabolol rac. | Bisabolol |

| | | | |
|---|---|---|---|
| A | 2.00 | Abil Care 85 | Bis-PEG/PPG-16/16 PEG/PPG-16/16 |
| | | | Dimethicone, Caprylic/Capric |
| | | | Triglyceride |
| | 4.00 | Finsolv TN | C12-15 Alkyl Benzoate |
| | 1.50 | Miglyol 812 | Caprylic/Capric Triglyceride |
| | 0.50 | Vitamin E Acetate | Tocopheryl Acetate |
| | 7.50 | Uvinul MC 80 | Ethylhexyl Methoxycinnamate |
| | 4.00 | Cetiol B | Dibutyl Adipate |
| | 1.00 | Luvitol EHO | Cetearyl Ethylhexanoate |
| | 1.00 | Cremophor CO 40 | PEG-40 Hydrogenated Castor Oil |
| | 1.00 | Paraffin Oil, low | Mineral Oil |
| | | viscosity | |
| | 3.00 | Plantacare 2000 | Decyl Glucoside |
| | 1.00 | Uvinul A Plus | Diethylamino Hydroxybenzoyl Hexyl |
| | | | Benzoate |
| | 0.50 | Phenonip | |
| | 2.50 | Uvinul T 150 | Ethylhexyl Triazone |
| | q.s. | Perfume Oil | |
| B | 4.00 | Z-COTE MAX | Zinc Oxide (and) Diphenyl Capryl |
| | | | Methicone |
| C | 2.00 | Simulgel NS | Hydroxyethyl Acrylate/Sodium |
| | | | Acryloyldimethyl Taurate Copolymer, |
| | | | Squalane, Polysorbate 60 |
| | 0.10 | Keltrol | Xanthan Gum |
| | 0.10 | Edeta BD | Disodium EDTA |
| | 0.5% | Resilin protein | |
| | 59.90 | Water dem. | Aqua dem. |

| | | | |
|---|---|---|---|
| A | 4.00 | Eumulgin VL 75 | Lauryl Glucoside, Polyglyceryl-2 |
| | | | Dipolyhydroxystearate, Glycerin |
| | 2.00 | Lanette O | Cetearyl Alcohol |
| | 12.00 | Myritol 331 | Cocoglycerides |
| | 8.00 | Finsolv TN | C12-15 Alkyl Benzoate |
| | 8.00 | Cetiol B | Dibutyl Adipate |
| B | 6.00 | T-Lite SF | Titanium Dioxide, Alumina Hydrate, |
| | | | Dimethicone/Methicone Copolymer |
| | 5.00 | Z-COTE MAX | Zinc Oxide (and) Diphenyl Capryl |
| | | | Methicone |
| C | 3.00 | Glycerin 87% | Glycerin |
| | 0.10 | Edeta BD | Disodium EDTA |
| | 1.50 | Veegum Ultra | Magnesium Aluminum Silicate |
| | 1.50 | Lanette E | Sodium Cetearyl Sulfate |
| | 2% | Resilin protein | |
| | 0.30 | Keltrol | Xanthan Gum |
| | 45.10 | Water dem. | Water |
| D | 1.00 | Phenonip | Phenoxyethanol, Methylparaben, |
| | | | Ethylparaben, Butylparaben, |
| | | | Propylparaben, Isobutylparaben |

| | | | |
|---|---|---|---|
| A | 4.00 | Eumulgin VL 75 | Lauryl Glucoside, Polyglyceryl-2 |
| | | | Dipolyhydroxystearate, Glycerin |
| | 2.00 | Lanette O | Cetearyl Alcohol |
| | 12.00 | Myritol 331 | Cocoglycerides |
| | 8.00 | Finsolv TN | C12-15 Alkyl Benzoate |
| | 8.00 | Cetiol B | Dibutyl Adipate |
| B | 6.00 | Uvinul N 539 T | Octocrylene |
| | 5.00 | Z-COTE MAX | Zinc Oxide (and) Diphenyl Capryl |
| | | | Methicone |
| C | 3.00 | Glycerin 87% | Glycerin |
| | 0.10 | Edeta BD | Disodium EDTA |
| | 1.50 | Veegum Ultra | Magnesium Aluminum Silicate |
| | 1.50 | Lanette E | Sodium Cetearyl Sulfate |
| | 0.30 | Carbopol Ultrez 10 P | Carbomer |
| | 1.0% | Resilin protein | |
| | 46.10 | Water dem. | Aqua dem. |
| D | 1.00 | Phenonip | Phenoxyethanol, Methylparaben, |
| | | | Ethylparaben, Butylparaben, |
| | | | Propylparaben, Isobutylparaben |

| | | |
|---|---|---|
| A | 4.00 Eumulgin VL 75 | Lauryl Glucoside, Polyglyceryl-2 |
| | | Dipolyhydroxystearate, Glycerin |
| | 2.00 Lanetta 0 | Cetearyl Alcohol |
| | 10.00 Myritol 331 | Cocoglycerides |
| | 8.00 Finsolv TN | C12-15 Alkyl Benzoate |
| | 8.00 Cetiol B | Dibutyl Adipate |
| B | 2.00 Uvinul A Plus | Diethylamino Hydroxybenzoyl Hexyl |
| | | Benzoate |
| | 5.00 Z-COTE MAX | Zinc Oxide (and) Diphenyl Capryl |
| | | Methicone |
| C | 3.00 Glycerin 87% | Glycerin |
| | 0.10 Edeta BD | Disodium EDTA |
| | 1.50 Veegum Ultra | Magnesium Aluminum Silicate |
| | 1.50 Lanette E | Sodium Cetearyl Sulfate |
| | 5.0% Resilin protein | |
| | 0.30 Carbopol Ultrez 10 P | Carbomer |
| | ad Water dem. | Water |
| D | 1.00 Phenonip | Phenoxyethanol, Methylparaben, |
| | | Ethylparaben, Butylparaben, |
| | | Propylparaben, Isobutylparaben |

| | | |
|---|---|---|
| A | 3.50 Cremophor A 6 | Ceteareth-6, Stearyl Alcohol |
| | 1.50 Cremophor A 25 | Ceteareth-25 |
| | 7.50 Uvinul MC 80 | Ethylhexyl Methoxycinnamate |
| | 2.00 Uvinul A Plus | Diethylamino Hydroxybenzoyl Hexyl |
| | | Benzoate |
| | 2.00 Dow Corning 345 | Cyclopentasiloxane, Cyclohexasiloxane |
| | | Fluid |
| | 0.50 Beeswax 3044 PH | Bees Wax |
| | 3.00 Lanette O | Cetearyl Alcohol |
| | 10.00 Miglyol 812 | Caprylic/Capric Triglyceride |
| B | 5.00 T-Lite SF-S Titanium | Titanium Dioxide, Silica Hydrate, |
| | | Alumina Hydrate, Methicone/ |
| | | Dimethicone Copolymer |
| C | 3.00 Glycerin 87% | Glycerin |
| | 0.20 Edeta BD | Disodium EDTA |
| | 0.30 Keltrol T | Xanthan Gum |
| | 1.00 Plantacare 2000 | Decyl Glucoside |
| | 2.0% Resilin protein | |
| | 57.30 Water dem. | Aqua dem. |
| D | 1.00 Vitamin E Acetate | Tocopheryl Acetate |
| | 0.20 Bisabolol rac. | Bisabolol |

| | | |
|---|---|---|
| A | 10.00 Uvinul A Plus B | Ethylhexyl Methoxycinnamate, |
| | | Diethylamino Hydroxybenzoyl |
| | | Hexyl Benzoate |
| | 10.00 Uvinul N 539 T | Octocrylene |
| | 4.00 Eumulgin VL 75 | Lauryl Glucoside, Polyglyceryl-2 |
| | | Dipolyhydroxystearate, Glycerin |
| | 8.00 Cetiol B | Dibutyl Adipate |
| | 8.00 Finsolv TN | C12-15 Alkyl Benzoate |
| | 12.00 Myritol 331 | Cocoglycerides |
| | 1.00 Lanette E | Sodium Cetearyl Sulfate |
| | 2.00 Lanette O | Cetearyl Alcohol |
| B | 3.00 Z-COTE MAX | Zinc Oxide (and) Diphenyl Capryl |
| | | Methicone |
| C | 35.08 Water dem. | Aqua dem. |
| | 0.38 Citric acid | Citric Acid |
| | 2.9 Glycerin 87% | Glycerin |
| | 0.05 Edeta BD | Disodium EDTA |
| | 0.20 Atlantoin | Allantoin |
| | 0.30 Keltrol | Xanthan Gum |
| | 0.1% Resilin protein | |
| | 1.50 Veegum Ultra | Magnesium Aluminum Silicate |
| D | 0.50 Phenonip | |
| | 1.00 Vitamin E-Acetate | Tocopheryl Acetate |

| | | |
|---|---|---|
| A | 0.70 Cremophor A 25 | Ceteareth-25 |
| | 1.70 Cremophor A 6 | Ceteareth-6, Stearyl Alcohol |
| | 2.00 Uvinul A Plus | Diethylamino Hydroxybenzoyl Hexyl |
| | | Benzoate |
| | 3.00 Uvinul N 539 T | Octocrylene |
| | 3.00 Z-COTE MAX | Zinc Oxide (and) Diphenyl Capryl |
| | | Methicone |
| | 2.00 Abil B 8843 | PEG-14 Dimethicone |
| | 3.60 Lanette O | Cetearyl Alcohol |
| | 4.00 Uvinul MC 80 | Ethylhexyl Methoxycinnamate |
| | 2.00 Cetiol B | Dibutyl Adipate |
| B | 4.00 Glycerin 87% | Glycerin |
| | 1.0% Resilin protein | |
| | 0.20 Edeta BD | Disodium EDTA |
| | 71.00 Water dem. | Panthenol |
| C | 4.00 Luvigel EM | Caprylic/Capric Triglyceride, |
| | | Sodium Acrylates Copolymer |
| D | 1.00 Vitamin E Acetate | Tocopheryl Acetate |
| | 0.20 Bisabolol rac. | Bisabolol |
| | q.s. Preservative | |

| | | |
|---|---|---|
| A | 7.50 Uvinul MC 80 | Ethylhexyl Methoxycinnamate |
| | 5.00 Uvinul N 539 T | Octocrylene |
| | 3.00 Emulgade PL 68/50 | Cetearyl Glucoside, Cetearyl Alcohol |
| | 2.00 Dracorin 100 SE | Glyceryl Stearate, PEG-100 Stearate |
| | 1.00 Fitoderm | Squalane |
| | 0.50 Cremophor WO 7 | PEG-7 Hydrogenated Castor Oil |
| | 0.50 Cremophor PS 20 | Polysorbate 20 |
| | 2.00 Dry Flo Pure | Aluminum Starch Octenylsuccinate |
| B | 5.00 Z-COTE MAX | Zinc Oxide (and) Diphenyl Capryl |
| | | Methicone |
| | 0.03 Sicomet Blue P 77 007 | C.I. 77 007, Ultramarines |
| C | 4.00 1,2-Propylene Glycol | Propylene Glycol |
| | Care | |
| | 2.00 D-Panthenol 50 P | Panthenol, Propylene Glycol |
| | 0.20 Keltrol | Xanthan Gum |
| | 0.50 Simulgel 600 | Acrylamide/Sodium |
| | | Acryloyldimethyltaurate Copolymer, |
| | | Isohexadecane, Polysorbate 80 |
| | 0.5% Resilin protein | |
| | 65.27 Water dem. | Aqua dem. |
| | q.s. Preservative | |
| | 0.50 Vitamin E-Acetate | Tocopheryl Acetate |

| | | |
|---|---|---|
| A | 6.00 Gilugel SIL 5 | Cyclomethicone (and) Aluminium/ |
| | | Magnesium Hydroxide Stearate |
| | 5.00 Uvinul N 539 T | Octocrylene |
| | 2.00 Mexoryl XL | Drometrizole Trisiloxane |
| | 1.00 Uvinul T 150 | Ethylhexyl Triazone |
| | 3.00 T-Lite SF-S | Titanium Dioxide, Silica Hydrate, |
| | | Alumina Hydrate, Methicone/ |
| | | Dimethicone Copolymer |
| | 5.00 Finsolv TN | C12-15 Alkyl Benzoate |
| | 4.00 Abil WE 09 | Polyglyceryl-4 Isostearate, Cetyl |
| | | PEG/PPG-10/1 Dimethicone, Hexyl |
| | | Laurate |
| | 2.00 Cosmacol EMI | Di-C12-13 Alkyl Malate |
| | 3.00 Isopropyl palmitate | Isopropyl Palmitate |
| | 5.00 Abil B 8839 | Cyclopentasiloxane, Cyclohexasiloxane |
| | 0.50 Abil 350 | Dimethicone |
| B | 0.50 Sodium chloride | Sodium Chloride |
| | 0.20 Edeta BD | Disodium EDTA |
| | 1.0% Resilin protein | |
| | 61.30 Water dem. | Aqua dem. |
| C | 1.00 Vitamin E Acetate | Tocopheryl Acetate |
| | 0.50 Phenonip | |

| | | |
|---|---|---|
| A | 1.00 Abil Care 85 | Bis-PEG/PPG-16/16 PEG/PPG-16/16 |
| | | Dimethicone, Caprylic/Capric |
| | | Triglyceride |
| | 3.00 Cremophor CO 40 | PEG-40 Hydrogenated Castor Oil |
| | 0.30 Cremophor WO 7 | PEG-7 Hydrogenated Castor Oil |
| | 5.00 Uvinul N 539 T | Octocrylene |
| | 10.00 Witconol APM | PPG-3 Myristyl Ether |
| | 2.00 Uvinul T 150 | Ethylhexyl Triazone |
| | 1.00 Dow Corning 345 Fluid | Cyclopentasiloxane, |
| | | Cyclohexasiloxane |
| | 2.00 Uvinul A Plus | Diethylamino Hydroxybenzoyl |
| | | Hexyl Benzoate |
| B | 5.00 Z-COTE MAX | Zinc Oxide (and) Diphenyl |
| | | Capryl Silane |
| C | 5.00 1,2-Propylene glycol | Propylene Glycol |
| | 0.20 Keltrol | Xanthan Gum |
| | 0.10 Edeta BD | Disodium EDTA |
| | 1.0% Resilin protein | |
| | 1.50 Simulgel 600 | Acrylamide/Sodium Acryloyldi- |
| | | methyltaurate Copolymer, |
| | | Isohexadecane, Polysorbate 80 |
| | 59.40 Water dem. | Aqua dem. |
| D | q.s. Perfume Oil | |
| | 0.50 Glidant | DMDM Hydantoin |

| | | |
|---|---|---|
| A | 7.00 Uvinul A Plus B | Ethylhexyl Methoxycinnamate, |
| | | Diethylamino Hydroxybenzoyl |
| | | Hexyl Benzoate |
| | 1.00 Tinosorb S | Bis-Ethylhexyloxyphenol |
| | | Methoxyphenyl Triazine |
| | 1.00 Uvinul T 150 | Ethylhexyl Triazone |
| | 7.00 Uvinul N 539 T | Octocrylene |
| | 4.00 Eumulgin VL 75 | Lauryl Glucoside, Polyglyceryl-2 |
| | | Dipolyhydroxystearate, Glycerin |
| | 8.00 Cetiol B | Dibutyl Adipate |
| | 8.00 Finsolv TN | C12-15 Alkyl Benzoate |
| | 12.00 Myritol 331 | Cocoglycerides |
| | 1.00 Lanette E | Sodium Cetearyl Sulfate |
| | 2.00 Lanette O | Cetearyl Alcohol |
| B | 5.00 Z-COTE MAX | Zinc Oxide (and) Diphenyl Capryl |
| | | Methicone |
| C | 35.35 Water dem. | Aqua dem. |
| | 3.00 Glycerin 87% | Glycerin |
| | 0.05 Edeta BD | Disodium EDTA |
| | 0.20 Allantoin | Allantoin |
| | 0.1% Resilin protein | |
| | 0.30 Keitrol | Xanthan Gum |
| | 1.50 Veegum Ultra | Magnesium Aluminum Silicate |
| D | 0.50 Phenonip | |
| | 1.00 Vitamin E Acetate | Tocopheryl Acetate |

| | | |
|---|---|---|
| A | 7.00 Uvinul A Plus B | Ethylhexyl Methoxycinnamate, |
| | | Diethylamino Hydroxybenzoyl |
| | | Hexyl Benzoate |
| | 1.00 Tinosorb S | Bis-Ethylhexyloxyphenol |
| | | Methoxyphenyl Triazine |
| | 1.00 Uvinul T 150 | Ethylhexyl Triazone |
| | 7.00 Uvinul N 539 T | Octocrylene |
| | 4.00 Eumulgin VL 75 | Lauryl Glucoside, Polyglyceryl-2 |
| | | Dipolyhydroxystearate, Glycerin |
| | 8.00 Cetiol B | Dibutyl Adipate |
| | 8.00 Finsolv TN | C12-15 Alkyl Benzoate |
| | 12.00 Myritol 331 | Cocoglycerides |
| | 1.00 Lanette E | Sodium Cetearyl Sulfate |
| | 2.00 Lanette O | Cetearyl Alcohol |
| B | 5.00 T-Lite SF | Titanium Dioxide, Alumina Hydrate, |
| | | Dimethicone/Methicone Copolymer |
| C | 30.45 Water dem. | Aqua dem. |
| | 3.00 Glycerin 87% | Glycerin |
| | 0.05 Edeta BD | Disodium EDTA |
| | 0.20 Allantoin | Allantoin |
| | 0.30 Keltrol | Xanthan Gum |
| | 5.0% Resilin protein | |
| | 1.50 Veegum Ultra | Magnesium Aluminum Silicate |
| D | 0.50 Phenonip | |
| | 1.00 Vitamin E Acetate | Tocopheryl Acetate |

| | |
|---|---|
| 5.00 Z-COTE MAX | Zinc Oxide (and) Diphenyl Capryl |
| | Methicone |
| 2.00 Uvinul A Plus | Diethylamino Hydroxybenzoyl Hexyl |
| | Benzoate |
| 2.00 Mexoryl XL | Drometrizole Trisiloxane |
| 3.00 Uvinul MC 80 | Ethylhexyl Methoxycinnamate |
| 0.50 Abil 350 | Dimethicone |
| 2.75 Carnico wax LT 20 | Carnauba (Copernica Cerifera) Wax, |
| | Paraffin |
| 3.70 Candelilla wax LT 281 LJ | Candelilla (Euphorbia Cerifera) Wax |
| 1.80 Beeswax 3050 PH | Bees Wax |
| 3.20 TeCero wax 30445 | Microcrystalline Wax |
| 3.20 TeCero wax 1030 K | Microcrystalline Wax |
| 1.34 Cutina CP | Cetyl Palmitate |
| 6.40 Vaseline | Petrolatum |
| 7.30 Softisan 100 | Hydrogenated Coco-Glycerides |
| 10.00 Luvitol EHO | Cetearyl Ethylhexanoate |
| 0.17 Bisabolol nat. | Bisabolol |
| 1.84 Vitamin E Acetate | Tocopheryl Acetate |
| 1.0% Resilin protein | |
| 0.42 D,L-Alpha-Tocopherol | Tocopherol |
| 40.38 Castor oil | Castor (Ricinus Communis) Oil |

| | | |
|---|---|---|
| A | 1.00 Abil Care 85 | Bis-PEG/PPG-16/16 PEG/PPG-16/16 |
| | | Dimethicone, Caprylic/Capric |
| | | Triglyceride |
| | 3.00 Cremophor CO 40 | PEG-40 Hydrogenated Castor Oil |
| | 0.30 Cremophor WO 7 | PEG-7 Hydrogenated Castor Oil |
| | 2.00 Mexoryl XL | Drometrizole Trisiloxane |
| | 10.00 Witconol APM | PPG-3 Myristyl Ether |
| | 1.00 Uvinul T 150 | Ethylhexyl Triazone |
| | 1.00 Dow Corning 345 | Cyclopentasiloxane, |
| | Fluid | Cyclohexasiloxane |
| | 5.00 Uvinul N 539 T | Octocrylene |
| B | 3.00 T-Lite SF-S | Titanium Dioxide, Silica Hydrate, |
| | | Alumina Hydrate, Methicone/ |
| | | Dimethicone Copolymer |
| C | 5.00 1,2-Propylene | Propylene Glycol |
| | glycol | |
| | 1.00 Mexoryl SX | Terephthalidene Dicamphor |
| | | Sulfonic Acid |
| | 0.20 Keltrol | Xanthan Gum |
| | 0.10 Edeta BD | Disodium EDTA |
| | 1.0% Resilin protein | |
| | 1.50 Simulgel 600 | Acrylamide/Sodium Acryloyl- |
| | | dimethyltaurate Copolymer, |
| | | Isohexadecane, Polysorbate 80 |
| | 69.50 Water dem. | Aqua dem. |
| D | q.s. Perfume Oil | |
| | 0.50 Glidant | DMDM Hydantoin |

| | | |
|---|---|---|
| A | 1.00 Abil Care 85 | Bis-PEG/PPG-16/16 PEG/PPG-16/16 |
| | | Dimethicone, Caprylic/Capric |
| | | Triglyceride |
| | 3.00 Cremophor CO 40 | PEG-40 Hydrogenated Castor Oil |
| | 0.30 Cremophor WO 7 | PEG-7 Hydrogenated Castor Oil |
| | 2.00 Parsol 1789 | Butyl Methoxydibenzoylmethane |
| | 2.00 Mexoryl XL | Drometrizole Trisiloxane |
| | 10.00 Witconol APM | PPG-3 Myristyl Ether |
| | 1.00 Uvinul T 150 | Ethylhexyl Triazone |
| | 1.00 Dow Corning 345 | Cyclopentasiloxane, |
| | Fluid | Cyclohexasiloxane |
| | 5.00 Uvinul N 539 T | Octocrylene |
| B | 3.00 T-Lite SF-S | Titanium Dioxide, Silica Hydrate, |
| | | Alumina Hydrate, Methicone/ |
| | | Dimethicone Copolymer |
| C | 5.00 1,2-Propylene | Propylene Glycol |
| | glycol | |
| | 1.00 Mexoryl SX | Terephthalidene Dicamphor |
| | | Sulfonic Acid |
| | 0.20 Keltrol | Xanthan Gum |
| | 0.10 Edeta BD | Disodium EDTA |
| | 0.2% Resilin protein | |
| | 1.50 Simulgel 600 | Acrylamide/Sodium Acryloyl- |
| | | dimethyltaurate Copolymer, |
| | | Isohexadecane, Polysorbate 80 |
| | 68.30 Water dem. | Aqua dem. |
| D | q.s. Perfume Oil | |
| | 0.50 Glidant | DMDM Hydantoin |

| | | |
|---|---|---|
| A | 0.70 Cremophor A 25 | Ceteareth-25 |
| | 1.70 Cremophor A 6 | Ceteareth-6, Stearyl Alcohol |
| | 2.00 Parsol 1789 | Butyl Methoxydibenzoylmethane |
| | 5.00 Uvinul N 539 T | Octocryiene |
| | 4.00 Z-COTE MAX | Zinc Oxide (and) Diphenyl Capryl |
| | | Methicone |
| | 2.00 Abil B 8843 | PEG-14 Dimethicone |
| | 3.60 Lanette O | Cetearyl Alcohol |
| | 1.00 Uvinul T150 | Ethylhexyl Triazone |
| | 2.00 Cetiol B | Dibutyl Adipate |
| B | 4.00 Glycerin 87% | Glycerin |
| | 0.20 Edeta BD | Disodium EDTA |
| | 71.00 Water dem. | Panthenol |
| C | 4.00 Luvigel EM | Caprylic/Capric Triglyceride, |
| | | Sodium Acrylates Copolymer |
| | 1.0% Resilin protein | |
| D | 1.00 Vitamin E Acetate | Tocopheryl Acetate |
| | 0.20 Bisabolol rac. | Bisabolol |
| | q.s. Preservative | |

| | | |
|---|---|---|
| A | 1.00 Abil Care 85 | Bis-PEG/PPG-16/16 PEG/PPG-16/16 |
| | | Dimethicone, Caprylic/Capric |
| | | Triglyceride |
| | 3.00 Cremophor CO 40 | PEG-40 Hydrogenated Castor Oil |
| | 0.30 Cremophor WO 7 | PEG-7 Hydrogenated Castor Oil |
| | 2.00 Parsol 1789 | Butyl Methoxydibenzoylmethane |
| | 2.00 Mexoryl XL | Drometrizole Trisiloxane |
| | 10.00 Witconol APM | PPG-3 Myristyl Ether |
| | 1.00 Uvinul T 150 | Ethylhexyl Triazone |
| | 1.00 Dow Corning 345 Fluid | Cyclopentasiloxane, |
| | | Cyclohexasiloxane |
| | 5.00 Uvinul N 539 T | Octocrylene |
| B | 3.00 Z-COTE MAX | Zinc Oxide (and) Diphenyl Capryl |
| | | Methicone |
| C | 5.00 1,2-Propylene glycol | Propylene Glycol |
| | 1.00 Mexoryl SX | Terephthalidene Dicamphor |
| | | Sulfonic Acid |
| | 0.20 Keltrol | Xanthan Gum |
| | 0.10 Edeta BD | Disodium EDTA |
| | 1.50 Simulgel 600 | Acrylamide/Sodium Acryloyl- |
| | | dimethyltaurate Copolymer, |
| | | Isohexadecane, Polysorbate 80 |
| | 0.5% Resilin protein | |
| | 68.00 Water dem. | Aqua dem. |
| D | q.s. Perfume Oil | |
| | 0.50 Glidant | DMDM Hydantoin |

| | | |
|---|---|---|
| A | 2.00 Uvinul A Plus | Diethylamino Hydroxybenzoyl Hexyl |
| | | Benzoate |
| | 3.00 Uvinul N 539 T | Octocrylene |
| | 3.00 Emulgade PL 68/50 | Cetearyl Glucoside, Cetearyl Alcohol |
| | 2.00 Dracorin 100 SE | Glyceryl Stearate, PEG-100 Stearate |
| | 1.00 Fitoderm | Squalane |
| | 0.5 Cremophor WO 7 | PEG-7 Hydrogenated Castor Oil |
| | 0.5 Cremophor PS 20 | Polysorbate 20 |
| | 2.00 Dry Flo Pure | Aluminium Starch Octenylsuccinate |
| B | 5.00 Z-COTE MAX | Zinc Oxide (and) Diphenyl Capryl |
| | | Methicone |
| C | 4.00 1,2-Propylene | Propylene Glycol |
| | Glycol Care | |
| | 0.20 Keltrol | Xanthan Gum |
| | 1.0% Resilin protein | |
| | 0.50 Simulgel 600 | Acrylamide/Sodium Acryloyl- |
| | | dimethyltaurate Copolymer, |
| | | isohexadecane, Polysorbate 80 |
| | 65.80 Water dem. | Aqua dem. |
| D | 0.50 Euxyl K 300 | |
| | 0.50 Vitamin E- Acetate | Tocopheryl Acetate |
| | 1.00 RetiSTAR | Caprylic/Capric Triglyceride, Sodium |
| | | Ascorbate, Tocopherol, Retinol |

### DESCRIPTION OF THE FIGURES

FIG. 1 Analysis of the purified U₁₆ protein by means of SDS-PAGE: (A) Coomassie staining (B) detection with anti-T7 antibodies after Western Blot. The antibody binds the T7 peptide tag localized on the N terminus of the repetitive sequence of the U₁₆. Trace 1: purified U₁₆ protein; trace 2: protein standard marker; MW: molecular weights of the corresponding marker bands.

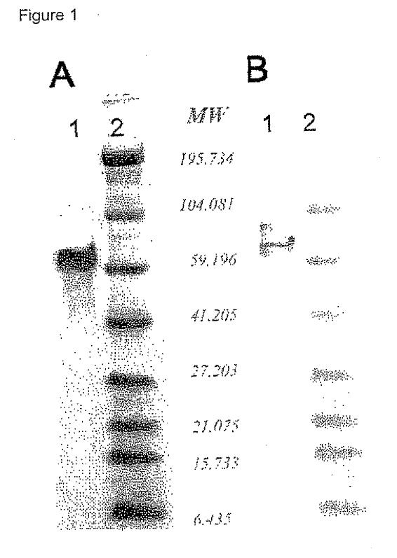

FIG. 2 Analysis of the purity of the purified U₁₆ by means of fluorescence spectroscopy. U₁₆ does not comprise the amino acid tryptophan, whereas tyrosines are present. By contrast, tryptophan is present in proteins of E. coli with an average frequency of 1.5%. Through fluorescence spectroscopy it is possible to selectively detect the presence of tryptophans in a protein sample and thus of impurities in U₁₆ preparations. Whereas both tyrosines and tryptophans are excited by light of wavelength 280 nm, light with 295 nm leads exclusively to the excitation of tryptophan. The U₁₆ sample shows a fluorescence typical of tyrosine, whereas no sign of the presence of tryptophan can be detected, which is an indication of the high purity of the protein sample.

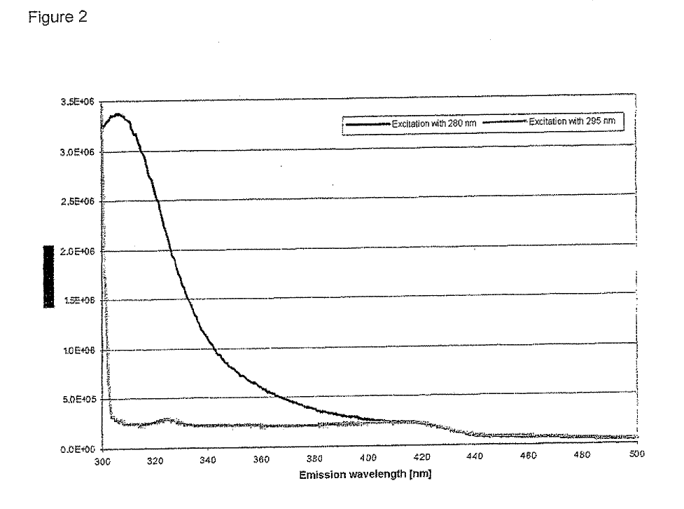

1-10. (canceled)

11. A method for producing a cosmetic composition comprising utilizing a resilin protein in the production of a cosmetic composition, wherein the resilin protein comprises between one and 100 repeat units with the consensus sequence SXXYGXP (SEQ ID NO: 4), where S is a serine, X is any amino acid, Y is a tyrosine, G is a glycine, and P is a proline.

12. The method of claim 11, wherein the repeat units comprise the consensus sequence:

| | |
|---|---|
| X₄SX₅X₆YGX₇PX₈X₉X₁₀ | (SEQ ID NO: 9) |

where
X₄ is a proline alanine, threonine or serine
S is a serine
X₅ is a charged or polar amino acid
X₆ is a charged or polar amino acid
Y is a tyrosine
G is a glycine
X₇ is a proline, alanine, threonine or serine
P is a proline
X₈ is a glycine, alanine, threonine or scrine
X₉ is a glycine, a polar amino acid or not an amino acid
X₁₀ is a glycine, a polar amino acid or not an amino acid.

13. The method of claim 11, wherein the composition comprises a hybrid protein comprising resilin sequences and sequences of non-resilin-structural proteins.

14. The method of claim 11, wherein the resilin protein is joined to an effector molecule.

15. The method of claim 14, wherein the effector molecule is a UV absorber.

16. The method of claim 11, wherein the resilin protein is used in skin cosmetics.

17. A cosmetic composition comprising at least one resilin protein and at least one active ingredient from the group of UV absorbers.

18. A basic resilin protein, wherein the ratio of basic amino acid radicals (K, R) present in the repeat units which form the resilin protein to acidic amino acid radicals (D, E) is at least 1.5.

19. The basic resilin protein of claim 18, wherein the resilin protein comprises the sequence as set forth in SEQ ID NO: 1.

20. A method of producing the basic resilin protein of claim 18, comprising producing a gene sequence which codes for a repeat unit of a basic resilin protein, combining one or more repeat units to give an overall gene with one or more repeat units, expressing the overall gene in a host organism, and isolating the expressed basic resilin protein from the host organism and/or its culture medium.


SEQUENCE LISTING (9 sequences)

SEQ ID NO: 1 - protein, 496 aa, Artificial Sequence
  synthetic sequence
  MISC_FEATURE (1)..(15): T7 TAG
MASMTGGQQMGRGSMGAPGGGNGGRPSDTYGAPGGGNGGRPSSSYGAPGGGNGGRPSDTYGAPGGGNGGRPSSSYGAPGGGNGGRPSDTYGAPGGGNGGRPSSSYGAPGGGNGGRPSDTYGAPGGGNGGRPSSSYGAPGGGNGGRPSDTYGAPGGGNGGRPSSSYGAPGGGNGGRPSDTYGAPGGGNGGRPSSSYGAPGGGNGGRPSDTYGAPGGGNGGRPSSSYGAPGGGNGGRPSDTYGAPGGGNGGRPSSSYGAPGGGNGGRPSDTYGAPGGGNGGRPSSSYGAPGGGNGGRPSDTYGAPGGGNGGRPSSSYGAPGGGNGGRPSDTYGAPGGGNGGRPSSSYGAPGGGNGGRPSDTYGAPGGGNGGRPSSSYGAPGGGNGGRPSDTYGAPGGGNGGRPSSSYGAPGGGNGGRPSDTYGAPGGGNGGRPSSSYGAPGGGNGGRPSDTYGAPGGGNGGRPSSSYGAPGGGNGGRPSDTYGAPGGGNGGRPSSSYG

SEQ ID NO: 2 - DNA, 1440 bp, Artificial Sequence
  Synthetic sequence:  genetically recombined sequence
ggtgcgccgggcggtggcaacggtggccgtccgtctgacacctacggtgcgccgggtggcggtaacggtggccgtccttcttcctcttacggtgcgccgggcggtggcaacggtggccgtccgtctgacacctacggtgcgccgggtggcggtaacggtggccgtccttcttcctcttacggtgcgccgggcggtggcaacggtggccgtccgtctgacacctacggtgcgccgggtggcggtaacggtggccgtccttcttcctcttacggtgcgccgggcggtggcaacggtggccgtccgtctgacacctacggtgcgccgggtggcggtaacggtggccgtccttcttcctcttacggtgcgccgggcggtggcaacggtggccgtccgtctgacacctacggtgcgccgggtggcggtaacggtggccgtccttcttcctcttacggtgcgccgggcggtggcaacggtggccgtccgtctgacacctacggtgcgccgggtggcggtaacggtggccgtccttcttcctcttacggtgcgccgggcggtggcaacggtggccgtccgtctgacacctacggtgcgccgggtggcggtaacggtggccgtccttcttcctcttacggtgcgccgggcggtggcaacggtggccgtccgtctgacacctacggtgcgccgggtggcggtaacggtggccgtccttcttcctcttacggtgcgccgggcggtggcaacggtggccgtccgtctgacacctacggtgcgccgggtggcggtaacggtggccgtccttcttcctcttacggtgcgccgggcggtggcaacggtggccgtccgtctgacacctacggtgcgccgggtggcggtaacggtggccgtccttcttcctcttacggtgcgccgggcggtggcaacggtggccgtccgtctgacacctacggtgcgccgggtggcggtaacggtggccgtccttcttcctcttacggtgcgccgggcggtggcaacggtggccgtccgtctgacacctacggtgcgccgggtggcggtaacggtggccgtccttcttcctcttacggtgcgccgggcggtggcaacggtggccgtccgtctgacacctacggtgcgccgggtggcggtaacggtggccgtccttcttcctcttacggtgcgccgggcggtggcaacggtggccgtccgtctgacacctacggtgcgccgggtggcggtaacggtggccgtccttcttcctcttacggtgcgccgggcggtggcaacggtggccgtccgtctgacacctacggtgcgccgggtggcggtaacggtggccgtccttcttcctcttacggtgcgccgggcggtggcaacggtggccgtccgtctgacacctacggtgcgccgggtggcggtaacggtggccgtccttcttcctcttac

SEQ ID NO: 3 - protein, 575 aa, Drosophila melanogaster
MFKLLGLTLLMAMVVLGRPEPPVNSYLPPSDSYGAPGQSGPGGRPSDSYGAPGGGNGGRPSDSYGAPGQGQGQGQGQGGYAGKPSDTYGAPGGGNGNGGRPSSSYGAPGGGNGGRPSDTYGAPGGGNGGRPSDTYGAPGGGGNGNGGRPSSSYGAPGQGQGNGNGGRSSSSYGAPGGGNGGRPSDTYGAPGGGNGGRPSDTYGAPGGGNNGGRPSSSYGAPGGGNGGRPSDTYGAPGGGNGNGSGGRPSSSYGAPGQGQGGFGGRPSDSYGAPGQNQKPSDSYGAPGSGNGNGGRPSSSYGAPGSGPGGRPSDSYGPPASGSGAGGAGGSGPGGADYDNDIVEYEADQQGYRPQIRYEGDANDGSGPSGPGGPGGQNLGADGYSSGRPGNGNGNGNGGYSGGRPGGQDLGPSGYSGGRPGGQDLGAGGYSNGKPGGQDLGPGGYSGGRPGGQDLGRDGYSGGRPGGQDLGASGYSNGRPGGNGNGGSDGGRVIIGGRVIGGQDGGDQGYSGGRPGGQDLGRDGYSSGRPGGRPGGNGQDSQDGQGYSSGRPGQGGRNGFGPGGQNGDNDGSGYRY

SEQ ID NO: 4 - protein, 7 aa, Artificial Sequence
  Consensus Sequence
  misc_feature (2)..(3): Xaa can be any naturally occurring amino acid
  misc_feature (6)..(6): Xaa can be any naturally occurring amino acid
SXXYGXP

SEQ ID NO: 5 - protein, 15 aa, Artificial Sequence
  Consensus sequence
  misc_feature (1)..(3): Xaa can be any naturally occurring amino acid or no amino acid
  misc_feature (4)..(4): Xaa is proline, alanine, threonine or serine
  misc_feature (6)..(7): Xaa is a charged or polar amino acid
  misc_feature (10)..(10): Xaa is proline, alanine, threonine or serine
  misc_feature (12)..(12): Xaa is glycine, alanine, threonine or serine
  misc_feature (13)..(14): Xaa is glycine, a polar amino acid or no amino acid
  misc_feature (15)..(15): Xaa can be any naturally occurring amino acid or no amino acid
XXXXSXXYGXPXXXX

SEQ ID NO: 6 - protein, 15 aa, Artificial Sequence
  Repeat unit of resilin protein
GAPGGGNGGRPSDTY

SEQ ID NO: 7 - protein, 15 aa, Artificial Sequence
  Repeat unit of resilin protein
GAPGGGNGGRPSSSY

SEQ ID NO: 8 - DNA, 90 bp, Artificial Sequence
  Synthetic oligonucleotide
ggtgcgccgggcggtggcaacggtggccgtccgtctgacacctacggtgcgccgggtggcggtaacggtggccgtccttcttcctcttac

SEQ ID NO: 9 - protein, 11 aa, Artificial Sequence
  Consensus sequence
  misc_feature (1)..(1): Xaa is proline, alanine, threonine or serine
  misc_feature (3)..(4): Xaa is a charged or polar amino acid
  misc_feature (7)..(7): Xaa is proline, alanine, threonine or serine
  misc_feature (9)..(9): Xaa is glycine, alanine, threonine or serine
  misc_feature (10)..(11): Xaa is glycine, a polar amino acid or no amino acid
XSXXYGXPXXX